(19) United States
(12) Patent Application Publication
(10) Pub. No.: US 2023/0272359 A1
(43) Pub. Date: Aug. 31, 2023

(54) METHODS AND COMPOSITIONS FOR TREATMENT AND PREVENTION OF CORONAVIRUS INFECTION

(71) Applicant: Dana-Farber Cancer Institute, Inc., Brookline, MA (US)

(72) Inventors: James Torchia, Boston, MA (US); Gordon J. Freeman, Brookline, MA (US)

(21) Appl. No.: 18/016,209

(22) PCT Filed: Jul. 20, 2021
(86) PCT No.: PCT/US21/42372

§ 371 Date: Jan. 13, 2023

Related U.S. Application Data
(60) Provisional application No. 63/150,686, filed on Feb. 18, 2021.
(60) Provisional application No. 63/053,817, filed on Jul. 20, 2020.

(51) Int. Cl. C12N 9/48; C07K 16/10; C12N 15/85; A61P 31/14

(52) U.S. Cl. CPC C12N 9/485; C07K 16/10; C12N 15/85; A61P 31/14; C12Y 304/17023; C07K 2319/30

(57) ABSTRACT

The present invention relates, in part, to compositions and methods for treating or preventing coronavirus infection.

### CROSS-REFERENCE TO RELATED APPLICATIONS

This application claims the benefit of priority to U.S. Provisional Application Ser. No. 63/053,817, filed on 20 Jul. 2020, and U.S. Provisional Application Ser. No. 63/150,686, filed on 18 Feb. 2021; the entire contents of each application are incorporated herein in their entirety by this reference.

### BACKGROUND OF THE INVENTION

COVID-19, caused by the novel coronavirus SARS-CoV-2, has caused hundreds of thousands to fall ill or die during the global pandemic. Vaccines are being developed but remain at least 18 months away from clinical translation and face the challenges of demonstrating both safety and efficacy in large clinical studies. Depending upon the efficacy and availability of vaccines, infections and deaths may continue for some period thereafter.

Clinical trials of existing antiviral small molecules and disease modifying immune modulators are underway, but their safety, efficacy, and the magnitude of any potential efficacy remain uncertain. Neutralizing antibodies are also being developed and offer the promise of passive immunotherapy that could be delivered at scale. Although a promising approach, the efficacy of neutralizing antibodies relies on their ability to bind a discrete epitope on the SARS-CoV-2 spike protein and prevent the spike protein from binding to its cell surface receptor, angiotensin converting enzyme 2 (ACE2). The reliance of antibody binding to a discrete spike protein epitope means that alterations in spike protein sequence that inhibit antibody binding while preserving ACE2 binding could allow the virus to escape neutralization. Therefore, neutralizing antibodies may not be effective against mutant variants of SARS-CoV-2 that might arise in a large population of infected individuals, especially if widespread treatment with neutralizing antibodies exerts evolutionary pressure on the virus. Similarly, neutralizing antibodies may not be effective against future ACE2-directed novel coronaviruses that have been subject to genetic drift.

Furthermore, pulmonary complications are a major contributor to the morbidity and mortality of COVID-19. Although the etiologies underlying pulmonary complications, including the acute respiratory distress syndrome (ARDS), are complex and multifactorial, an immunopathologic phenomenon known as antibody-dependent enhancement (ADE) has been postulated as a contributing mechanism in COVID-19 patients with ARDS. ADE is a phenomenon seen in some viral syndromes in which neutralizing antibodies may actually worsen infection and inflammation by promoting viral infection or overstimulation of immune cells that express Fcγ receptors (Iwasaki et al. (2020) Nat. Rev. Immunol. 20: 339-42; Taylor et al. (2015) Immunol. Rev. 268: 340-364).

Accordingly, there remains a great need for pharmacological inhibitors of SARS-CoV2 for the treatment and prevention of COVID-19.

### SUMMARY OF THE INVENTION

The present invention is based, at least in part, on the discovery that an inactive, soluble ACE2 protein competitively inhibits the SARS-CoV-2 virus from binding to endogenous ACE2, thereby preventing the virus from entering a cell.

One aspect of the present invention provides an ACE2-Fc fusion polypeptide comprising an ACE2 extracellular domain polypeptide or fragment thereof, a hinge polypeptide, and a fragment crystallizable (Fc) domain or fragment thereof, wherein the ACE2 extracellular domain is enzymatically inactive, and wherein the Fc domain or fragment thereof has attenuated binding affinity for a Fcγ receptor.

Numerous embodiments are further provided that can be applied to any aspect of the present invention and/or combined with any other embodiment described herein. For example, in one embodiment, the ACE2 extracellular domain polypeptide comprises an amino acid sequence having at least 90% identity to the amino acid sequence of any one of SEQ ID NOs: 1-4. In another embodiment, the ACE2 extracellular domain polypeptide comprises at least one amino acid substitution at a residue position selected from the group consisting of H374, H378, R273, H345, H345, H505, H505, R169, W271, and K481. In still another embodiment, the ACE2 extracellular domain polypeptide comprises at least one amino acid substitution selected from the group consisting of H374N, H378N, R273Q, H345A, H345L, H505A, H505L, R169Q, W271Q, and K481Q relative to a wild type ACE2 polypeptide. In yet another embodiment, the at least one amino acid substitution is a H374N or H378N substitution relative to a wild type ACE2 polypeptide. In another embodiment, the at least one amino acid substitution is a H374N and H378N substitution relative to a wild type ACE2 polypeptide. In still another embodiment, the the at least one amino acid substitution is R273Q, H345A, H345L, H505A, H505L amino acid substitutions. In yet another embodiment, the at least one amino acid substitution is R169Q, W271Q, and K481Q amino acid substitutions. In another embodiment, the ACE2 extracellular domain has affinity for a coronavirus. In still another embodiment, the coronavirus is selected from the group consisting of SARS-CoV-1 and the SARS-CoV-2. In another embodiment, the Fc domain comprises an amino acid sequence comprising at least one amino acid substitution relative to a wild-type Fc domain that decreases or eliminates binding of the Fc domain to a Fc receptor. In still another embodiment, the Fc receptor is a FcγIIa receptor. In another embodiment, the Fc domain comprises an amino acid sequence from Table 5. In still embodiment, the Fc domain is derived from an IgG4 antibody. In yet another embodiment, the Fc domain comprises at least one amino acid substitution at a residue position selected from the group consisting of L235, and P329. In another embodiment, the ACE2-Fc fusion polypeptide comprises an S228P or L235E amino acid substitution. In still another embodiment, the ACE2-Fc fusion polypeptide comprises an S228P and L235E amino acid substitution. In yet another embodiment, the Fc domain comprises an amino acid sequence of any one of SEQ ID NOs: 33-36. In another embodiment, the Fc domain is derived from an IgG1 antibody. In still another embodiment, the Fc domain comprises at least one amino acid substitution selected from the group consisting of L234A, L235A, N297A, N297D, and P329G. In yet another embodiment, the Fc domain comprises an amino acid sequence have at least 90% sequence identity to any one of SEQ ID NOs: 37-42 or SEQ ID NO: 55. In another embodiment, the Fc domain is derived from an IgG2 antibody. In still another embodiment, the Fc domain comprises the amino acid sequence of SEQ ID NO: 42. In yet another embodiment, the hinge region comprises an amino acid sequence from Table 2, 3, or 4. In another embodiment, the hinge region consists of a proline or a cysteine-proline dipeptide.

Another aspect of the invention provides an ACE2-Fc fusion polypeptide comprising an amino acid sequence having at least 90% identity to SEQ ID NO: 48, 49, 56, or 57.

Yet another aspect of the invention provides a nucleic acid molecule encoding the ACE2-Fc fusion polypeptide of any of the above aspects.

In another aspect, a nucleic acid molecule is provided that comprises a nucleotide sequence having at least 90% identity to SEQ ID NO: 50, 51, 58, or 59. Numerous embodiments are further provided that can be applied to any aspect of the present invention and/or combined with any other embodiment described herein. For example, one embodiment provides a vector comprising the nucleic acid molecule of either of the immediately preceding aspects. In another embodiment, the vector is an expression vector. In still another embodiment, a cell is provided that comprises the vector of either of the immediately preceding embodiments. Another embodiment provides a cell comprising the vector of either of the immediately preceding embodiments. In still another embodiment, the cell is mammalian cell.

Another aspect of the present invention provides a method of sequestering a coronavirus comprising contacting a fluid comprising a coronavirus with the ACE2-Fc fusion polypeptide of any one of the above aspects, wherein the ACE2-Fc fusion polypeptide binds the coronavirus, thereby sequestering the coronavirus.

Numerous embodiments are further provided that can be applied to any aspect of the present invention and/or combined with any other embodiment described herein. For example, in one embodiment, the sequestered coronavirus is incapable of binding to a full length ACE2 polypeptide. In one embodiment, the fluid is an interstitial fluid, blood, plasma, serum, mucous, cerebrospinal fluid, or lymph. In another embodiment of the methods described herein, the coronavirus is selected from the group consisting of SARS-CoV-1 and SARS-CoV-2.

Still another aspect of the present invention provides a method of inhibiting a coronavirus from binding to an endogenous ACE2 polypeptide expressed by a cell, the method comprising contacting a fluid in communication with the cell with the ACE2-Fc fusion polypeptide of any one of the above aspects, wherein the ACE2-Fc fusion polypeptide binds the coronavirus, thereby inhibiting the coronavirus from binding endogenously expressed ACE2 polypeptides. In one embodiment, the fluid is an interstitial fluid, blood, plasma, serum, mucous, cerebrospinal fluid, or lymph. In another embodiment, the coronavirus is selected from the group consisting of SARS-CoV-1 and SARS-CoV-2. In another embodiment, the cell is a mammalian cell. In still another embodiment, the mammalian cell is a human cell.

Another aspect provides a method for treating a subject having or suspected of having a coronavirus infection, the method comprising administering a therapeutically effective amount of a pharmaceutical composition comprising the ACE2-Fc fusion polypeptide of any one of the above aspects to the subject. In one embodiment, the coronavirus is selected from the group consisting of SARS-CoV-1 and SARS-CoV-2. In another embodiment, the subject is a mammal. In another embodiment, the subject is a human. In still another embodiment, the administering is selected from the group consisting of subcutaneous, intravenous, parenteral, intraperitoneal, intrathecal, oral, inhalation, nebulization, and transdermal.

In still another aspect, a method is provided for preventing a coronavirus infection in a subject at risk of infection, the method comprising administering an effective amount of a pharmaceutical composition comprising the ACE2-Fc fusion polypeptide of any one of the above aspects to the subject. In one embodiment, the coronavirus is selected from the group consisting of SARS-CoV-1 and SARS-CoV-2. In another embodiment, the subject is a mammal. In another embodiment, the subject is a human. In still another embodiment, the administering is selected from the group consisting of subcutaneous, intravenous, parenteral, intraperitoneal, intrathecal, oral, inhalation, nebulization, and transdermal.

Another aspect of the present invention provides a method of treating or preventing antibody dependent enhancement of a coronavirus infection in a subject, the method comprising administering to the subject a therapeutically effective amount of a pharmaceutical composition comprising the ACE2-Fc fusion polypeptide of any one of the above aspects to the subject.

Numerous embodiments are further provided that can be applied to any aspect of the present invention and/or combined with any other embodiment described herein. For example, in one embodiment, the coronavirus infection is selected from the group consisting of SARS-CoV-1 infection and SARS-CoV-2 infection. In one embodiment, the subject is a mammal. In another embodiment, the subject is a human. In still another embodiment, the administering is selected from the group consisting of subcutaneous, intravenous, parenteral, intraperitoneal, intrathecal, oral, inhalation, nebulization, and transdermal.

Numerous additional embodiments are provided that can be applied to any aspect of the present invention and/or combined with any other embodiment described herein. For example, in an embodiment, the coronavirus: is resistant to neutralization by a monoclonal antibody capable of neutralizing other coronaviruses; is a variant of SARS-CoV-2 that is resistant to neutralization by a monoclonal antibody capable of neutralizing SARS-CoV-2; is resistant to the immunity imparted by a coronavirus vaccine; is a variant of SARS-CoV-2 that is resistant to the immunity imparted by a SARS-CoV-2 vaccine; is resistant to natural immunity imparted by prior coronavirus infection; is a variant of SARS-CoV-2 that is resistant to natural immunity imparted by prior SARS-CoV-2 infection; harbors an E484 substitution in the S-protein; harbors a N501 substitution in the S-protein; harbors a K417 substitution in the S-protein; harbors E484 and N501 substitutions in the S-protein; harbors an E484K substitution in the S-protein; harbors an E484Q substitution in the S-protein; harbors a N501Y substitution in the S-protein; harbors a K417N substitution in the S-protein; harbors E484K and N501Y substitutions in the S-protein; harbors E484, N501, and K417 substitutions in the S-protein; harbors E484K, N501Y, and K417N substitutions in the S-protein; harbors an L452 substitution in the S-protein; harbors an L452R substitution in the S-protein; harbors a T478 substitution in the S-protein; harbors a T478K substitution in the S-protein; harbors an L452 and a T478 substitution in the S-protein; harbors an L452R and a T478K substitution in the S-protein; descends from the B.1.1.7 lineage, also known as 20I/501Y.V1, the “British variant,” or Alpha variant; is the B.1.1.7 lineage, also known as 20I/501Y.V1, the “British variant,” or Alpha variant; descends from the B.1.351 lineage, also known as 20H/501Y.V2, the “South African COVID-19 variant,” or Beta variant (harbors E484K, N501Y, and K417N substitutions in addition to other substitutions outside of the S-protein receptor binding domain); is the B.1.351 lineage, also known as 20H/501Y.V2, the “South African COVID-19 variant,” or Beta variant (harbors E484K, N501Y, and K417N substitutions in addition to other substitutions outside of the S-protein receptor binding domain); descends from the B.1.1.248 lineage, also known as the “Brazilian COVID-19 variant,” lineage P.1, or Gamma variant (harbors E484K and N501Y substitutions in addition to other substitutions outside of the S-protein receptor binding domain); is the B.1.1.248 lineage, also known as the “Brazilian COVID-19 variant,” lineage P.1, or Gamma variant (harbors E484K and N501Y substitutions in addition to other substitutions outside of the S-protein receptor binding domain); descends from the B.1.617 lineage; descends from the B.1.617.1 lineage (harbors an E484Q substitution); is the B.1.617.1 lineage (harbors an E484Q substitution); descends from the B.1.617.2 lineage, also known as Delta variant (harbors L452R and T478K substitutions in addition to other S-protein mutations); is the B.1.617.2 lineage, also known as Delta variant (harbors L452R and T478K substitutions in addition to other S-protein mutations); descends from the B.1.617.3 lineage (harbors E484Q substitution); and/or is the B.1.617.3 lineage (harbors E484Q substitution).

### BRIEF DESCRIPTION OF THE DRAWINGS

FIG. 1 is a three-dimensional illustration of an ACE2-Fc fusion polypeptide.

FIG. 2A and FIG. 2B characterize the ability of two ACE2-Fc fusion polypeptides to bind to SARS-CoV-2. FIG. 2A is a binding curve that shows DF-COV-01 and DF-COV-02 avidly bind SARS-CoV-2. The concentrations of DF-COV-01 and DF-COV-02 are measured in μg/ml. FIG. 2B is the same binding curve shown in FIG. 2A but the concentrations of DF-COV-01 and DF-COV-02 are measured in molarity.

FIG. 3 comprises a graph that characterize the ability of two ACE2-Fc fusion polypeptides to neutralize pseudotyped SARS-CoV-2 viral particles. DF-COV-01 and DF-COV-02 inhibit viral entry into ACE2-expressing 293T cells.

FIG. 4A and FIG. 4B compare the affinity of two ACE2-Fc fusion polypeptides for immobilized recombinant human FcγRIIa with the affinity of purified polyclonal human IgG for immobilized recombinant human FcγRIIa. FIG. 4A is a binding curve that shows DF-COV-01 and DF-COV-02 have markedly attenuated FcγRIIa binding when compared with human IgG. The concentrations of DF-COV-01 and DF-COV-02 are measured in μg/ml. FIG. 4B is the same binding curve shown in FIG. 4A but the concentrations of DF-COV-01 and DF-COV-02 are measured in molarity.

FIG. 5 compares ACE2 enzymatic activity of DF-COV-01 and DF-COV-02 with that of two ACE2-Fc fusions containing wild-type ACE2 extracellular domains. DF-COV-01 and DF-COV-02 exhibit no enzymatic activity, whereas the matched wild-type ACE2-Fc fusions do exhibit enzymatic activity.

FIG. 6A and FIG. 6B characterize the ability of DF-COV-01 and DF-COV-02 to neutralize pseudotyped SARS-CoV-2 viral particles containing both luciferase and GFP reporter genes. ACE2-expressing 293T cells were used as targets. In FIG. 6A, infection was measured by a luminescence assay detecting luciferase expression. In FIG. 6B, infection of 293T cells was visualized by fluorescence microscopy detecting GFP expression. Both DF-COV-01 and DF-COV-02 inhibit viral entry into ACE2-expressing 293T cells, with DF-COV-01 neutralizing more potently than DF-COV-02.

FIG. 7 characterizes the ability of DF-COV-01 and DF-COV-02 to neutralize wild-type SARS-CoV-2 virus and prevent infection of human alveolar type 2 cells (iAT2) derived from human induced pluripotent stem cells. Infection of iAT2 cells by SARS-CoV-2 was detected by a fluorescently labeled antibody against the SARS-CoV-02 N-protein and measured by flow cytometry. Both DF-COV-01 and DF-COV-02 inhibited infection, but a negative control Fc fusion (PD-L1-Fc) did not.

FIG. 8 is a graph showing the pharmacokinetic (PK) curves of DF-COV-01 and DF-COV-02 in hamsters when administered via intraperitoneal injection. The serum half-life (t_{1/2}) of DF-COV-01 is longer than that of DF-COV-02. The volume of distribution (Va.) of DF-COV-02 is larger than that of DF-COV-01. DF-COV-02 is believed to penetrate peripheral tissue to a greater degree than DF-COV-01.

FIG. 9A-FIG. 9E characterize the activity of DF-COV-01, DF-COV-02, DF-COV-03, and DF-COV-04 against the SARS-CoV-2 virus in vivo. Syrian hamsters were challenged intranasally with SARS-CoV-2 and treated for a total of 3 days with either 40 mg/kg of DF-COV-01 once daily or 20 mg/kg of DF-COV-02, DF-COV-03, DF-COV-04, or vehicle control (PBS) twice daily. FIG. 9A compares the oropharyngeal viral titers at day 3, FIG. 9B compares the nasal turbinate viral titers at day 3, FIG. 9C compares the lung titers at day 3, FIG. 9D compares the body weights of hamsters in each group at day 3 (relative to each individual's body weight at day 0), and FIG. 9E compares the body weights over time of hamsters treated with DF-COV-01 with those treated with vehicle control (PBS). DF-COV-01 reduced oropharyngeal titer, nasal turbinate titer, and lung titer compared with vehicle control (PBS). Weight loss is a manifestation of SARS-CoV-2 infection in hamsters. Hamsters treated with DF-COV-01 had a higher mean body weight at day 3 compared with hamsters treated with vehicle control (PBS). Several hamsters treated with DF-COV-01 began to recover lost body weight by day 3, when all hamsters were sacrificed.

FIG. 10A and FIG. 10B comprise schematic and three-dimensional illustrations of DF-COV-01 and DF-COV-02. FIG. 10A depicts DF-COV-01, an ACE2-Fc fusion with an enzymatically inactive full-length ACE2 extracellular domain, ACE2-NN(18-740), fused to a silent IgG4-SPLE Fc domain. FIG. 10B depicts DF-COV-02, an ACE2-Fc fusion with an enzymatically inactive ACE2 metallopeptidase domain, ACE2-NN(18-612), fused to a silent IgG4-SPLE Fc domain. It can be appreciated that the membrane-proximal stalk of the ACE2 extracellular domain may enhance the flexibility of DF-COV-01. It also can be appreciated that DF-COV-02 is smaller and its peripheral tissue penetration may be enhanced by this smaller molecular weight and smaller hydrodynamic radius.

FIG. 11 comprises a schematic illustration of DF-COV-03, an ACE2-Fc fusion with an enzymatically inactive ACE2 metalloprotease domain, ACE2-NN(18-615), fused to a silent IgG1-LALA Fc domain. The hinge region of the IgG1 Fc domain is more flexible than that of the IgG4 Fc domain and a flexible G4AG4 artificial linker sequence is also included.

FIG. 12A and FIG. 12B comprise a three-dimensional illustration comparing the distances between the aminotermini and carboxytermini of two ACE2 metalloprotease domains binding to a SARS-CoV-2 S-protein, as well as a schematic illustration of an ACE2-Fc fusion designed based on this comparison. In FIG. 12A, the RBDs of two crystal structures of the ACE2 metalloprotease domain binding to the SARS-CoV-2 S-protein RBD (PDB: 6M0J) were overlayed with the two RBDs in the “up” conformation in the full-length SARS-CoV-2 S-protein structure (PDB: 6X2B). The distance between the aminotermini of the ACE2 metalloprotease domains was measured to be 40A and the distance between the carboxytermini of the ACE2 metalloprotease domains was measured to be 93A. FIG. 12B is a schematic illustration of DF-COV-04, an ACE2-Fc fusion with an enzymatically inactive ACE2 metalloprotease domain, ACE2-NN(18-615), fused to a silent IgG1-LALA Fc domain. The orientation of the ACE2 and Fc domains of this polypeptide are reversed, with the Fc domain being aminoterminal to the ACE2 domain. When two ACE2 metalloprotease domains of DF-COV-04 simultaneously bind two RBDs on the same viral S-protein, the linker may be required to span a shorter distance, which is more energetically favorable. Thus, ACE2-Fc fusions with the ACE2 metalloprotease domain located carboxyterminal to the Fc domain may improve the potency of viral neutralization.

FIG. 13A and FIG. 13B characterize the ability of DF-COV-01, DF-COV-02, DF-COV-03, and DF-COV-04 to neutralize pseudotyped SARS-CoV-2 viral particles. ACE2-expressing 293T cells were used as targets and infection is measured by a luminescence assay detecting luciferase reporter expression. FIG. 13A comprises neutralization curves with concentrations in μg/mL. FIG. 13B comprises neutralization curves with concentrations in nanomolar units. DF-COV-01 most potently neutralized virus (IC50 1.8 nM) and DF-COV-04 also potently neutralized virus (IC50 4.4 nM). DF-COV-02 and DF-COV-03 neutralized virus less potently (IC50 10 nM and 15 nM, respectively).

FIG. 14A and FIG. 14B characterize the ability of DF-COV-01, DF-COV-02, DF-COV-03, and DF-COV-04 to bind to the SARS-CoV-2 S-protein. FIG. 14A comprises binding curves with concentrations in μg/mL and FIG. 14B comprises binding curves with concentrations in nanomolar units. DF-COV-01 and DF-COV-02 have equivalent binding to the S-protein. DF-COV-03 has a slightly higher avidity for the S-protein than DF-COV-01 and DF-COV-02. Surprisingly, DF-COV-04 has a lower avidity for the S-protein as measured by this assay even though it demonstrated more potent vial neutralization than DF-COV-02 and DF-COV-03.

FIG. 15A and FIG. 15B measure binding affinity of DF-COV-01, DF-COV-02, DF-COV-03, and DF-COV-04 to the SARS-CoV-2 S-protein. Affinity was determined by bio-layer interferometry (Octet) with DF-COV compounds immobilized and SARS-CoV-2 S-protein in solution. DF-COV-01 and DF-COV-02 had a higher affinity for the SARS-CoV-2 S-protein than DF-COV-03 and DF-COV-04.

FIG. 16A-FIG. 16D characterize the stability of DF-COV-01, DF-COV-02, DF-COV-03, and DF-COV-04 after nebulization. Each compound was nebulized with a vibrating mesh nebulizer and then collected in a microcentrifuge tube. The binding of nebulized compound to immobilized SARS-CoV-2 S-protein was compared with that of a sample taken prior to nebulization (baseline). Binding was detected via an anti-human IgG-HRP secondary antibody. For all four DF-COV compounds there was no significant difference in S-protein binding avidity between nebulized sample and non-nebulized sample (baseline).

FIG. 17 compares the serum half-life of DF-COV-01, DF-COV-02, DF-COV-03, and DF-COV-04 in Syrian hamsters. 8 mg/kg of each of the DF-COV compounds was administered via intraperitoneal injection to separate groups of three hamsters at time zero. Venous blood draws were performed at 4, 8, 24, 48, and 72 hours and the serum concentration of the DF-COV compounds was determined by ELISA for each sample. DF-COV-01 had the longest serum half-life, which was 52 hours. The other three ACE2-Fc design permutations had much shorter half-lives of approximately 16, 13, and 7 hours.

FIG. 18 compares daily weight trends among Syrian hamsters challenged intranasally with SARS-CoV-2 and treated with either DF-COV-01, DF-COV-02, DF-COV-03, DF-COV-04, or vehicle control (PBS).

FIG. 19A and FIG. 19B demonstrate the ability of DF-COV-01 to treat hamsters in a therapeutic model of SARS-CoV-2 infection, in which hamsters are treated twelve hours after being challenged with 1×10⁴ PFU of SARS-CoV-2. FIG. 19A provides a schematic diagram of the treatment and FIG. 19B provides results.

FIG. 20A and FIG. 20B demonstrate binding characteristics of DF-COV compounds to SARS-CoV-2 variants. FIG. 20A provides numerical data and FIG. 20B provides titration curves.

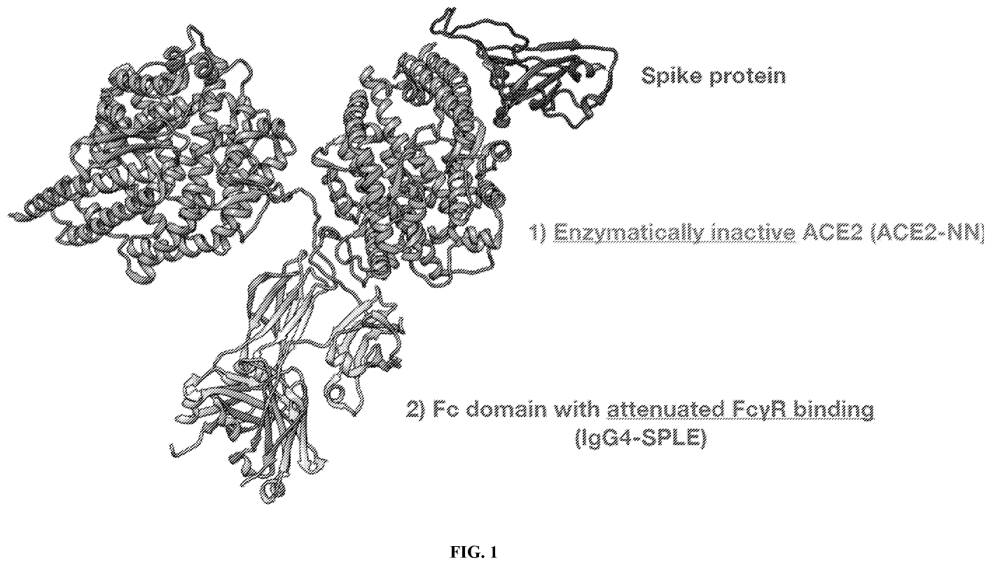

### DETAILED DESCRIPTION OF THE INVENTION

The present invention is based, at least in part, on the discovery that an inactive, soluble ACE2 protein competitively inhibits the SARS-CoV-2 virus from binding to endogenous ACE2, thereby preventing the virus from entering a cell. ACE2-Fc fusion polypeptides encompassed by the present invention potently inhibit SARS-CoV and SARS-CoV-2 and have favorable pharmacokinetics.

Fusion of a surface receptor extracellular domain to an Fc domain is a common method of extending the half-life of surface receptors and allowing them to be used as drugs. Examples of receptor-Fc fusions include etanercept, abatacept, belatacept, alefacept, and rilonacept, among others. The half-lives of these drugs range between 3 and 15 days. Fusion to an Fc domain has the added benefit of increasing avidity of the receptor to its binding partner because Fc domains form a dimer, thus two receptor domains will be present on each molecule of the drug, increasing its binding avidity. Fc fusions transit across the vasculature readily because they are chaperoned across endothelial cells by the Fc neonatal receptor, and therefore generally exhibit higher peripheral tissue concentrations.

ACE2 is a single-pass membrane protein, and its extracellular domain can be fused to the Fc domain of IgG to create an Fc-fusion. The concept of utilizing an ACE2-Fc fusion protein to inhibit infection of cells by coronaviruses has previously been demonstrated. In 2004, Moore et al. at Harvard Medical School demonstrated that an ACE2-Fc fusion could potently inhibit the infection of target cells by SARS-CoV pseudovirus in vitro with an IC50 of approximately 2 nM ((2004) J. Virol., 78(19):10628-35). More recently, in February 2020, Lei et al. at the Second Military Medical University in Shanghai, China confirmed these findings and demonstrated that an ACE2-Fc fusion inhibits SARS-CoV-2 even more potently, with an IC50 of less than 0.1 μg/mL, or less than 0.5 nM ((2020) Nat. Commun. 11(1):2070). These preclinical studies demonstrate that soluble ACE2-Fc can successfully compete with cell-surface ACE2 to prevent infection of target cells. This IC50 in the low to sub-nanomolar range implies a highly avid interaction between ACE2 and the spike protein of SARS-CoV-2. The IC50 of ACE2-Fc is similar to that of a neutralizing monoclonal antibody. For instance, Wang et al. recently reported SARS-CoV-2 neutralizing antibody with a similar IC50 of approximately 0.5 nM (Wang et al. (2020) Nat. Commun. 11:225). The fusion proteins encompassed by the present invention go further by using an enzymatically dead ACE2 extracellular domain fused to an Fc domain fragment with reduced affinity for Fey receptors.

As described further herein, the ACE2-Fc fusion proteins encompassed by the present invention have a number of advantages, including 1) allowance of ambulatory treatment and prophylaxis because the pharmacokinetics allow for efficacious administration (e.g., subcutaneous dosing), 2) avoidance of hemodynamic side effects because inactive ACE2 avoids hypotension from RAAS pathway inhibition, 3) mitigation of antibody-dependent enhancement (ADE) risk because a silent Fc domain reduces the risk of innate immune cell infection and interuption of nautrally occurring ADE, and 4) efficacious targeting of ACE2-directed novel coronaviruses because defined epitopes are not relied upon, unlike neutralizing antibodies and vaccines.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, nebulization, and transdermal routes. The injection can be bolus injections or can be continuous infusion. Depending on the route of administration, the agent can be coated with or disposed in a selected material to protect it from natural conditions that may detrimentally affect its ability to perform its intended function. The agent may be administered alone, or in conjunction with a pharmaceutically acceptable carrier. The agent also may be administered as a prodrug, which is converted to its active form in vivo.

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., an ACE2-Fc fusion polypeptide encompassed by the present invention). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, protein subunit peptide and a C-terminal polyhistidine tag to make bivalent and biotinylated scFv polypeptides (Kipriyanov et al. (1994) Mol. Immunol. 31:1047-1058). Antibody portions, such as Fc fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

As used herein, the term “antibody dependent enhancement (ADE)” refers to the worsening of a disease or condition (i.e., a coronavirus infection) caused by antibody-assisted virus entry into a cell. For example, the Fc domain of a neutralizing antibody that are specifically bound to a SARS-CoV-2 antigen may be recognized by an Fcγ receptor (e.g., a FcγIIa receptor). Upon binding of the Fcγ receptor to the Fc domain, the receptor mediates entry of the antibody and the bound virus into the cell. Instead of the virus then being destroyed within the cell, the viral entry results in increased cellular infection and inflammation. Typically, ADE is mediated by FcγIIa receptors.

As used herein, the term “isotype” refers to the antibody class (e.g., IgM, IgG1, IgG2C, and the like) that is encoded by heavy chain constant region genes.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

As used herein, the term “inhibiting” and grammatical equivalents thereof refer to decreasing, limiting, and/or blocking a particular action, function, or interaction. A reduced level of a given output or parameter need not, although it may, mean an absolute absence of the output or parameter. The invention does not require, and is not limited to, methods that wholly eliminate the output or parameter. The given output or parameter can be determined using methods well-known in the art, including, without limitation, immunohistochemical, molecular biological, cell biological, clinical, and biochemical assays, as discussed herein and in the examples. The opposite terms “promoting,” “increasing,” and grammatical equivalents thereof refer to the increase in the level of a given output or parameter that is the reverse of that described for inhibition or decrease.

As used herein, the term “interacting” or “interaction” means that two molecules (e.g., protein, nucleic acid), or fragments thereof, exhibit sufficient physical affinity to each other so as to bring the two interacting molecules, or fragments thereof, physically close to each other. An extreme case of interaction is the formation of a chemical bond that results in continual and stable proximity of the two entities. Interactions that are based solely on physical affinities, although usually more dynamic than chemically bonded interactions, can be equally effective in co-localizing two molecules. Examples of physical affinities and chemical bonds include but are not limited to, forces caused by electrical charge differences, hydrophobicity, hydrogen bonds, Van der Waals force, ionic force, covalent linkages, and combinations thereof. The state of proximity between the interaction domains, fragments, proteins or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, proteins, or entities. Examples of interactions include specific interactions between antigen and antibody, ligand and receptor, enzyme and substrate, and the like.

Generally, such an interaction results in an activity (which produces a biological effect) of one or both of said molecules. The activity may be a direct activity of one or both of the molecules, (e.g., signal transduction). Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

An “interaction” between two molecules, or fragments thereof, can be determined by a number of methods. For example, an interaction can be determined by functional assays. Such as the two-hybrid Systems. Protein-protein interactions can also be determined by various biophysical and biochemical approaches based on the affinity binding between the two interacting partners. Such biochemical methods generally known in the art include, but are not limited to, protein affinity chromatography, affinity blotting, immunoprecipitation, and the like. The binding constant for two interacting proteins, which reflects the strength or quality of the interaction, can also be determined using methods known in the art. See Phizicky and Fields, (1995) Microbiol. Rev., 59:94-123.

As used herein, a “kit” is any manufacture (e.g. a package or container) comprising at least one reagent, e.g. a probe, for specifically detecting or modulating the expression of a human or humanized ACE2-Fc fusion polypeptide encompassed by the present invention. The kit may be promoted, distributed, or sold as a unit for performing the methods encompassed by the present invention.

As used herein, the term “modulate” includes up-regulation and down-regulation, e.g., enhancing or inhibiting the expression and/or activity of the ACE2-Fc fusion polypeptide encompassed by the present invention.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a polypeptide or fragment thereof, in which the protein is separated from cellular components of the cells from which it is isolated or recombinantly produced. In one embodiment, the language “substantially free of cellular material” includes preparations of an ACE2-Fc fusion polypeptide or fragment thereof, having less than about 30% (by dry weight) of non-ACE2-Fc fusion protein (also referred to herein as a “contaminating protein”), less than about 20%, less than about 10%, or less than 5%. When a fusion protein or fragment thereof, e.g., a biologically active fragment thereof, is recombinantly produced, it is also substantially free of culture medium, i.e., culture medium represents less than about 20%, less than about 10%, or less than about 5% of the volume of the protein preparation.

As used herein, the term “nucleic acid molecule” is intended to include DNA molecules and RNA molecules. A nucleic acid molecule may be single-stranded or double-stranded DNA. As used herein, the term “isolated nucleic acid molecule” is intended to refer to a nucleic acid molecule in which the nucleotide sequences are free of other nucleotide sequences, which other sequences may naturally flank the nucleic acid in human genomic DNA.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 81%, 82%, 83%, 84%, 85%, 86%, 87%, 88%, 89%, 90%, 91%, 92%, 93%, 94%, 95%, 96%, 97%, 98%, 99% or more of the nucleotides. Alternatively, substantial homology exists when the segments will hybridize under selective hybridization conditions, to the complement of the strand.

The percent identity between two sequences is a function of the number of identical positions shared by the sequences (i.e., % identity=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences encompassed by the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules encompassed by the present invention. BLAST protein searches can be performed with the XBLAST program, score=50, wordlength=3 to obtain amino acid sequences homologous to the protein molecules encompassed by the present invention. To obtain gapped alignments for comparison purposes, Gapped BLAST can be utilized as described in Altschul et al., (1997) Nucleic Acids Res. 25(17):3389 3402. When utilizing BLAST and Gapped BLAST programs, the default parameters of the respective programs (e.g., XBLAST and NBLAST) can be used (available on the world wide web at the NCBI website).

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well-known in the art (see, F. Ausubel, et al., ed. Current Protocols in Molecular Biology, Greene Publishing and Wiley Interscience, New York (1987)).

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of an ACE2-Fc fusion nucleic acid and normal post-transcriptional processing (e.g. splicing), if any, of the RNA transcript, and reverse transcription of the RNA transcript.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

If not stated otherwise, the term “compound” as used herein includes but is not limited to peptides, nucleic acids, carbohydrates, natural product extract libraries, organic molecules, such as small organic molecules, inorganic molecules, including but not limited to chemicals, metals and organometallic molecules.

The terms “derivatives,” “analogs,” or “variants” as used herein include, but are not limited, to polypeptides comprising regions that are substantially homologous to the ACE2 polypeptide, in various embodiments, by at least 30%, 40%, 50%, 60%, 70%, 80%, 90%, 95% or 99% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or nonstringent conditions. It means a protein which is the outcome of a modification of the naturally occurring protein, by amino acid substitutions, deletions and additions, respectively, which derivatives still exhibit the biological function of the naturally occurring protein although not necessarily to the same degree. The biological function of such proteins can e.g. be examined by suitable available in vitro assays as provided in the invention.

The term “functionally active” as used herein refers to a polypeptide, namely a fragment or derivative, having structural, regulatory, or biochemical functions of the protein according to the embodiment of which this polypeptide, namely fragment or derivative is related to.

“Function-conservative variants” are those in which a given amino acid residue in a protein or enzyme has been changed without altering the overall conformation and function of the polypeptide, including, but not limited to, replacement of an amino acid with one having similar properties (e.g., polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, or at least 75%, at least 85%, at least 90%, or even at least 95%, and which has the same or substantially similar properties or functions as the native or parent protein to which it is compared.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus, internally, or at the carboxyl-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. They can be, for example, at least and/or including 10, 15, 20, 25, 30, 35, 40, 45, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340 or more long so long as they are less than the length of the full-length polypeptide. Alternatively, they can be no longer than and/or excluding such a range so long as they are less than the length of the full-length polypeptide.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, and in some embodiments at least about 75%, at least about 90%, or at least about 95% of the nucleotide residue positions of each of the portions are occupied by the same nucleotide residue. In some embodiments, all nucleotide residue positions of each of the portions are occupied by the same nucleotide residue.

As used herein, the term “host cell” is intended to refer to a cell into which a nucleic acid encompassed by the present invention, such as a recombinant expression vector encompassed by the present invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30%, less than about 20%, less than about 10%, or less than about 5% (by dry weight) of chemical precursors or non-antibody, polypeptide, peptide or fusion protein chemicals.

The term “activity” when used in connection with proteins means any physiological or biochemical activities displayed by or associated with a particular protein including but not limited to activities exhibited in biological processes and cellular functions, ability to interact with or bind another molecule or a moiety thereof, binding affinity or specificity to certain molecules, in vitro or in vivo stability (e.g., protein degradation rate), antigenicity and immunogenecity, enzymatic activities, etc. Such activities may be detected or assayed by any of a variety of suitable methods as will be apparent to skilled artisans.

The terms “polypeptides” and “proteins” are, where applicable, used interchangeably herein. They may be chemically modified, e.g. post-translationally modified. For example, they may be glycosylated or comprise modified amino acid residues. They may also be modified by the addition of a signal sequence to promote their secretion from a cell where the polypeptide does not naturally contain such a sequence. They may be tagged with a tag. Polypeptides/proteins for use in the invention may be in a substantially isolated form. It will be understood that the polypeptide/protein may be mixed with carriers or diluents which will not interfere with the intended purpose of the polypeptide and still be regarded as substantially isolated. A polypeptide/protein for use in the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention.

The terms “hybrid protein”, “hybrid polypeptide,” “hybrid peptide”, “fusion protein”, “fusion polypeptide”, and “fusion peptide” are used herein interchangeably to mean a non-naturally occurring protein having a specified polypeptide molecule covalently linked to one or more polypeptide molecules that do not naturally link to the specified polypeptide. Thus, a “hybrid protein” may be two naturally occurring proteins or fragments thereof linked together by a covalent linkage. A “hybrid protein” may also be a protein formed by covalently linking two artificial polypeptides together. Typically, but not necessarily, the two or more polypeptide molecules are linked or fused together by a peptide bond forming a single non-branched polypeptide chain.

The term “tag” as used herein is meant to be understood in its broadest sense and to include, but is not limited to any suitable enzymatic, fluorescent, or radioactive labels and suitable epitopes, including but not limited to HA-tag, Myc-tag, T7, His-tag, FLAG-tag, Calmodulin binding proteins, glutathione-S-transferase, strep-tag, KT3-epitope, EEF-epitopes, green-fluorescent protein and variants thereof.

The term “ACE2,” also known as “Angiotensin I Converting Enzyme 2” refers to dipeptidyl caroxydipetidase that has significant homology to Angiotensin I Converting Enzyme. The protein converts angiotensin I to angiotensin 1-9 and angiotensin II to angiotensin 1-7 (Donoghue et al. (2000) Circ Res., 87(5):E1-9., Tipnis et al. (2000) J Biol Chem., 275(43):33238-43, Vickers et al. (2002) J Biol Chem., 277(17):14838-43). The ACE2 protein also hydrolyzes apelin-13 and dynorphin-13 with high efficiency (Vickers et al. (2002)). ACE2 efficiently binds the spike (S) protein of coronaviruses, included the virus (SARS CoV-1) that causes severe acute respiratory syndrome (SARS) (Li et al. (2003) Nature 426: 450-454 and the SARS CoV-2, which causes COVID-19 (Hoffman et al. (2020) Cell, Apr. 16;181(2):271-280.e8.

The term “full length ACE2 polypeptide” or “wild-type ACE2 polypeptide” refers to a polypeptide having at least 85% sequence identity to:

| | |
|---|---|
| 1 | MSSSSWLLLS LVAVTAAQST IEEQAKTFLD |
| | KFNHEAEDLF YQSSLASWNY NTNITEENVQ |
| 61 | NMNNAGDKWS AFLKEQSTLA QMYPLQEIQN |
| | LTVKLQLQAL QQNGSSVLSE DKSKRLNTIL |
| 121 | NTMSTIYSTG KVCNPDNPQE CLLLEPGLNE |
| | IMANSLDYNE RLWAWESWRS EVGKQLRPLY |
| 181 | EEYVVLKNEM ARANHYEDYG DYWRGDYEVN |
| | GVDGYDYSRG QLIEDVEHTF EEIKPLYEHL |
| 241 | HAYVRAKLMN AYPSYISPIG CLPAHLLGDM |
| | WGRFWTNLYS LTVPFGQKPN IDVTDAMVDQ |
| 301 | AWDAQRIFKE AEKFFVSVGL PNMTQGFWEN |
| | SMLTDPGNVQ KAVCHPTAWD LGKGDFRILM |
| 361 | CTKVTMDDFL TAHHEMGHIQ YDMAYAAQPF |
| | LLRNGANEGF HEAVGEIMSL SAATPKHLKS |
| 421 | IGLLSPDFQE DNETEINFLL KOALTIVGTL |
| | PFTYMLEKWR WMVFKGEIPK DQWMKKWWEM |
| 481 | KREIVGVVEP VPHDETYCDP ASLFHVSNDY |
| | SFIRYYTRTL YQFQFQEALC QAAKHEGPLH |
| 541 | KCDISNSTEA GQKLFNMLRL GKSEPWTLAL |
| | ENVVGAKNMN VRPLLNYFEP LFTWLKDQNK |
| 601 | NSFVGWSTDW SPYADQSIKV RISLKSALGD |
| | KAYEWNDNEM YLFRSSVAYA MRQYFLKVKN |
| 661 | QMILFGEEDV RVANLKPRIS FNFFVTAPKN |
| | VSDIIPRTEV EKAIRMSRSR INDAFRLNDN |
| 721 | SLEFLGIQPT LGPPNQPPVS IWLIVFGVVM |
| | GVIVVGIVIL IFTGIRDRKK KNKARSGENP |
| 781 | YASIDISKGE NNPGFQNTDD VQTSF |

The term “ACE2” is intended to include fragments, variants (e.g., allelic variants) and derivatives thereof. Representative human ACE2 cDNA and human ACE2 protein sequences are well-known in the art and are publicly available from the National Center for Biotechnology Information (NCBI). Human ACE2 isoforms include the protein NP 001358344.1 encoded by the transcript NM 001371415.1, the protein NP 068576.1 encoded by the transcript NM 021804.3, and the protein AAQ89076.1 encoded by the transcript AY358714.1. Nucleic acid and polypeptide sequences of ACE2 orthologs in organisms other than humans are well-known and include, for example, chimpanzee (XM_016942979.1-XP_016798468.1; and XM_016942980.1-XP_016798469.1), Rhesus monkey (NM 001135696.1-NP 001129168.1; XM_028841825.1-XP_028697658.1; and XM_015126958.2-XP_014982444.2), dog (NM 001165260.1-NP 001158732.1; XM_014111329.2-XP_013966804.1; XM_022415506.1-XP_022271214.1; and XM_005640992.2-XP_005641049.1), cow (NM 001024502.4-NP 001019673.2; XM_005228428.4-XP_005228485.1; XM_015461785.2-XP_015317271.1; XM_005228429.4-XP_005228486.1; and XM_024987850.1-XP_024843618.1), mouse (NM 001130513.1-NP 001123985.1; and NM 027286.4-NP 081562.2), rat (NM 001012006.1-NP 001012006.1), chicken (XM_416822.5-XP_416822.2), frog (XM_002938247.4-XP_002938293.2), zebrafish (NM 001007297.1-NP 001007298.1; XM_005169359.4-XP_005169416.1; and XM_005169360.4-XP_005169417.13.2), and C. elegans (NM 001029282.6-NP 001024453.1).

The term “fragment crystallizable domain” or “Fc domain” refers to the fragment crystallizable region of an IgG antibody. This domain binds to the Fcγ receptor (e.g., a FcγIIa receptor, which allows cellular internalization of the antibody.

As used herein, the term “ACE-2 directed coronavirus” refers to a subset of coronaviruses that use the ACE2 protein to enter cells. At least seven coronaviruses are known to utilize ACE2, including three viruses of global importance: NL63, SARS-CoV, the virus responsible for the 2004 Severe Acute Respiratory Syndrome outbreak, and SARS-CoV-2, the virus responsible for the COVID-19 pandemic (Li et al. (2003) Nature, 426(6965):450-54; Hoffman et al. (2020) Cell, 181(2):271-80 e8.

The term “diagnosing a coronavirus infection” includes the use of the methods, systems, and code of the present invention to determine the presence or absence of a cornovirus or subtypes thereof, a coronavirus polypeptide or nucleic acid molecule encoding said coronavirus polypeptide, or antibodies that specifically bind a coronavirus antigen in an individual. The term also includes methods, systems, and code for assessing the level of disease activity in an individual.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding an ACE2-Fc fusion polypeptide nucleic acid (or any portion thereof) can be used to derive the ACE2-Fc fusion polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the ACE2-fusion polypeptides, and fragments thereof, encompassed by the present invention are known in the art and readily available on publicly available databases, such as the National Center for Biotechnology Information (NCBI). Exemplary nucleic acid and amino acid sequences are provided in Tables 1-8.

TABLE 1
SEQ ID NO: 1 Aamino acid sequences of
ACE2 extracellular domain with H374N
H378N substitutions (residues 18-740)
QSTIEEQAKTFLDKFNHEAEDLFYQSSLASWNYNT
NITEENVQNMNNAGDKWSAFLKEQSTLAQMYPLQE
IQNLTVKLQLQALQQNGSSVLSEDKSKRLNTILNT
MSTIYSTGKVCNPDNPQECLLLEPGLNEIMANSLD
YNERLWAWESWRSEVGKQLRPLYEEYVVLKNEMAR
ANHYEDYGDYWRGDYEVNGVDGYDYSRGQLIEDVE
HTFEEIKPLYEHLHAYVRAKLMNAYPSYISPIGCL
PAHLLGDMWGRFWTNLYSLTVPFGQKPNIDVTDAM
VDQAWDAQRIFKEAEKFFVSVGLPNMTQGFWENSM
LTDPGNVQKAVCHPTAWDLGKGDFRILMCTKVTMD
DFLTAHNEMGNIQYDMAYAAQPFLLRNGANEGFHE
AVGEIMSLSAATPKHLKSIGLLSPDFQEDNETEIN
FLLKQALTIVGTLPFTYMLEKWRWMVFKGEIPKDQ
WMKKWWEMKREIVGVVEPVPHDETYCDPASLFHVS
NDYSFIRYYTRTLYQFQFQEALCQAAKHEGPLHKC
DISNSTEAGQKLFNMLRLGKSEPWTLALENVVGAK
NMNVRPLLNYFEPLFTWLKDQNKNSFVGWSTDWSP
YADQSIKVRISLKSALGDKAYEWNDNEMYLFRSSV
AYAMRQYFLKVKNQMILFGEEDVRVANLKPRISFN
FFVTAPKNVSDIIPRTEVEKAIRMSRSRINDAFRL
NDNSLEFLGIQPTLGPPNQPPVS
SEQ ID NO: 2 Subset of ACE2
extracellular domain with H374N H378N
substitutions (residues 18-708)
QSTIEEQAKTFLDKFNHEAEDLFYQSSLASWNYNT
NITEENVQNMNNAGDKWSAFLKEQSTLAQMYPLQE
IQNLTVKLQLQALQQNGSSVLSEDKSKRLNTILNT
MSTIYSTGKVCNPDNPQECLLLEPGLNEIMANSLD
YNERLWAWESWRSEVGKQLRPLYEEYVVLKNEMAR
ANHYEDYGDYWRGDYEVNGVDGYDYSRGQLIEDVE
HTFEEIKPLYEHLHAYVRAKLMNAYPSYISPIGCL
PAHLLGDMWGRFWTNLYSLTVPFGQKPNIDVTDAM
VDQAWDAQRIFKEAEKFFVSVGLPNMTQGFWENSM
LTDPGNVQKAVCHPTAWDLGKGDFRILMCTKVTMD
DFLTAHNEMGNIQYDMAYAAQPFLLRNGANEGFHE
AVGEIMSLSAATPKHLKSIGLLSPDFQEDNETEIN
FLLKQALTIVGTLPFTYMLEKWRWMVFKGEIPKDQ
WMKKWWEMKREIVGVVEPVPHDETYCDPASLFHVS
NDYSFIRYYTRTLYQFQFQEALCQAAKHEGPLHKC
DISNSTEAGQKLFNMLRLGKSEPWTLALENVVGAK
NMNVRPLLNYFEPLFTWLKDQNKNSFVGWSTDWSP
YADQSIKVRISLKSALGDKAYEWNDNEMYLFRSSV
AYAMRQYFLKVKNQMILFGEEDVRVANLKPRISFN
FFVTAPKNVSDIIPRTEVEKAIRMSR
SEQ ID NO: 3 Subset ACE2 extracellular
domain with H374N H378N substitutions
(residues 18-615)
QSTIEEQAKTFLDKFNHEAEDLFYQSSLASWNYNT
NITEENVQNMNNAGDKWSAFLKEQSTLAQMYPLQE
IQNLTVKLQLQALQQNGSSVLSEDKSKRLNTILNT
MSTIYSTGKVCNPDNPQECLLLEPGLNEIMANSLD
YNERLWAWESWRSEVGKQLRPLYEEYVVLKNEMAR
ANHYEDYGDYWRGDYEVNGVDGYDYSRGQLIEDVE
HTFEEIKPLYEHLHAYVRAKLMNAYPSYISPIGCL
PAHLLGDMWGRFWTNLYSLTVPFGQKPNIDVTDAM
VDQAWDAQRIFKEAEKFFVSVGLPNMTQGFWENSM
LTDPGNVQKAVCHPTAWDLGKGDFRILMCTKVTMD
DFLTAHNEMGNIQYDMAYAAQPFLLRNGANEGFHE
AVGEIMSLSAATPKHLKSIGLLSPDFQEDNETEIN
FLLKQALTIVGTLPFTYMLEKWRWMVFKGEIPKDQ
WMKKWWEMKREIVGVVEPVPHDETYCDPASLFHVS
NDYSFIRYYTRTLYQFQFQEALCQAAKHEGPLHKC
DISNSTEAGQKLFNMLRLGKSEPWTLALENVVGAK
NMNVRPLLNYFEPLFTWLKDQNKNSFVGWSTDWSP
YAD
SEQ ID NO: 4 Subset of ACE2
extracellular domain with H374N H374N
substitutions (residues 18-612)
QSTIEEQAKTFLDKFNHEAEDLFYQSSLASWNYNT
NITEENVQNMNNAGDKWSAFLKEQSTLAQMYPLQE
IQNLTVKLQLQALQQNGSSVLSEDKSKRLNTILNT
MSTIYSTGKVCNPDNPQECLLLEPGLNEIMANSLD
YNERLWAWESWRSEVGKQLRPLYEEYVVLKNEMAR
ANHYEDYGDYWRGDYEVNGVDGYDYSRGQLIEDVE
HTFEEIKPLYEHLHAYVRAKLMNAYPSYISPIGCL
PAHLLGDMWGRFWTNLYSLTVPFGQKPNIDVTDAM
VDQAWDAQRIFKEAEKFFVSVGLPNMTQGFWENSM
LTDPGNVQKAVCHPTAWDLGKGDFRILMCTKVTMD
DFLTAHNEMGNIQYDMAYAAQPFLLRNGANEGFHE
AVGEIMSLSAATPKHLKSIGLLSPDFQEDNETEIN
FLLKQALTIVGTLPFTYMLEKWRWMVFKGEIPKDQ
WMKKWWEMKREIVGVVEPVPHDETYCDPASLFHVS
NDYSFIRYYTRTLYQFQFQEALCQAAKHEGPLHKC
DISNSTEAGQKLFNMLRLGKSEPWTLALENVVGAK
NMNVRPLLNYFEPLFTWLKDQNKNSFVGWSTDWSP

TABLE 2
Hinges derived from IgG4 with S228P
substitution & Other linkers

SEQ ID NO: 5
VESKYGPPCPPCP
SEQ ID NO: 6
ESKYGPPCPPCP
SEQ ID NO: 7
SKYGPPCPPCP
SEQ ID NO: 8
KYGPPCPPCP
SEQ ID NO: 9
YGPPCPPCP
SEQ ID NO: 10
GPPCPPCP
SEQ ID NO: 11
PPCPPCP
SEQ ID NO: 12
PCPPCP
SEQ ID NO: 13
CPPCP
SEQ ID NO: 14
PPCP
SEQ ID NO: 15
PCP
SEQ ID NO: 52
(similar to SEQ ID NO: 5.
except first residue is G)
GESKYGPPCPPCP
SEQ ID NO: 53 (The G4AG4 linker)
GGGGAGGGG
SEQ ID NO: 54 (The G4AG4AP4 linker)
GGGGAGGGGAGGGG

TABLE 3
Hinges derived from IgG1

SEP ID NO: 16
EPKSCDKTHTCP
SEQ ID NO: 17
PKSCDKTHTCP
SEQ ID NO: 18
KSCDKTHTCP
SEQ IP NO: 19
SCDKTHTCP
SEQ IP NO: 20
CDKTHTCP
SEQ IP NO: 21
DKTHTCP
SEQ IP NO: 22
KTHTCP
SEQ IP NO: 23
THTCP
SEQ ID NO: 24
HTCP
SEQ ID NO: 25
TCP

TABLE 4
Hinges derived from IgG2

SEQ ID NO: 26
ERKCCVECPPCP
SEQ ID NO: 27
RKCCVECPPCP
SEQ ID NO: 28
KCCVECPPCP
SEQ ID NO: 29
CCVECPPCP
SEQ ID NO: 30
CVECPPCP
SEQ ID NO: 31
VECPPCP
SEQ ID NO: 32
ECPPCP

TABLE 5
Fc Domains

SEQ ID NO: 33 IgG4 Fc domain with
L235E substitution
APEFEGGPSVFLFPPKPKDTLMISRTPEVTCVVVD
VSQEDPEVQFNWYVDGVEVHNAKTKPREEQFNSTY
RVVSVLTVLHQDWLNGKEYKCKVSNKGLPSSIEKT
ISKAKGQPREPQVYTLPPSQEEMTKNQVSLTCLVK
GFYPSDIAVEWESNGQPENNYKTTPPVLDSDGSFF
LYSRLTVDKSRWQEGNVFSCSVMHEALHNHYTQKS
LSLSLGK
SEQ ID NO: 34 IgG4 Fc domain with
L235E and P329G substitutions
APEFEGGPSVFLFPPKPKDTLMISRTPEVTCVVVD
VSQEDPEVQFNWYVDGVEVHNAKTKPREEQFNSTY
RVVSVLTVLHQDWLNGKEYKCKVSNKGLGSSIEKT
ISKAKGQPREPQVYTLPPSQEEMTKNQVSLTCLVK
GFYPSDIAVEWESNGQPENNYKTTPPVLDSDGSFF
LYSRLTVDKSRWQEGNVFSCSVMHEALHNHYTQKS
LSLSLGK
SEQ ID NO: 35 IgG4 Fc domain with
IgG1 P329G substitution
APEFLGGPSVFLFPPKPKDTLMISRTPEVTCVVVD
VSQEDPEVQFNWYVDGVEVHNAKTKPREEQFNSTY
RVVSVLTVLHQDWLNGKEYKCKVSNKGLGSSIEKT
ISKAKGQPREPQVYTLPPSQEEMTKNQVSLTCLVK
GFYPSDIAVEWESNGQPENNYKTTPPVLDSDGSFF
LYSRLTVDKSRWQEGNVFSCSVMHEALHNHYTQKS
LSLSLGK
SEQ ID NO: 36 IgG4 Fc domain with
IgG1 P329A substitution
APEFLGGPSVFLFPPKPKDTLMISRTPEVTCVVVD
VSQEDPEVQFNWYVDGVEVHNAKTKPREEQFNSTY
RVVSVLTVLHQDWLNGKEYKCKVSNKGLASSIEKT
ISKAKGQPREPQVYTLPPSQEEMTKNQVSLTCLVK
GFYPSDIAVEWESNGQPENNYKTTPPVLDSDGSFF
LYSRLTVDKSRWQEGNVFSCSVMHEALHNHYTQKS
LSLSLGK
SEQ ID NO: 37 IgG1 with L234A and
L235A substitutions
PCPAPEAAGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALPAPI
EKTISKAKGQPREPQVYTLPPSREEMTKNQVSLTC
LVKGFYPSDIAVEWESNGQPENNYKTTPPVLDSDG
SFFLYSKLTVDKSRWQQGNVFSCSVMHEALHNHYT
QKSLSLSPGK
SEQ ID NO: 38 IgG1 with L234A,
L235A, and P329G substitutions
PCPAPEAAGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALGAPI
EKTISKAKGQPREPQVYTLPPSREEMTKNQVSLTC
LVKGFYPSDIAVEWESNGQPENNYKTTPPVLDSDG
SFFLYSKLTVDKSRWQQGNVFSCSVMHEALHNHYT
QKSLSLSPGK
SEQ ID NO: 39 IgG1 with N297A
substitution
PCPAPELLGGPSVFLFPPKPKDTLMISRTPEVTCV
VVDVSHEDPEVKFNWYVDGVEVHNAKTKPREEQYA
STYRVVSVLTVLHQDWLNGKEYKCKVSNKALPAPI
EKTISKAKGQPREPQVYTLPPSREEMTKNQVSLTC
LVKGFYPSDIAVEWESNGQPENNYKTTPPVLDSDG
SFFLYSKLTVDKSRWQQGNVFSCSVMHEALHNHYT
QKSLSLSPGK
SEQ ID NO: 40 IgG1 with N297D
substitution
PCPAPELLGGPSVFLFPPKPKDTLMISRTPEVTCV
VVDVSHEDPEVKFNWYVDGVEVHNAKTKPREEQYD
STYRVVSVLTVLHQDWLNGKEYKCKVSNKALPAPI
EKTISKAKGQPREPQVYTLPPSREEMTKNQVSLTC
LVKGFYPSDIAVEWESNGQPENNYKTTPPVLDSDG
SFFLYSKLTVDKSRWQQGNVFSCSVMHEALHNHYT
QKSLSLSPGK
SEQ ID NO: 41 IgG1 with P329G
substitution
PCPAPELLGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALGAPI
EKTISKAKGQPREPQVYTLPPSREEMTKNQVSLTC
LVKGFYPSDIAVEWESNGQPENNYKTTPPVLDSDG
SFFLYSKLTVDKSRWQQGNVFSCSVMHEALHNHYT
QKSLSLSPGK
SEQ ID NO: 42 IgG1 with P329A
substitution
PCPAPELLGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALAAPI
EKTISKAKGQPREPQVYTLPPSREEMTKNQVSLTC
LVKGFYPSDIAVEWESNGQPENNYKTTPPVLDSDG
SFFLYSKLTVDKSRWQQGNVFSCSVMHEALHNHYT
QKSLSLSPGK
SEQ ID NO: 55 IgG1 with L234A and
L235A substitutions (similar to
SEQ ID NO: 37, except has “DEL”
instead of “EEM”)
PCPAPEAAGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALPAPI
EKTISKAKGQPREPQVYTLPPSRDELTKNQVSLTC
LVKGFYPSDIAVEWESNGQPENNYKTTPPVLDSDG
SFFLYSKLTVDKSRWQQGNVFSCSVMHEALHNHYT
QKSLSLSPGK

TABLE 6
Reference IgG Fc and constant domains

```
SEQ ID NO: 43 IgG4 Fc domain
APEFLGGPSVFLFPPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK
SEQ ID NO: 44 IgG2 Fc domain
APPVAGPSVFLFPPKPKDTLMISRTPEVTCVVVDVSHEDPEVQFNWYVDGMEVHNAKTKPREEQFNSTFRVVS
VLTVVHQDWLNGKEYKCKVSNKGLPAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCSVMHEALHNHYTQKSLSLSPGK
SEQ ID NO: 45 Human IgG1 constant region (Eu numbering)
              127 137 147 157 167
ASTKGPSVFP LAPSSKSTSG GTAALGCLVK DYFPEPVTVS WNSGALTSGV
              177 187 197 207 217
HTFPAVLQSS GLYSLSSVVT VPSSSLGTQT YICNVNHKPS NTKVDKKVEP
              227 237 247 257 267
KSCDKTHTCP PCPAPELLGG PSVFLFPPKP KDTLMISRTP EVTCVVVDVS
              277 287 297 307 317
HEDPEVKFNW YVDGVEVHNA KTKPREEQYN STYRVVSVLT VLHQDWLNGK
              327 337 347 357 367
EYKCKVSNKA LPAPIEKTIS KAKGQPREPQ VYTLPPSRDE LTKNQVSLTC
              377 387 397 407 417
LVKGFYPSDI AVEWESNGQP ENNYKTTPPV LDSDGSFFLY SKLTVDKSRW
              427 437 447
QQGNVFSCSV MHEALHNHYT QKSLSLSPGK
SEQ ID NO: 46 Human IgG2 constant
              127 137 147 157 167
ASTKGPSVFP LAPCSRSTSE STAALGCLVK DYFPEPVTVS WNSGALTSGV
              177 187 197 207 217
HTFPAVLQSS GLYSLSSVVT VPSSNFGTQT YTCNVDHKPS NTKVDKTVER
      |*# 230 240 250 260 270
KCCVECPPCP APPVAGPSVF LFPPKPKDTL MISRTPEVTC VVVDVSHEDP
              280 290 300 310 320
EVQFNWYVDG VEVHNAKTKP REEQFNSTFR VVSVLTVVHQ DWLNGKEYKC
              330 340 350 360 370
KVSNKGLPAP IEKTISKTKG QPREPQVYTL PPSREEMTKN QVSLTCLVKG
              380 390 400 410 420
FYPSDISVEW ESNGQPENNY KTTPPMLDSD GSFFLYSKLT VDKSRWQQGN
              430 440 446
VFSCSVMHEA LHNHYTQKSL SLSPGK
| = V222, * = E224, # = C226
SEQ ID NO: 47 Human IgG4 constant region (Eu numbering)
              127 137 147 157 167
ASTKGPSVFP LAPCSRSTSE STAALGCLVK DYFPEPVTVS WNSGALTSGV
              177 187 197 207 217
HTFPAVLQSS GLYSLSSVVT VPSSSLGTKT YTCNVDHKPS NTKVDKRVES
  ||226 230 240 250 260 270
KYGPPCPSCP APEFLGGPSV FLFPPKPKDT LMISRTPEVT CVVVDVSQED
              280 290 300 310 320
PEVQFNWYVD GVEVHNAKTK PREEQFNSTY RVVSVLTVLH QDWLNGKEYK
              330 340 350 360 370
CKVSNKGLPS SIEKTISKAK GQPREPQVYT LPPSQEEMTK NQVSLTCLVK
              380 390 400 410 420
GFYPSDIAVE WESNGQPENN YKTTPPVLDS DGSFFLYSRL TVDKSRWQEG
              430 440 447
NVFSCSVMHE ALHNHYTQKS LSLSLGK
|| = These IgG4 hinge residues (Y & G) do not have an Eu number
```

TABLE 7
SEQ ID NO: 48 DF-COV-01 amino acid sequence
QSTIEEQAKTFLDKFNHEAEDLFYQSSLASWNYNTNITEENVQNMNNAGDKWSAFLKEQSTLAQMYPLQEIQN
LTVKLQLQALQQNGSSVLSEDKSKRLNTILNTMSTIYSTGKVCNPDNPQECLLLEPGLNEIMANSLDYNERLW
AWESWRSEVGKQLRPLYEEYVVLKNEMARANHYEDYGDYWRGDYEVNGVDGYDYSRGQLIEDVEHTFEEIKPL
YEHLHAYVRAKLMNAYPSYISPIGCLPAHLLGDMWGRFWTNLYSLTVPFGQKPNIDVTDAMVDQAWDAQRIFK
EAEKFFVSVGLPNMTQGFWENSMLTDPGNVQKAVCHPTAWDLGKGDFRILMCTKVTMDDFLTAHNEMGNIQYD
MAYAAQPFLLRNGANEGFHEAVGEIMSLSAATPKHLKSIGLLSPDFQEDNETEINFLLKQALTIVGTLPFTYM
LEKWRWMVFKGEIPKDQWMKKWWEMKREIVGVVEPVPHDETYCDPASLFHVSNDYSFIRYYTRTLYQFQFQEA
LCQAAKHEGPLHKCDISNSTEAGQKLFNMLRLGKSEQWTLALENVVGAKNMNVRPLLNYFEPLFTWLKDQNKN
SFVGWSTDWSPYADQSIKVRISLKSALGDKAYEWNDNEMYLFRSSVAYAMRQYFLKVKNQMILFGEEDVRVAN
LKPRISFNFFVTAPKNVSDIIPRTEVEKAIRMSRSRINDAFRLNDNSLEFLGIQPTLGPPNQPPVSGPPCPPC
PAPEFEGGPSVFLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALHNHYTQKSLSLSLGK
*SEQ ID NO: 60 ACE2 amino acid sequence
MSSSSWLLLSLVAVTAAQSTIEEQAKTFLDKFNHEAEDLFYQSSLASWNYNTNITEENVQNMNNAGDKWSAFL
KEQSTLAQMYPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVNGVDGYDYSR
GQLIEDVEHTFEEIKPLYEHLHAYVRAKLMNAYPSYISPIGCLPAHLLGDMWGRFWTNLYSLTVPFGQKPNID
VTDAMVDQAWDAQRIFKEAEKFFVSVGLPNMTQGFWENSMLTDPGNVQKAVCHPTAWDLGKGDFRILMCTKVT
MDDFLTAHHEMGHIQYDMAYAAQPFLLRNGANEGFHEAVGEIMSLSAATPKHLKSIGLLSPDFQEDNETEINF
LLKQALTIVGTLPFTYMLEKWRWMVFKGEIPKDQWMKKWWEMKREIVGVVEPVPHDETYCDPASLFHVSNDYS
FIRYYTRTLYQFQFQEALCQAAKHEGPLHKCDISNSTEAGQKLFNMLRLGKSEQWTLALENVVGAKNMNVRPL
LNYFEPLFTWLKDONKNSFVGWSTDWSPYADQSIKVRISLKSALGDKAYEWNDNEMYLFRSSVAYAMRQYFLK
VKNQMILFGEEDVRVANLKPRISFNFFVTAPKNVSDIIPRTEVEKAIRMSRSRINDAFRLNDNSLEFLGIQPT
LGPPNQPPVSIWLIVFGVVMGVIVVGIVILIFTGIRDRKKKNKARSGENPYASIDISKGENNPGFQNTDDVQT
SF
* A representative sequence of ACE2 is provided as SEQ ID NO: 60.
The aminoterminal leader is italicized and the ACE2 peptidase
domain (PD) (amino acids 18 through 615) is emboldened.
The portion of ACE2 carboxyterminal to amino acid 615
(616 through 768) has been termed the collectrin-like
domain (CLD) (Yan et al. (2020) Science 367:1444-1448).
This terminology relates to the fact that this region,
which includes the juxtamembrane, transmembrane, and
cytoplasmic domains of ACE2 share similarity with the
renal transmembrane protein collectrin (Hamming et al.
(2007) J. Pathol. 212:1-11). In particular, sequence
alignment demonstrates that amino acids 614 thorugh
the carboxyterminus of ACE2 share 47.8% identity with
amino acids 21 through 241 of collectrin (Zhang et al.
(2001) J. Biol. Chem. 276:17132-17139). In the
representative SEQ ID NO: 60, the collectrin-like
domain (CLD) is not emboldened. The juxtamembrane
(extracellular) portion of the CLD, amino acids 616
through 740 is underlined and the transmembrane and
cytoplasmic portions of the CLD are not underlined.
SEQ ID NO: 49 DF-COV-02 amino acid sequence
QSTIEEQAKTFLDKFNHEAEDLFYQSSLASWNYNTNITEENVQNMNNAGDKWSAFLKEQSTLAQMYPLQEIQN
LTVKLQLQALQQNGSSVLSEDKSKRLNTILNTMSTIYSTGKVCNPDNPQECLLLEPGLNEIMANSLDYNERLW
AWESWRSEVGKQLRPLYEEYVVLKNEMARANHYEDYGDYWRGDYEVNGVDGYDYSRGQLIEDVEHTFEEIKPL
YEHLHAYVRAKLMNAYPSYISPIGCLPAHLLGDMWGRFWTNLYSLTVPFGQKPNIDVTDAMVDQAWDAQRIFK
EAEKFFVSVGLPNMTQGFWENSMLTDPGNVQKAVCHPTAWDLGKGDFRILMCTKVTMDDFLTAHNEMGNIQYD
MAYAAQPFLLRNGANEGFHEAVGEIMSLSAATPKHLKSIGLLSPDFQEDNETEINFLLKQALTIVGTLPFTYM
LEKWRWMVFKGEIPKDQWMKKWWEMKREIVGVVEPVPHDETYCDPASLFHVSNDYSFIRYYTRTLYQFQFQEA
LCQAAKHEGPLHKCDISNSTEAGQKLFNMLRLGKSEQWTLALENVVGAKNMNVRPLLNYFEPLFTWLKDQNKN
SFVGWSTDWSPGESKYGPPCPPCPAPEFEGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLTVDKSRWQEGNVFSC
SVMHEALHNHYTQKSLSLSLGK
SEQ ID NO: 56 DF-COV-03 amino acid sequence ([ACE2(18-615)-NN]-[G4AG4]-
[IgG1 hinge]-[hIgG1(LALA) Fc]) [IgG1 hinge is bold and italicized]
QSTIEEQAKTFLDKFNHEAEDLFYQSSLASWNYNTNITEENVONMNNAGDKWSAFLKEQSTLAQMYPLQEIQN
LTVKLQLQALQQNGSSVLSEDKSKRLNTILNTMSTIYSTGKVCNPDNPQECLLLEPGLNEIMANSLDYNERLW
AWESWRSEVGKQLRPLYEEYVVLKNEMARANHYEDYGDYWRGDYEVNGVDGYDYSRGQLIEDVEHTFEEIKPL
YEHLHAYVRAKLMNAYPSYISPIGCLPAHLLGDMWGRFWTNLYSLTVPFGQKPNIDVTDAMVDQAWDAQRIFK
EAEKFFVSVGLPNMTQGFWENSMLTDPGNVQKAVCHPTAWDLGKGDFRILMCTKVTMDDFLTAHNEMGNIQYD
MAYAAQPFLLRNGANEGFHEAVGEIMSLSAATPKHLKSIGLLSPDFQEDNETEINFLLKQALTIVGTLPFTYM
LEKWRWMVFKGEIPKDQWMKKWWEMKREIVGVVEPVPHDETYCDPASLFHVSNDYSFIRYYTRTLYQFQFQEA
LCQAAKHEGPLHKCDISNSTEAGQKLFNMLRLGKSEQWTLALENVVGAKNMNVRPLLNYFEPLFTWLKDQNKN
SFVGWSTDWSPYADGGGGAGGGGDKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
SEQ ID NO: 57 DF-COV-04 amino acid sequence ([IgG1 hinge]-[hIgG1(LALA) Fc]-
[G4AG4AG4]-[ACE2(18-612)-NN]) [IgG1 hinge is bold and italicized]
DKTHTCPPCPAPEAAGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGKGGGGAGGGGAGGGGQSTIEEQAKTFLDKFNHEAEDLFYQSSLASWNYNTNITEENVQNMNNAGDK
WSAFLKEQSTLAQMYPLQEIQNLTVKLQLQALQQNGSSVLSEDKSKRLNTILNTMSTIYSTGKVCNPDNPQEC
LLLEPGLNEIMANSLDYNERLWAWESWRSEVGKQLRPLYEEYVVLKNEMARANHYEDYGDYWRGDYEVNGVDG
YDYSRGQLIEDVEHTFEEIKPLYEHLHAYVRAKLMNAYPSYISPIGCLPAHLLGDMWGRFWTNLYSLTVPFGQ
KPNIDVTDAMVDQAWDAQRIFKEAEKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMSLSAATPKHLKSIGLLSPDFQEDNE
TEINFLLKQALTIVGTLPFTYMLEKWRWMVFKGEIPKDQWMKKWWEMKREIVGVVEPVPHDETYCDPASLFHV
SNDYSFIRYYTRTLYQFQFQEALCQAAKHEGPLHKCDISNSTEAGQKLFNMLRLGKSEQWTLALENVVGAKNM
NVRPLLNYFEPLFTWLKDQNKNSFVGWSTDWSP

TABLE 8
DF-COV nucleic acid sequences

SEQ ID NO: 50 full DF-COV-01
nucleotide sequence
ATGGAATGGAGCTGGGTCTTTCTCTTCTTCCTGTC
AGTAACGACTGGTGTCCACTCCCAGTCAACAATCG
AGGAGCAGGCAAAGACCTTCCTGGACAAGTTTAAC
CACGAAGCGGAGGACCTGTTCTACCAGAGCAGCCT
GGCGAGCTGGAATTACAACACCAACATCACCGAGG
AGAACGTGCAGAACATGAATAACGCTGGCGACAAG
TGGAGCGCCTTCTTGAAGGAGCAATCCACCCTGGC
CCAGATGTACCCGCTGCAAGAGATACAGAACCTGA
CTGTGAAGCTGCAACTGCAGGCCCTGCAGCAGAAC
GGCTCCAGCGTGCTGAGCGAAGACAAGAGCAAAAG
GCTGAATACCATATTGAACACGATGAGCACCATCT
ACAGCACCGGCAAAGTATGCAACCCCGACAACCCC
CAAGAGTGTCTGTTGTTGGAACCCGGCCTGAACGA
GATTATGGCGAACTCCCTGGACTACAACGAACGCC
TGTGGGCATGGGAGTCTTGGAGGTCAGAGGTTGGC
AAGCAACTGAGGCCGTTGTACGAAGAGTACGTGGT
GCTGAAGAACGAAATGGCACGGGCCAATCATTATG
AGGACTACGGTGATTATTGGAGGGGCGACTACGAG
GTGAACGGCGTGGACGGCTACGACTATAGCAGGGG
CCAACTTATAGAGGACGTAGAGCACACTTTTGAGG
AAATCAAACCCCTGTACGAGCACTTGCATGCCTAC
GTACGCGCCAAACTGATGAATGCGTACCCCAGCTA
CATCAGCCCCATCGGCTGCCTGCCCGCACACCTGC
TCGGCGACATGTGGGGCCGATTCTGGACCAATCTG
TATAGCCTCACCGTGCCCTTTGGCCAGAAGCCGAA
CATAGATGTGACGGATGCTATGGTAGACCAGGCGT
GGGATGCACAGCGCATCTTCAAGGAGGCCGAGAAG
TTCTTCGTGAGCGTCGGCTTGCCCAACATGACCCA
GGGTTTCTGGGAGAACTCAATGCTGACTGACCCCG
GCAACGTACAGAAAGCCGTATGCCACCCAACTGCT
TGGGACCTGGGTAAGGGAGACTTCCGGATCCTCAT
GTGTACCAAGGTGACCATGGATGACTTTCTCACCG
CCCACAATGAAATGGGCAACATCCAGTACGATATG
GCCTACGCAGCGCAGCCATTCCTTCTGAGGAACGG
TGCCAACGAGGGCTTCCATGAGGCAGTCGGAGAGA
TCATGAGCCTGTCAGCGGCGACCCCTAAACATCTG
AAGAGCATCGGGCTGCTGTCTCCGGACTTTCAAGA
GGACAATGAGACTGAGATCAACTTCCTCCTGAAAC
AGGCGCTGACCATTGTAGGCACCTTGCCCTTCACC
TACATGCTGGAGAAGTGGCGATGGATGGTGTTTAA
GGGCGAGATCCCGAAGGACCAGTGGATGAAAAAGT
GGTGGGAGATGAAGAGGGAGATCGTCGGCGTAGTT
GAGCCGGTCCCCCACGACGAAACCTACTGCGATCC
TGCCAGCCTGTTCCACGTGAGCAACGATTACAGTT
TTATCAGGTACTACACCAGGACCCTTTACCAATTT
CAGTTCCAGGAGGCACTGTGCCAGGCCGCCAAGCA
CGAAGGCCCCCTGCACAAGTGCGACATCAGCAACA
GCACCGAGGCGGGTCAAAAGCTGTTTAACATGCTG
AGGCTGGGCAAGAGCGAGCCGTGGACCCTGGCCCT
GGAAAACGTTGTGGGCGCAAAAAACATGAACGTGA
GGCCCCTGCTGAACTACTTCGAGCCCCTGTTCACC
TGGCTGAAGGACCAGAACAAGAACAGTTTCGTGGG
CTGGAGTACCGATTGGAGTCCCTATGCCGACCAGA
GCATCAAGGTGAGGATTAGCCTCAAGAGCGCCCTG
GGCGACAAGGCCTATGAATGGAACGACAACGAGAT
GTACCTGTTCAGGTCAAGCGTGGCCTACGCCATGA
GGCAGTACTTCCTGAAAGTGAAGAACCAGATGATA
CTGTTCGGCGAGGAGGACGTGAGGGTGGCCAACCT
GAAGCCCAGGATATCATTCAATTTCTTCGTGACCG
CTCCCAAGAACGTGAGCGACATCATCCCCAGGACC
GAGGTGGAGAAGGCCATCAGGATGAGCCGCAGCAG
GATTAACGACGCCTTCAGGCTGAACGATAACAGCC
TGGAGTTCCTTGGCATCCAGCCAACCCTGGGACCA
CCCAACCAGCCTCCCGTTAGCGGACCCCCCTGTCC
TCCTTGCCCTGCTCCTGAATTTGAGGGAGGCCCCT
CCGTCTTCCTGTTTCCCCCCAAGCCCAAGGACACC
CTGATGATCTCCCGGACACCCGAAGTCACCTGCGT
CGTGGTGGATGTCAGCCAGGAAGATCCCGAGGTGC
AGTTCAACTGGTACGTGGACGGAGTGGAGGTGCAT
AACGCCAAAACCAAGCCCAGGGAAGAGCAGTTCAA
CAGCACCTATCGGGTCGTGTCCGTGCTCACCGTCC
TGCATCAGGATTGGCTCAACGGCAAGGAGTACAAG
TGCAAGGTGTCCAACAAGGGCCTGCCCTCCTCCAT
CGAGAAGACCATCTCCAAGGCTAAGGGCCAACCTC
GGGAGCCCCAAGTGTATACCCTCCCTCCCAGCCAG
GAGGAGATGACCAAGAATCAAGTGAGCCTGACCTG
CCTCGTGAAGGGATTTTACCCCTCCGACATCGCTG
TGGAATGGGAAAGCAATGGCCAACCTGAGAACAAC
TACAAGACCACACCCCCCGTGCTGGACTCCGATGG
CTCCTTCTTCCTGTACAGCAGGCTGACCGTGGACA
AATCCCGGTGGCAAGAGGGAAACGTGTTCAGCTGC
TCCGTGATGCACGAGGCTCTCCACAACCACTACAC
CCAGAAGAGCCTCTCCCTGAGCCTCGGCAAGTAA
SEQ ID NO: 51 full DF-COV-02
nucleotide sequence
ATGGAATGGAGCTGGGTCTTTCTCTTCTTCCTGTC
AGTAACGACTGGTGTCCACTCCCAGTCAACAATCG
AGGAGCAGGCAAAGACCTTCCTGGACAAGTTTAAC
CACGAAGCGGAGGACCTGTTCTACCAGAGCAGCCT
GGCGAGCTGGAATTACAACACCAACATCACCGAGG
AGAACGTGCAGAACATGAATAACGCTGGCGACAAG
TGGAGCGCCTTCTTGAAGGAGCAATCCACCCTGGC
CCAGATGTACCCGCTGCAAGAGATACAGAACCTGA
CTGTGAAGCTGCAACTGCAGGCCCTGCAGCAGAAC
GGCTCCAGCGTGCTGAGCGAAGACAAGAGCAAAAG
GCTGAATACCATATTGAACACGATGAGCACCATCT
ACAGCACCGGCAAAGTATGCAACCCCGACAACCCC
CAAGAGTGTCTGTTGTTGGAACCCGGCCTGAACGA
GATTATGGCGAACTCCCTGGACTACAACGAACGCC
TGTGGGCATGGGAGTCTTGGAGGTCAGAGGTTGGC
AAGCAACTGAGGCCGTTGTACGAAGAGTACGTGGT
GCTGAAGAACGAAATGGCACGGGCCAATCATTATG
AGGACTACGGTGATTATTGGAGGGGCGACTACGAG
GTGAACGGCGTGGACGGCTACGACTATAGCAGGGG
CCAACTTATAGAGGACGTAGAGCACACTTTTGAGG
AAATCAAACCCCTGTACGAGCACTTGCATGCCTAC
GTACGCGCCAAACTGATGAATGCGTACCCCAGCTA
CATCAGCCCCATCGGCTGCCTGCCCGCACACCTGC
TCGGCGACATGTGGGGCCGATTCTGGACCAATCTG
TATAGCCTCACCGTGCCCTTTGGCCAGAAGCCGAA
CATAGATGTGACGGATGCTATGGTAGACCAGGCGT
GGGATGCACAGCGCATCTTCAAGGAGGCCGAGAAG
TTCTTCGTGAGCGTCGGCTTGCCCAACATGACCCA
GGGTTTCTGGGAGAACTCAATGCTGACTGACCCCG
GCAACGTACAGAAAGCCGTATGCCACCCAACTGCT
TGGGACCTGGGTAAGGGAGACTTCCGGATCCTCAT
GTGTACCAAGGTGACCATGGATGACTTTCTCACCG
CCCACAATGAAATGGGCAACATCCAGTACGATATG
GCCTACGCAGCGCAGCCATTCCTTCTGAGGAACGG
TGCCAACGAGGGCTTCCATGAGGCAGTCGGAGAGA
TCATGAGCCTGTCAGCGGCGACCCCTAAACATCTG
AAGAGCATCGGGCTGCTGTCTCCGGACTTTCAAGA
GGACAATGAGACTGAGATCAACTTCCTCCTGAAAC
AGGCGCTGACCATTGTAGGCACCTTGCCCTTCACC
TACATGCTGGAGAAGTGGCGATGGATGGTGTTTAA
GGGCGAGATCCCGAAGGACCAGTGGATGAAAAAGT
GGTGGGAGATGAAGAGGGAGATCGTCGGCGTAGTT
GAGCCGGTCCCCCACGACGAAACCTACTGCGATCC
TGCCAGCCTGTTCCACGTGAGCAACGATTACAGTT
TTATCAGGTACTACACCAGGACCCTTTACCAATTT
CAGTTCCAGGAGGCACTGTGCCAGGCCGCCAAGCA
CGAAGGCCCCCTGCACAAGTGCGACATCAGCAACA
GCACCGAGGCGGGTCAAAAGCTGTTTAACATGCTG
AGGCTGGGCAAGAGCGAGCCGTGGACCCTGGCCCT
GGAAAACGTTGTGGGCGCAAAAAACATGAACGTGA
GGCCCCTGCTGAACTACTTCGAGCCCCTGTTCACC
TGGCTGAAGGACCAGAACAAGAACAGTTTCGTGGG
CTGGAGTACCGATTGGAGTCCCGGTGAATCCAAGT
ATGGACCCCCCTGTCCTCCTTGCCCTGCTCCTGAA
TTTGAGGGAGGCCCCTCCGTCTTCCTGTTTCCCCC
CAAGCCCAAGGACACCCTGATGATCTCCCGGACAC
CCGAAGTCACCTGCGTCGTGGTGGATGTCAGCCAG
GAAGATCCCGAGGTGCAGTTCAACTGGTACGTGGA
CGGAGTGGAGGTGCATAACGCCAAAACCAAGCCCA
GGGAAGAGCAGTTCAACAGCACCTATCGGGTCGTG
TCCGTGCTCACCGTCCTGCATCAGGATTGGCTCAA
CGGCAAGGAGTACAAGTGCAAGGTGTCCAACAAGG
GCCTGCCCTCCTCCATCGAGAAGACCATCTCCAAG
GCTAAGGGCCAACCTCGGGAGCCCCAAGTGTATAC
CCTCCCTCCCAGCCAGGAGGAGATGACCAAGAATC
AAGTGAGCCTGACCTGCCTCGTGAAGGGATTTTAC
CCCTCCGACATCGCTGTGGAATGGGAAAGCAATGG
CCAACCTGAGAACAACTACAAGACCACACCCCCCG
TGCTGGACTCCGATGGCTCCTTCTTCCTGTACAGC
AGGCTGACCGTGGACAAATCCCGGTGGCAAGAGGG
AAACGTGTTCAGCTGCTCCGTGATGCACGAGGCTC
TCCACAACCACTACACCCAGAAGAGCCTCTCCCTG
AGCCTCGGCAAGTAA
SEQ ID NO: 58 full DF-COV-03
nucleotide sequence
ATGGAATGGAGCTGGGTCTTTCTCTTCTTCCTGTC
AGTAACGACTGGTGTCCACTCCCAGTCAACAATCG
AGGAGCAGGCAAAGACCTTCCTGGACAAGTTTAAC
CACGAAGCGGAGGACCTGTTCTACCAGAGCAGCCT
GGCGAGCTGGAATTACAACACCAACATCACCGAGG
AGAACGTGCAGAACATGAATAACGCTGGCGACAAG
TGGAGCGCCTTCTTGAAGGAGCAATCCACCCTGGC
CCAGATGTACCCGCTGCAAGAGATACAGAACCTGA
CTGTGAAGCTGCAACTGCAGGCCCTGCAGCAGAAC
GGCTCCAGCGTGCTGAGCGAAGACAAGAGCAAAAG
GCTGAATACCATATTGAACACGATGAGCACCATCT
ACAGCACCGGCAAAGTATGCAACCCCGACAACCCC
CAAGAGTGTCTGTTGTTGGAACCCGGCCTGAACGA
GATTATGGCGAACTCCCTGGACTACAACGAACGCC
TGTGGGCATGGGAGTCTTGGAGGTCAGAGGTTGGC
AAGCAACTGAGGCCGTTGTACGAAGAGTACGTGGT
GCTGAAGAACGAAATGGCACGGGCCAATCATTATG
AGGACTACGGTGATTATTGGAGGGGCGACTACGAG
GTGAACGGCGTGGACGGCTACGACTATAGCAGGGG
CCAACTTATAGAGGACGTAGAGCACACTTTTGAGG
AAATCAAACCCCTGTACGAGCACTTGCATGCCTAC
GTACGCGCCAAACTGATGAATGCGTACCCCAGCTA
CATCAGCCCCATCGGCTGCCTGCCCGCACACCTGC
TCGGCGACATGTGGGGCCGATTCTGGACCAATCTG
TATAGCCTCACCGTGCCCTTTGGCCAGAAGCCGAA
CATAGATGTGACGGATGCTATGGTAGACCAGGCGT
GGGATGCACAGCGCATCTTCAAGGAGGCCGAGAAG
TTCTTCGTGAGCGTCGGCTTGCCCAACATGACCCA
GGGTTTCTGGGAGAACTCAATGCTGACTGACCCCG
GCAACGTACAGAAAGCCGTATGCCACCCAACTGCT
TGGGACCTGGGTAAGGGAGACTTCCGGATCCTCAT
GTGTACCAAGGTGACCATGGATGACTTTCTCACCG
CCCACAATGAAATGGGCAACATCCAGTACGATATG
GCCTACGCAGCGCAGCCATTCCTTCTGAGGAACGG
TGCCAACGAGGGCTTCCATGAGGCAGTCGGAGAGA
TCATGAGCCTGTCAGCGGCGACCCCTAAACATCTG
AAGAGCATCGGGCTGCTGTCTCCGGACTTTCAAGA
GGACAATGAGACTGAGATCAACTTCCTCCTGAAAC
AGGCGCTGACCATTGTAGGCACCTTGCCCTTCACC
TACATGCTGGAGAAGTGGCGATGGATGGTGTTTAA
GGGCGAGATCCCGAAGGACCAGTGGATGAAAAAGT
GGTGGGAGATGAAGAGGGAGATCGTCGGCGTAGTT
GAGCCGGTCCCCCACGACGAAACCTACTGCGATCC
TGCCAGCCTGTTCCACGTGAGCAACGATTACAGTT
TTATCAGGTACTACACCAGGACCCTTTACCAATTT
CAGTTCCAGGAGGCACTGTGCCAGGCCGCCAAGCA
CGAAGGCCCCCTGCACAAGTGCGACATCAGCAACA
GCACCGAGGCGGGTCAAAAGCTGTTTAACATGCTG
AGGCTGGGCAAGAGCGAGCCGTGGACCCTGGCCCT
GGAAAACGTTGTGGGCGCAAAAAACATGAACGTGA
GGCCCCTGCTGAACTACTTCGAGCCCCTGTTCACC
TGGCTGAAGGACCAGAACAAGAACAGTTTCGTGGG
CTGGAGTACCGATTGGAGTCCCTATGCCGACGGCG
GAGGTGGTGCAGGAGGCGGTGGAGACAAAACTCAC
ACATGCCCACCGTGCCCAGCACCTGAAGCCGCAGG
GGGACCGTCAGTCTTCCTCTTCCCCCCAAAACCCA
AGGACACCCTCATGATCTCCCGGACCCCTGAGGTC
ACATGCGTGGTGGTGGACGTGAGCCACGAAGACCC
TGAGGTCAAGTTCAACTGGTACGTGGACGGCGTGG
AGGTGCATAATGCCAAGACAAAGCCGCGGGAGGAG
CAGTACAACAGCACGTACCGTGTGGTCAGCGTCCT
CACCGTCCTGCACCAGGACTGGCTGAATGGCAAGG
AGTACAAGTGCAAGGTCTCCAACAAAGCCCTCCCA
GCCCCCATCGAGAAAACCATCTCCAAAGCCAAAGG
GCAGCCCCGAGAACCACAGGTGTACACCCTGCCCC
CATCCCGGGATGAGCTGACCAAGAACCAGGTCAGC
CTGACCTGCCTGGTCAAAGGCTTCTATCCCAGCGA
CATCGCCGTGGAGTGGGAGAGCAATGGGCAGCCGG
AGAACAACTACAAGACCACGCCTCCCGTGCTGGAC
TCCGACGGCTCCTTCTTCCTCTACAGCAAGCTCAC
CGTGGACAAGAGCAGGTGGCAGCAGGGGAACGTCT
TCTCATGCTCCGTGATGCATGAGGCTCTGCACAAC
CACTACACGCAGAAGAGCCTCTCCCTGTCTCCGGG
TAAATGA
SEQ ID NO: 59 full DF-COV-04
nucleotide sequence
ATGGAATGGAGCTGGGTCTTTCTCTTCTTCCTGTC
AGTAACGACTGGTGTCCACTCCGACAAAACTCACA
CATGCCCACCGTGCCCAGCACCTGAAGCCGCAGGG
GGACCGTCAGTCTTCCTCTTCCCCCCAAAACCCAA
GGACACCCTCATGATCTCCCGGACCCCTGAGGTCA
CATGCGTGGTGGTGGACGTGAGCCACGAAGACCCT
GAGGTCAAGTTCAACTGGTACGTGGACGGCGTGGA
GGTGCATAATGCCAAGACAAAGCCGCGGGAGGAGC
AGTACAACAGCACGTACCGTGTGGTCAGCGTCCTC
ACCGTCCTGCACCAGGACTGGCTGAATGGCAAGGA
GTACAAGTGCAAGGTCTCCAACAAAGCCCTCCCAG
CCCCCATCGAGAAAACCATCTCCAAAGCCAAAGGG
CAGCCCCGAGAACCACAGGTGTACACCCTGCCCCC
ATCCCGGGATGAGCTGACCAAGAACCAGGTCAGCC
TGACCTGCCTGGTCAAAGGCTTCTATCCCAGCGAC
ATCGCCGTGGAGTGGGAGAGCAATGGGCAGCCGGA
GAACAACTACAAGACCACGCCTCCCGTGCTGGACT
CCGACGGCTCCTTCTTCCTCTACAGCAAGCTCACC
GTGGACAAGAGCAGGTGGCAGCAGGGGAACGTCTT
CTCATGCTCCGTGATGCATGAGGCTCTGCACAACC
ACTACACGCAGAAGAGCCTCTCCCTGTCTCCGGGT
AAAGGCGGAGGTGGTGCAGGAGGCGGTGGAGCCGG
TGGCGGAGGACAGTCAACAATCGAGGAGCAGGCAA
AGACCTTCCTGGACAAGTTTAACCACGAAGCGGAG
GACCTGTTCTACCAGAGCAGCCTGGCGAGCTGGAA
TTACAACACCAACATCACCGAGGAGAACGTGCAGA
ACATGAATAACGCTGGCGACAAGTGGAGCGCCTTC
TTGAAGGAGCAATCCACCCTGGCCCAGATGTACCC
GCTGCAAGAGATACAGAACCTGACTGTGAAGCTGC
AACTGCAGGCCCTGCAGCAGAACGGCTCCAGCGTG
CTGAGCGAAGACAAGAGCAAAAGGCTGAATACCAT
ATTGAACACGATGAGCACCATCTACAGCACCGGCA
AAGTATGCAACCCCGACAACCCCCAAGAGTGTCTG
TTGTTGGAACCCGGCCTGAACGAGATTATGGCGAA
CTCCCTGGACTACAACGAACGCCTGTGGGCATGGG
AGTCTTGGAGGTCAGAGGTTGGCAAGCAACTGAGG
CCGTTGTACGAAGAGTACGTGGTGCTGAAGAACGA
AATGGCACGGGCCAATCATTATGAGGACTACGGTG
ATTATTGGAGGGGCGACTACGAGGTGAACGGCGTG
GACGGCTACGACTATAGCAGGGGCCAACTTATAGA
GGACGTAGAGCACACTTTTGAGGAAATCAAACCCC
TGTACGAGCACTTGCATGCCTACGTACGCGCCAAA
CTGATGAATGCGTACCCCAGCTACATCAGCCCCAT
CGGCTGCCTGCCCGCACACCTGCTCGGCGACATGT
GGGGCCGATTCTGGACCAATCTGTATAGCCTCACC
GTGCCCTTTGGCCAGAAGCCGAACATAGATGTGAC
GGATGCTATGGTAGACCAGGCGTGGGATGCACAGC
GCATCTTCAAGGAGGCCGAGAAGTTCTTCGTGAGC
GTCGGCTTGCCCAACATGACCCAGGGTTTCTGGGA
GAACTCAATGCTGACTGACCCCGGCAACGTACAGA
AAGCCGTATGCCACCCAACTGCTTGGGACCTGGGT
AAGGGAGACTTCCGGATCCTCATGTGTACCAAGGT
GACCATGGATGACTTTCTCACCGCCCACAATGAAA
TGGGCAACATCCAGTACGATATGGCCTACGCAGCG
CAGCCATTCCTTCTGAGGAACGGTGCCAACGAGGG
CTTCCATGAGGCAGTCGGAGAGATCATGAGCCTGT
CAGCGGCGACCCCTAAACATCTGAAGAGCATCGGG
CTGCTGTCTCCGGACTTTCAAGAGGACAATGAGAC
TGAGATCAACTTCCTCCTGAAACAGGCGCTGACCA
TTGTAGGCACCTTGCCCTTCACCTACATGCTGGAG
AAGTGGCGATGGATGGTGTTTAAGGGCGAGATCCC
GAAGGACCAGTGGATGAAAAAGTGGTGGGAGATGA
AGAGGGAGATCGTCGGCGTAGTTGAGCCGGTCCCC
CACGACGAAACCTACTGCGATCCTGCCAGCCTGTT
CCACGTGAGCAACGATTACAGTTTTATCAGGTACT
ACACCAGGACCCTTTACCAATTTCAGTTCCAGGAG
GCACTGTGCCAGGCCGCCAAGCACGAAGGCCCCCT
GCACAAGTGCGACATCAGCAACAGCACCGAGGCGG
GTCAAAAGCTGTTTAACATGCTGAGGCTGGGCAAG
AGCGAGCCGTGGACCCTGGCCCTGGAAAACGTTGT
GGGCGCAAAAAACATGAACGTGAGGCCCCTGCTGA
ACTACTTCGAGCCCCTGTTCACCTGGCTGAAGGAC
CAGAACAAGAACAGTTTCGTGGGCTGGAGTACCGA
TTGGAGTCCCTGA

Included in Tables 1-7 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1 or 5-7, or a portion thereof. Such polypeptides can have a function of the full-length polypeptide as described further herein.
Included in Table 8 are RNA nucleic acid molecules (e.g., thymines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 8, or a portion thereof. Such nucleic acid molecules can have a function of the full-length nucleic acid as described further herein.
Included in Table 6 are SEQ ID NOs: 45-47 that are full-length IgG1, IgG2, and IgG4 sequences numbered using the “Eu” numbering system, which is also used for the Fc domain substitutions described herein. The numbering is the same for the CH1 domain of IgG1, IgG2, and IgG4. But, because the hinge regions are of different lengths, the numbering within the hinge, CH2, and CH3 domains is different in IgG1, IgG2, and IgG4. The Eu numbering system can be used in place of the IMGT and Kabat systems (see www.imgt.org/IMGTScientificChart/Numbering/Hu_IGHGnber.html and Edelman et al. (1969) Proc. Natl. Acad. USA, 63: 78-85).

### II. Agents and Compositions

a. Nucleic Acids

One aspect encompassed by the present invention pertains to nucleic acid molecules that encode an ACE2-Fc fusion polypeptide that binds a coronavirus (e.g., SARS-CoV-2). In one embodiment, a nucleic acid of the invention encodes an ACE2-Fc fusion polypeptide that binds a coronavirus (e.g., SARS-CoV-2), thereby competitively inhibiting the virus from binding to endogenous ACE2 expressed on a cell surface, wherein said polypeptide comprises 1) an amino acid sequence having at least 70%, 80%, 90% or greater identity to an ACE2 extracellular domain, or fragment thereof, amino acid sequence of any one of SEQ ID NOs: 1-4; 2) a Fc domain, or fragment thereof, polypeptide that has an amino acid sequence having at least 70%, 80%, 90% or greater identity to an Fc domain, or fragment thereof, amino acid sequence of any one of SEQ ID NOs: 33-42 or 55; and 3) an intervening hinge polypeptide between the ACE2 extracellular domain, or fragment thereof, and the Fc domain, or fragment thereof, wherein the hinge polypeptide has an amino acid sequence of any one of SEQ ID NOs: 5-32 or 52.

As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded DNA.

A nucleic acid molecule encompassed by the present invention encodes an ACE2-Fc fusion polypeptide encompassed by the present invention, wherein the ACE2-Fc fusion polypeptide comprises an ACE2 extracellular domain, or fragment thereof, a hinge polypeptide, and an Fc domain, or fragment thereof. The ACE2 extracellular domain, or fragment thereof, encoded by the nucleic acid has an amino acid sequence shown in Table 1, or an amino acid sequence which is at least about 50%, at least about 60%, at least about 70%, at least about 80%, at least about 90%, or at least about 95% or more (e.g., about 98%) homologous to the amino acid sequence shown in Table 1, or a portion thereof (i.e., 100, 200, 300, 400, 450, 500, or more amino acid residues), and wherein the nucleic acid wherein the polypeptide encoded by the nucleic acid molecule further comprises amino acid substitutions at positions corresponding to H374N and H378N. In some embodiments, the nucleic acid wherein the polypeptide encoded by the nucleic acid molecule further comprises one or more amino acid substitutions at positions corresponding to R273Q, H345A, H345L, H505A, and H505L. In some embodiments, the nucleic acid molecule further comprises amino acid substitutions at positions corresponding to R169Q, W271Q, and K481Q. The above amino acid substitutions are relative to the full length ACE2 polypeptide. In some embodiments, the nucleic acid encodes an ACE2 extracellular domain comprising amino acids 18-600, 18-615, 18-708, or 18-740 of the full length ACE2 polypeptide.

The Fc domain, or fragment thereof, encoded by the nucleic acid molecule encompassed by the present invention has an amino acid sequence shown in Table 5, or an amino acid sequence which is at least about 50%, at least about 60%, at least about 70%, at least about 80%, at least about 90%, or at least about 95% or more (e.g., about 98%) homologous to the amino acid sequence shown in Table 5, or a portion thereof (i.e., 100, 200, 300, 400, 450, 500, or more amino acid residues). The Fc domain, or fragment thereof, may comprise variants in its nucleic acid sequence that result in an Fc polypeptide with attenuated Fc receptor (i.e., FcγIIaR) binding. For example, a human IgG4 Fc domain, or fragment thereof, comprising the S228P and/or L235E point substitutions can attenuate Fc binding to a FcγR (e.g., a FcγIIa receptor). In one embodiment, the Fc domain fragment comprises the S228 or the L235E amino acid substitution. In some embodiments, the Fc domain fragment comprises an S228P and an L235E amino acid substitution. Other substitutions that can attenuate or eliminate Fc domain, or a fragment thereof, binding to a FcγR (e.g., a FcγIIa receptor) include L234A, L235A, N297X, and/or P329G mutants in IgG1 Fc domains, or fragment thereof. Thus, in some embodiments, the Fc domain fragment comprises L234A and L235A amino acid substitutions. In some embodiments, the Fc domain fragment comprises an L234A, L235A, N297A, N297D, and/or P329G amino acid substitution. In still other embodiments, the Fc domain fragment comprises L234A and N297A or N297D amino acid substitutions. In still other embodiments, the Fc domain fragment comprises L235A and N297A or N297D amino acid substitutions. In some embodiments, the Fc domain fragment comprises L234A, L235A, and a P329G amino acid substitutions. The amino acid substitutions are relative to full-length IgG Fc domain. In some embodiments, the hinge polypeptide may comprise a mutation that attenuates Fab-arm exchange. For example, in some embodiments, the hinge region comprises an amino acid sequence from Table 2, each of which comprise the S228P substitution. Generally, S228P substitution inhibits Fab-arm exchange, which occurs naturally in IgG4 antibodies and S228P does not have an effect on Fc gamma receptor binding. The IgG4 Fc domain has a lower affinity for Fc gamma receptors than IgG1 and the L235E substitution further lowers this affinity. The Fc domain, or fragment thereof, can further comprise an additional substitution (e.g., the L235E substitution) to attenuate or eliminate Fc domain, or fragment thereof, binding to a Fc receptor.

Nucleic acid molecules encoding other ACE2-Fc fusion polypeptides and thus having a nucleic acid sequence that differs from the nucleotide sequences that encode the amino acid sequences shown in Tables 1-7, or fragments thereof, are included in the invention. Moreover, nucleic acid molecules encoding ACE2-Fc fusion polypeptides from different species, such as from hominoids, and thus have a nucleotide sequence that differs from the nucleotide sequences that encode the amino acid sequences shown in Tables 1-7 are also intended to be within the scope encompassed by the present invention. For example, a chimpanzee nucleic acid sequence encoding an ACE2-Fc fusion polypeptide can be identified based on the nucleotide sequence of a human ACE2-Fc fusion polypeptide.

In one embodiment, the nucleic acid molecule(s) of the invention encodes a protein or portion thereof that includes amino acid sequences that are sufficiently homologous to amino acid sequences shown in Tables 1 and 5-7, such that the fusion protein or portion thereof is capable of binding to a coronavirus (e.g., SARS-CoV-2 and has decreased or no binding affinity for a Fc receptor (e.g., a FcγIIa receptor). Methods and assays for measuring each such biological property are well-known in the art and representative, non-limiting embodiments are described in the Examples below and Definitions above.

As used herein, the language “sufficiently homologous” refers to proteins or portions thereof which have amino acid sequences which include a minimum number of identical or equivalent (e.g., an amino acid residue that has a similar side chain as an amino acid residue in an amino acid sequence shown in Tables 1-7, or fragment thereof) amino acid residues to an amino acid sequence shown in Tables 1-7, or fragment thereof, such that the protein or portion thereof (i.e., the ACE2 extracellular domain, or fragment thereof) binds to a coronavirus (e.g., SARS-CoV-2).

In another embodiment, the protein is at least about 50%, or at least about 60%, 70%, 75%, 80%, 85%, 90%, 91%, 92%, 93%, 94%, 95%, 96%, 97%, 98%, 99% or more homologous to the entire amino acid sequence of an amino acid sequence shown in Tables 1-7, or a fragment thereof

In some embodiments, portions of proteins encoded by the ACE2-Fc fusion nucleic acid molecule encompassed by the present invention are biologically active portions of the ACE2-Fc fusion polypeptide. As used herein, the term “biologically active portion of the ACE2-Fc fusion polypeptide” is intended to include a portion, i.e., the ACE2 extracellular domain, or fragment thereof, of the ACE2-Fc fusion polypeptide that binds to coronaviruses (e.g., SARS-CoV2 virus). In some embodiments, the ACE2-Fc fusion polypeptide does not possess other activities of a full length ACE2 polypeptide.

Standard binding assays, e.g., immunoprecipitations and yeast two-hybrid assays, as described herein, or functional assays, e.g., RNAi or overexpression experiments, can be performed to determine the ability of an ACE2-Fc fusion polypeptide or a biologically active fragment thereof to maintain coronavirus binding activity of the full-length ACE2 polypeptide.

The invention further encompasses nucleic acid molecules that differ from the nucleotide sequences that encode the amino acid sequences shown in Tables 1-7, or fragment thereof due to degeneracy of the genetic code and thus encode the same polypeptides, or fragments thereof. In another embodiment, a nucleic acid molecule of the invention has a nucleotide sequence encoding a protein having an amino acid sequence shown in Tables 1-7, or fragment thereof, or a protein having an amino acid sequence which is at least about 70%, 75%, 80%, 85%, 90%, 91%, 92%, 93%, 94%, 95%, 96%, 97%, 98%, 99% or more homologous to an amino acid sequence shown in Tables 1-7, or fragment thereof, or differs by at least 1, 2, 3, 5 or 10 amino acids but not more than 30, 20, 15 amino acids from an amino acid sequence shown in Tables 1-7. In another embodiment, a nucleic acid encoding an ACE2-Fc fusion polypeptide consists of a nucleic acid sequence encoding a portion of a full-length ACE2-Fc fusion polypeptide of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

The skilled artisan will appreciate that changes can be introduced by mutation into the nucleotide sequences encoding the polypeptides having the amino acid sequences shown in Tables 1-7, or fragment thereof, thereby leading to changes in the amino acid sequence of the encoded ACE2-Fc fusion polypeptide, without altering the functional ability of the modified polypeptide. For example, nucleotide substitutions leading to amino acid substitutions at “non-essential” amino acid residues can be made in the sequence. A “non-essential” amino acid residue is a residue that can be altered from the sequence of the ACE2-Fc fusion polypeptide (e.g., the sequences shown in Tables 1-7, or fragment thereof) without significantly altering the activity of the ACE2-Fc fusion polypeptide, whereas an “essential” amino acid residue is required for the ACE2-Fc fusion polypeptide activity (i.e., binding to a coronavirus (e.g., SARS-CoV-2). Other amino acid residues, however, (e.g., those that are not conserved or only semi-conserved between mouse and human) may not be essential for activity and thus are likely to be amenable to alteration without altering the ACE2-Fc fusion polypeptide activity.

Accordingly, another aspect encompassed by the present invention pertains to nucleic acid molecules encoding ACE2-Fc fusion polypeptides that contain changes in amino acid residues that are not essential for the ACE2-Fc fusion polypeptide activity. Such ACE2-Fc fusion polypeptides differ in amino acid sequence from an amino acid sequences shown in Tables 1-7, or fragment thereof, yet retain the ACE2-Fc fusion polypeptide's coronavirus binding activity described herein. In one embodiment, the nucleic acid molecule comprises a nucleotide sequence encoding a protein, wherein the protein lacks one or more modified ACE2-Fc fusion polypeptide domains (e.g., the hinge or Fc domain may be lacking, but the ACE2 extracellular domain is necessarily present as it is responsible for binding the coronavirus). As stated in the Definitions section, the structure-function relationship of ACE2-Fc fusion polypeptide is known such that the ordinarily skilled artisan readily understands the regions that may be mutated or otherwise altered while preserving the coronavirus binding activity of the ACE2 domain.

“Sequence identity or homology”, as used herein, refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from deletions or insertions in one of the sequences are counted as mismatches.

The comparison of sequences and determination of percent homology between two sequences can be accomplished using a mathematical algorithm. The alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In an embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

A nucleic acid molecule encoding a ACE2-Fc fusion polypeptide comprising domains that are homologous to the protein domains shows in Tables 1-7, or fragment thereof, can be created by introducing one or more nucleotide substitutions, additions or deletions into the nucleotide sequences that encode the polypeptide domains, or fragments thereof, or a homologous nucleotide sequence such that one or more amino acid substitutions, additions or deletions are introduced into the encoded protein. Mutations can be introduced into a nucleotide sequence encoding the polypeptide domains shown in Tables 1-7, or fragments thereof, or the homologous nucleotide sequence by standard techniques, such as site-directed mutagenesis and PCR-mediated mutagenesis. In some embodiments, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), branched side chains (e.g., threonine, valine, isoleucine) and aromatic side chains (e.g., tyrosine, phenylalanine, tryptophan, histidine). Thus, a predicted nonessential amino acid residue in the ACE2-Fc fusion polypeptide is replaced with another amino acid residue from the same side chain family. Alternatively, in another embodiment, mutations can be introduced randomly along all or part of an ACE2-Fc fusion polypeptide coding sequence, such as by saturation mutagenesis, and the resultant mutants can be screened for the ACE2-Fc fusion polypeptide activity described herein to identify mutants that retain the ACE2-Fc fusion polypeptide activity. Following mutagenesis of a nucleotide sequence encoding one or more polypeptide domains shown in Tables 1-7, or fragments thereof, the encoded protein can be expressed recombinantly (as described herein) and the activity of the protein can be determined using, for example, assays described herein.

The levels of the ACE2-Fc fusion polypeptides may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In some embodiments, the levels of the ACE2-Fc fusion polypeptides are ascertained by measuring gene transcript (e.g., mRNA), by a measure of the quantity of translated protein, or by a measure of gene product activity. Expression levels can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In a particular embodiment, the ACE2-Fc fusion polypeptide mRNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrix™ gene chip array. A skilled artisan can readily adapt known mRNA detection methods for use in detecting the level of the ACE2-Fc fusion mRNA expression levels.

An alternative method for determining the ACE2-Fc fusion mRNA expression level in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to the ACE2-Fc fusion polypeptide mRNA.

As an alternative to making determinations based on the absolute the ACE2-Fc fusion polypeptide expression level, determinations may be based on the normalized ACE2-Fc fusion polypeptide expression level. Expression levels are normalized by correcting the absolute ACE2-Fc fusion polypeptide expression level by comparing its expression to the expression of a non-ACE2-Fc fusion polypeptide gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of an ACE2-Fc fusion polypeptide can also be detected and/or quantified by detecting or quantifying the expressed polypeptide. The ACE2-Fc fusion polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (MA), enzyme-linked immunosorbent assays (ELISAs), immunofluorescent assays, Western blotting, and the like. A skilled artisan can readily adapt known protein/antibody detection methods for use in determining whether cells express the ACE2-Fc fusion polypeptide.

b. Recombinant Expression Vectors and Host Cells

Another aspect of the invention pertains to the use of vectors, e.g., expression vectors, containing a nucleic acid encoding an ACE2-Fc fusion polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In one embodiment, adenoviral vectors comprising a ACE2-Fc fusion nucleic acid molecule are used.

The recombinant expression vectors encompassed by the present invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of the ACE2-Fc fusion polypeptide in prokaryotic or eukaryotic cells. For example, the ACE2-Fc fusion polypeptide can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the ACE2-Fc fusion polypeptide is cloned into a pGEX expression vector to create a vector encoding a fusion protein comprising, from the N-terminus to the C-terminus, GST-thrombin cleavage site-ACE2-Fc fusion polypeptide. The fusion protein can be purified by affinity chromatography using glutathione-agarose resin. Recombinant ACE2-Fc fusion polypeptide unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those utilized in, for example, E. coli (Wada et al. (1992) Nucleic Acids Res. 20:2111-2118). Such alteration of nucleic acid sequences of the invention can be carried out by standard DNA synthesis techniques.

In another embodiment, the ACE2-Fc fusion polypeptide expression vector is a yeast expression vector. Examples of vectors for expression in yeast S. cerivisae include pYepSec1 (Baldari, et al., (1987) EMBO J. 6:229-234), pMfa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, the ACE2-Fc fusion polypeptide can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO 1 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Another aspect encompassed by the present invention pertains to host cells into which a recombinant expression vector or nucleic acid encompassed by the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the ACE2-Fc fusion polypeptide can be expressed in bacterial cells such as E. coli, insect cells, yeast or mammalian cells (such as Fao hepatoma cells, primary hepatocytes, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A cell culture includes host cells, media and other byproducts. Suitable media for cell culture are well known in the art. A ACE2-Fc fusion polypeptide or fragment thereof, may be secreted and isolated from a mixture of cells and medium containing the polypeptide. Alternatively, an ACE2-Fc fusion polypeptide or fragment thereof, may be retained cytoplasmically and the cells harvested, lysed and the fusion protein isolated. An ACE2-Fc fusion polypeptide or fragment thereof, may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the ACE2-Fc fusion polypeptide or a fragment thereof.

In some embodiments, the ACE2-Fc fusion polypeptide, or biologically active fragment thereof, may be fused to a heterologous polypeptide. In certain embodiments, the fused polypeptide has greater half-life than the corresponding unfused ACE2 polypeptide. In other embodiments, heterologous tags can be used for purification purposes (e.g., epitope tags) according to standards methods known in the art.

Thus, a nucleotide sequence encoding all or a selected portion of the ACE2-Fc fusion polypeptide may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant ACE2-Fc fusion polypeptides, or fragments thereof, by microbial means or tissue-culture technology in accord with the subject invention.

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs.

In certain embodiments, the ACE2-Fc fusion polypeptide, or fragment thereof, may be synthesized chemically, ribosomally in a cell free system, or ribosomally within a cell. Chemical synthesis may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., (1993) Curr. Opin. Biotech., vol. 4, p 420; M. Miller, et al., (1989) Science: vol. 246, p 1149; A. Wlodawer, et al., (1989) Science: vol. 245, p 616; L. H. Huang, et al., (1991) Biochemistry: vol. 30, p 7402; M. Sclmolzer, et al., (1992) Int. J. Pept. Prot. Res.: vol. 40, p 180-193; K. Rajarathnam, et al., (1994) Science: vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., (1992) J. Biol. Chem.: vol. 267, p 3852; L. Abrahmsen, et al., (1991) Biochemistry: vol. 30, p 4151; T. K. Chang, et al., (1994) Proc. Natl. Acad. Sci. USA 91: 12544-12548; M. Schnlzer, et al., (1992) Science: vol., 3256, p 221; and K. Akaji, et al., (1985) Chem. Pharm. Bull. (Tokyo) 33: 184).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Selectable markers include, but are not limited to, those which confer resistance to drugs, such as G418, hygromycin and methotrexate. Nucleic acid encoding a selectable marker can be introduced into a host cell on the same vector as that encoding the ACE2-Fc fusion polypeptide or can be introduced on a separate vector. Cells stably transfected with the introduced nucleic acid can be identified by drug selection (e.g., cells that have incorporated the selectable marker gene will survive, while the other cells die).

A host cell encompassed by the present invention, such as a prokaryotic or eukaryotic host cell in culture, can be used to produce (i.e., express) the ACE2-Fc fusion polypeptide. Accordingly, the invention further provides methods for producing the ACE2-Fc fusion polypeptide using the host cells of the invention. In one embodiment, the method comprises culturing the host cell of invention (into which a recombinant expression vector encoding the ACE2-Fc fusion polypeptide has been introduced) in a suitable medium until the ACE2-Fc fusion polypeptide is produced. In another embodiment, the method further comprises isolating the ACE2-Fc fusion polypeptide from the medium or the host cell.

c. ACE2-Fc Fusion Polypeptides

The present invention also provides soluble, purified and/or isolated forms of ACE2-Fc fusion polypeptides that bind coronaviruses (e.g., SARS-CoV-2, the causative agent of COVID-19, and SARS-CoV-1, the causative agent of SARS) and competitively inhibits the virus from binding to ACE2 endogenously expressed on a cell surface. The ACE2-Fc fusion protein can comprise the extracellular domain, or a portion thereof, of an ACE2 polypeptide and the Fc domain, or a fragment thereof, of an immunoglobulin polypeptide (e.g., an IgG1, IgG2, or IgG4 polypeptide). The ACE2 extracellular domain, or fragment thereof, and the Fc domain are separated by a hinge polypeptide. In some embodiments, the ACE2 extracellular domain, or fragment thereof, and the Fc domain are separated by a single proline or a cysteine-proline dipeptide. In some embodiments, the nucleic acid that encodes an ACE2-Fc fusion polypeptide comprises an amino acid sequence having at least 70%, 80%, 90% or greater identity to the human ACE2 extracellular domain, or fragment thereof, amino acid sequence of any one of SEQ ID NOs: 1-4, an amino acid sequence of a hinge that comprises an amino acid sequence of any one of SEQ ID NOs: 5-32 or 52, and an amino acid sequence having at least 70%, 80%, 90% or greater identity to an Fc domain, or fragment thereof, amino acid sequence of any one of SEQ ID NOs: 33-42 or 55. The ACE2-Fc fusion polypeptide can be for use according to methods described herein.

In one aspect, an ACE2-Fc fusion polypeptide comprises a human ACE2 extracellular domain, or fragment thereof, having an amino acid sequence of any one of SEQ ID NOs: 1-4, which comprise amino acid substitutions analogous to H374N and H378N amino acid substitutions in a wild-type ACE2 polypeptide or a human ACE2 extracellular domain, or fragment thereof, having an amino acid sequence of any one of SEQ ID NOs: 1-4, which comprise H374N and H378N amino acid substitutions with 1 to about 20 additional conservative amino acid substitutions. In some embodiments, the ACE2 extracellular domain comprises amino acid substitutions at positions corresponding to H374N and H378N. In some embodiments, the ACE2 extracellular domain comprises one or more amino acid substitutions at positions corresponding to R273Q, H345A, H345L, H505A, and H505L. In some embodiments, the ACE2 extracellular domain comprises amino acid substitutions at positions corresponding to R169Q, W271Q, and K481Q. The above amino acid substitutions are relative to the full length ACE2 polypeptide. In some embodiments, the nucleic acid encodes an ACE2 extracellular domain comprising amino acids 18-600, 18-615, 18-708, or 18-740 of the full length ACE2 polypeptide.

Point substitutions that reduce the enzymatic activity of the ACE2 extracellular domain are contemplated herein. In some embodiments, point substitutions result in a greater reduction of ACE2 activity, while others decreased the binding avidity of ACE2 for the SARS-CoV-2 or SARS-CoV spike proteins (S-proteins). Retention of avid binding to the S-proteins is needed.

To be clear, SEQ ID NOs: 1-4 comprise asparagine residues at amino acid residue positions 357 and 361. The residues decrease or eliminate the enzymatic activity of an ACE2 polypeptide without inhibiting binding to a coronavirus. The amino acid sequence of any ACE2 extracellular domain, or fragment thereof, polypeptide described herein can also be at least 50, 55, 60, 65, 70, 75, 80, 85, 90, 91, 92, 93, 94, 95, 96, 97, 98, 99, or 99.5% identical to a human ACE2 extracellular domain, or fragment thereof, amino acid sequence, or a fragment thereof.

The ACE2-Fc fusion polypeptide encompassed by the present invention also comprises a Fc domain fragment. The Fc domain of an intact IgG antibody, by binding to its Fcγ receptor (e.g., a FcγIIa receptor), is responsible for antibody effector functions such as antibody-dependent cellular cytotoxicity and antibody-dependent cellular phagocytosis (Kand and Jung (2019) Exp. & Mol. Med., 51: 138-46; Moi et al. (2010) 1 Gen. Virol., 91: 103-11; Jiang et al. (2011) Nat. Reviews Drug Discovery, 10: 101-10). Recombinant Fc domains can be engineered such that the Fc domain does not bind to a Fcγ receptor (e.g., a FcγIIa receptor), or at least has attenuated Fcγ receptor (e.g., a FcγIIa receptor) binding activity (Saunders (2019) Frontiers Immunology, 10:Art. 1296; Shotlothauer et al. (2016) Prot. Eng., Design & Selection, 29(10):457-66). Additionally, the Fc domain, or fragment thereof, provides additional benefits, such as, but not limited to, increased life span in circulation (Saunders (2019)) when incorporated into a therapeutic polypeptide (i.e., an ACE2-Fc fusion polypeptide).

In some embodiments, the Fc domain has an amino acid sequence of any one of SEQ ID NOs: 33-42 or 55. In some embodiments, the Fc domain fragment is derived from an IgG1, IgG2, or IgG4 Fc domain. In some embodiments, the Fc domain fragment has reduced binding affinity for a Fcγ receptor (e.g., a FcγIIa receptor). Amino acid substitutions in the Fc domain can reduce the binding affinity for a Fcγ receptor (e.g., an FcγIIa receptor); therefore, in some embodiments, an ACE2-Fc fusion polypeptide encompassed by the present invention comprises a Fc domain fragment derived from an IgG1 Fc domain that has an L234A, L235A, L235E, N297A, N297D, P329G, or a combination thereof, amino acid substitution. In some embodiments, an ACE2-Fc fusion polypeptide encompassed by the present invention comprises an Fc domain fragment derived from an IgG1 Fc domain that has L234A, L235A, and P329G amino acid substitutions. In one embodiment, the Fc domain fragment is derived from an IgG4 Fc domain and has L235E and P329G amino acid substitutions. In one embodiment, the Fc domain fragment is derived from an IgG1 Fc domain and has a P329G amino acid substitution. In one embodiment, the Fc domain fragment is derived from an IgG4 Fc domain and has a L235E amino acid substitution. In some embodiments, the Fc domain fragment is derived from an IgG1 Fc domain and has L234A and L235A amino acid substitutions. In one embodiment, the Fc domain fragment is derived from a wild-type IgG4 Fc domain. In one embodiment, the Fc domain fragment is derived from an IgG1 Fc domain and has a N297D amino acid substitutions. In one embodiment, the Fc domain fragment is derived from an IgG1 Fc domain and has a N297A amino acid substitution. In some embodiments, the Fc domain fragment is derived from an IgG1 Fc domain and has a N297Q amino acid substitution. In one embodiment, the Fc domain fragment is derived from a wild type IgG2 Fc domain. The amino acid residue numbering throughout is relative to a full length protein or domain (i.e., a full length ACE2 polypeptide or full length IgG Fc domain). The amino acid sequence of any Fc domain fragment polypeptide described herein can also be at least 50, 55, 60, 65, 70, 75, 80, 85, 90, 91, 92, 93, 94, 95, 96, 97, 98, 99, or 99.5% identical to a human Fc domain amino acid sequence, or a fragment thereof.

The ACE2 extracellular domain and Fc domain fragments of the ACE2-Fc fusion polypeptide encompassed by the present invention can be fused to an additional polypeptide that separates the extracellular and Fc domains. The terms “hinge” or “hinge region” or “hinge polypeptide” are used interchangeably to refer to such an intervening polypeptide. In some embodiments, the hinge region that corresponds with the isotype of the Fc domain that is used. For example, if an IgG4 hinge sequence is used, an S228P substitution can minimize Fab-arm exchange. In some embodiments, the hinge region can comprise an amino acid sequence of any SEQ ID NO in tables 2-4. In some embodiments, the hinge region will comprise an amino acid residue that is analogous to an amino acid substitution in a Fc domain that reduces binding affinity of the Fc fragment for a Fcγ receptor (e.g., a FcγIIa receptor). For example, in some embodiments, the ACE2-Fc fusion polypeptide comprises a hinge region that comprises an amino acid that is analogous to the S228P amino acid substitution in the IgG4 Fc domain that is associated with reduced Fab-arm exchange. In some embodiments, the ACE2-Fc fusion polypeptide encompassed by the present invention comprises a hinge region comprising the S228P amino acid substation and a Fc fragment comprising the L235E amino acid substitution, and the polypeptide is said to have an IgG4-SPLE Fc domain. In some embodiments, the L235E substitution confers reduced affinity of an IgG4 Fc domain for Fcγ receptors (e.g., a FcγIIa receptor).

In one embodiment, the ACE2-Fc fusion polypeptide comprises an ACE2 extracellular domain having the amino acid sequence of SEQ ID NO 1, which includes the H374N and H378N substitutions; a hinge region comprising the amino acid sequence of SEQ ID NO: 10; and a Fc domain fragment having the amino acid sequence of SEQ ID NO: 33. In another embodiment, the ACE2-Fc fusion polypeptide comprises an ACE2 extracellular domain having the amino acid sequence of SEQ ID NO: 4, which includes the H374N and H378N substitutions; a hinge region comprising the amino acid sequence of SEQ ID NO: 6; and a Fc domain fragment having the amino acid sequence of SEQ ID NO: 33. In some embodiments, the ACE2-Fc fusion polypeptide comprises the amino acid sequence of SEQ ID NO: 48, 49, 56, or 57. In some embodiments, the ACE2-Fc fusion polypeptide comprises an amino acid sequence having at least 70%, 80%, or 90% or greater identity to the amino acid sequence of SEQ ID NO: 48, 49, 56, or 57. In some embodiments, the ACE2-Fc fusion polypeptide encompassed by the present invention is encoded by a nucleic acid molecule comprising a nucleotide sequence having at least 70%, 80%, or 90% or greater identity to the nucleotide sequence of SEQ ID NO: 50, 51, 58, or 59.

Any ACE2-Fc fusion polypeptide, or fragment thereof, described herein has binding affinity for a coronavirus (e.g., SARS-CoV-1, SARS-CoV-2, etc.), decreased or eliminated ACE2 enzymatic activity, and decreased or eliminated binding of the Fc domain, or fragment thereof, to a Fc receptor (e.g., an FcγIIa receptor). Although monoclonal antibodies can sometimes exhibit cross-reactivity between related viruses, they typically exhibit higher avidity for the virus against which they were raised and are often only partially neutralizing against novel related virus unless re-engineered and optimized. By contrast, the ACE2-Fc fusion polypeptides encompassed by the present invention is an optimized soluble form of the actual receptor to which ACE2-dependent coronaviruses bind and therefore should function as a competitive inhibitor for any future novel coronaviruses that exploit ACE2 as a cell-surface receptor.

In certain embodiments, the decreased or eliminated ACE2 enzymatic activity and the decreased binding of the Fc domain, or fragment thereof, to a Fc receptor are decreased by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 76-fold, at least 77-fold, at least 78-fold, at least 79-fold, at least 80-fold, at least 81-fold, at least 82-fold, at least 83-fold, at least 84-fold, at least 85-fold, at least 86-fold, at least 87-fold, at least 88-fold, at least 89-fold, at least 90-fold, at least 91-fold, at least 92-fold, at least 93-fold, at least 94-fold, at least 95-fold, at least 96-fold, at least 97-fold, at least 98-fold, at least 99-fold, at least 100-fold, or any range inclusive, such as 5-fold to 20-fold relative to a full length ACE2 polypeptide or a wild-type Fc domain, or fragment thereof.

In another aspect, the present invention contemplates a composition comprising an ACE2-Fc fusion polypeptide described herein and less than about 25%, or alternatively 15%, or alternatively 5%, contaminating biological macromolecules or polypeptides.

The present invention further provides compositions related to producing, detecting, or characterizing a ACE2-Fc fusion polypeptide, or fragment thereof, such as nucleic acids, vectors, host cells, and the like. Such compositions may serve as compounds that modulate a ACE2-Fc fusion polypeptide's expression and/or activity.

The ACE2-Fc fusion polypeptide of the invention is a fusion protein containing an Fc domain, or fragment thereof, which increases its solubility and bioavailability and/or facilitates its purification, identification, detection, and/or structural characterization. In some embodiments, it may be useful to express an ACE2-Fc fusion polypeptide in which the fusion partner enhances fusion protein stability in blood plasma and/or enhances systemic bioavailability. Exemplary additional domains that can be incorporated into an ACE2-Fc fusion polypeptide encompassed by the present invention, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type 21 secretion system-targeting peptides, transcytosis domains, nuclear localization signals, etc. In various embodiments, an ACE2-Fc fusion polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases.

In still another embodiment, an ACE2-Fc fusion polypeptide may be labeled with a fluorescent label to facilitate their detection, purification, or structural characterization. In an exemplary embodiment, an ACE2-Fc fusion polypeptide of the invention may be fused to a heterologous polypeptide sequence which produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

In some embodiments, the ACE2-Fc fusion polypeptide or portion thereof comprises an amino acid sequence that is sufficiently homologous to an amino acid sequence shown in Tables 1, 5, or 7, such that the ACE2-Fc fusion polypeptide or portion thereof is enzymatically dead or has decreased enzymatic activity but can bind to a coronavirus and a Fc fragment that is not recognized by a Fcγ receptor (e.g., a FcγIIa receptor). In one embodiment, the ACE2-Fc fusion polypeptide or portion thereof comprises an amino acid sequence that is sufficiently homologous to an amino acid sequence shown in Table 1, such that the ACE2-Fc fusion polypeptide or portion thereof is enzymatically dead or has decreased enzymatic activity but can bind to a coronavirus. In one embodiment, the ACE2-Fc fusion polypeptide or portion thereof comprises an amino acid sequence that is sufficiently homologous to an amino acid sequence shown in Table 5, such that the ACE2-Fc fusion polypeptide or portion thereof is has increased stability relative to a wild type sequence for a Fc polypeptide and/or does not bind to a Fcγ receptor (e.g., a FcγIIa receptor). In an embodiment, the ACE2-Fc fusion polypeptides comprises an amino acid sequence shown in Table 1, or fragment thereof, each of which comprises amino acid substitutions analogous to H374N and H378N amino acid substitutions in a wild-type ACE2 polypeptide, or an amino acid sequence which is at least about 50%, 55%, 60%, 65%, 70%, 75%, 80%, 85%, 90%, 91%, 92%, 93%, 94%, 95%, 96%, 97%, 98%, 99% or more homologous to the amino acid sequence shown in Table 1, or fragment thereof, each of which comprises H374N and H378N amino acid substitutions. To be clear, the amino acid sequences shown in table 1 comprise asparganine residues at amino acid residue positions 357 and 361. These residues decrease or eliminate ACE2 enzymatic activity but do not inhibit ACE2 binding of a coronavirus. The ACE2-Fc fusion polypeptides also comprises an Fc domain, or fragment thereof, polypeptide having an amino acid sequence shown in Table 5, or fragment thereof, or an amino acid sequence which is at least about 50%, 55%, 60%, 65%, 70%, 75%, 80%, 85%, 90%, 91%, 92%, 93%, 94%, 95%, 96%, 97%, 98%, 99% or more homologous to the amino acid sequence shown in Table 1, or fragment thereof. The ACE2 domain and the Fc domain, or fragment thereof, of the ACE2-Fc fusion polypeptide may be separated by a hinge region. In some embodiments, the hinge region has an amino acid sequence shown in Tables 2, 3, or 4. In some embodiments, the Fc domain, or fragment thereof, polypeptide will comprise at least one amino acid substitution that attenuates the fragment's ability to bind to an Fc receptor (e.g., a FcγIIa receptor). In some embodiments, the hinge region will comprise at least one amino acid substitution that attenuates the fragment's ability to bind to an Fc receptor (e.g., a FcγIIa receptor).

The ACE2-Fc fusion polypeptide encompassed by the present invention can be designed to reduce or eliminate antibody dependent enhancement (ADE) of a coronavirus infection. For example, a Fc domain, or fragment thereof, binds to the ACE2-Fc fusion protein, the amino acid sequence of the Fc domain, or fragment thereof, can be optimized to reduce binding of the fragment to a Fc receptor (e.g., a FcγIIa receptor). The hinge region can comprise an amino acid sequence that attenuates the ability of the Fc domain, or fragment thereof, to bind to an Fc receptor. For example, the ACE2-Fc fusion polypeptide may comprise an amino acid substitution in its hinge (e.g., S228P) that reduces Fab-arm exchange. In some embodiments, the ACE2-Fc receptor comprises an amino acid substitution in the hinge region or in the Fc domain, or fragment thereof, (e.g., L235E), which reduce or eliminate the ability of the Fc domain, or fragment thereof, to bind to an Fc receptor.

The ACE2-Fc fusion polypeptides of the precent invention can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An ACE2-Fc fusion polypeptide-encoding nucleic acid can be cloned into such an expression vector such that the fusion moieties are linked in-frame.

The present invention also pertains to fusion proteins comprising amino acid sequence homologues of the ACE2 extracellular domain and the Fc domain. Homologues of the human ACE2 extracellular domain and the Fc domain can be generated by mutagenesis, e.g., discrete point mutation or truncation of the human ACE2 extracellular domain and the Fc domain, respectively. As used herein, the term “homologue” refers to a variant form of the human ACE2 extracellular domain and/or the Fc domain polypeptide. In one embodiment, treatment of a subject with a homologue having a subset of the biological activities of the naturally occurring form of the protein has fewer side effects in a subject relative to treatment with the naturally occurring form of the ACE2 extracellular domain and/or the Fc domain, or a fragment thereof.

In an alternative embodiment, homologues of the human ACE2 extracellular domain or the Fc domain, or a fragment thereof, can be identified by screening combinatorial libraries of mutants, e.g., truncation mutants, of the ACE2 extracellular domain or the Fc domain, or a fragment thereof. In one embodiment, a variegated library of the ACE2 extracellular domain or the Fc domain variants is generated by combinatorial mutagenesis at the nucleic acid level and is encoded by a variegated gene library. A variegated library of the modified ACE2 extracellular domain or the Fc domain variants can be produced by, for example, enzymatically ligating a mixture of synthetic oligonucleotides into gene sequences such that a degenerate set of potential ACE2 extracellular domain or the Fc domain sequences is expressible as individual polypeptides, or alternatively, as a set of larger fusion proteins (e.g., for phage display) containing the set of the ACE2 extracellular domain or the Fc domain, or a fragment thereof, sequences therein. There are a variety of methods which can be used to produce libraries of potential ACE2 extracellular domain or the Fc domain, or a fragment thereof, homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ACE2 extracellular domain or the Fc domain, or a fragment thereof, sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

### III. Pharmaceutical Compositions

In another aspect, the present invention provides pharmaceutically acceptable compositions which comprise an ACE2-Fc fusion polypeptide comprising an amino acid sequence that has at least 70%, 80%, 90%, 95% or greater identity to an amino acid sequence shown in Table 1 and an amino acid sequence that has at least 70%, 80%, 90%, 95% or greater identity to an amino acid sequence shown in Table 5, formulated together with one or more pharmaceutically acceptable carriers (additives) and/or diluents. In an embodiment, the pharmaceutically acceptoable composition comprises an ACE2-Fc fusion polypeptide comprising an amino acid sequence that has at least 70%, 80%, 90%, 95% or greater identity to an amino acid sequence shown in Table 7

As described in detail below, the pharmaceutical compositions encompassed by the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The phrase “therapeutically-effective amount” as used herein means that amount of the ACE2-Fc fusion polypeptide that is effective for producing a therapeutic effect, e.g., a decrease or mitigation of a symptom of coronavirus infection (e.g., fever, cough, sore throat, fatigue, shortness of breath, dyspnea, tachypnea, hypoxia, etc.) at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods encompassed by the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, such as from about 5 percent to about 70 percent, or from about 10 percent to about 30 percent.

Methods of preparing these formulations or compositions include the step of bringing into association an ACE2-Fc fusion polypeptide encompassed by the present invention, with the carrier and, optionally, one or more accessory ingredients. In general, the formulations are prepared by uniformly and intimately bringing into association a respiration uncoupling agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical, nebulization, or transdermal administration of an ACE2-Fc fusion polypeptide encompassed by the present invention include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays can contain, in addition to an ACE2-Fc fusion polypeptide, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The an ACE2-Fc fusion polypeptide, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a respiration uncoupling agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more respiration uncoupling agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of a an ACE2-Fc fusion polypeptide, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the respiration uncoupling agents encompassed by the present invention are administered as pharmaceuticals, to humans and animals, they can be given per se or as a pharmaceutical composition containing, for example, 0.1 to 99.5% (such as, 0.5 to 90%) of active ingredient in combination with a pharmaceutically acceptable carrier.

Actual dosage levels of the active ingredients in the pharmaceutical compositions of this invention may be determined by the methods encompassed by the present invention so as to obtain an amount of the active ingredient, which is effective to achieve the desired therapeutic response for a particular subject, composition, and mode of administration, without being toxic to the subject.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

This invention is further illustrated by the following examples which should not be construed as limiting. The contents of all references, patents and published patent applications cited throughout this application, as well as the Figures, are incorporated herein by reference.

### IV. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including cell and non-cell based assays. In one embodiment, the assays provide a method for identifying whether a coronavirus is likely to respond to treatment with an ACE2-Fc fusion polypeptide alone or in combination with other viral therapies, such as in a human, by using a non-cell assay to determine the binding affinity of the ACE2-Fc fusion polypeptide for a coronavirus or a cell culture assay to determine the prohibitive effect the ACE2-Fc fusion polypeptide has on infection and/or ADE.

For example, in a direct binding assay, an ACE2 fusion polypeptide (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of an ACE2-Fc to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between a coronavirus and a full length ACE2 polypeptide can be accomplished by determining the ability of a ACE2-fusion polypeptide encompassed by the present invention to modulate the infectivity of a coronavirus.

The present invention further pertains to ACE2-Fc identified by the above-described screening assays. Accordingly, it is within the scope of this invention to further use an ACE2-Fc fusion polypeptide identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a coronavirus in the context of a biological sample (e.g., blood, serum, cells, or tissue) to thereby determine whether an individual infected with the virus is likely to respond to treatment with an ACE2-Fc fusion polypeptide encompassed by the present invention. Such assays can be used for prognostic or predictive purpose alone, or can be coupled with a therapeutic intervention to thereby prophylactically treat an individual prior to the onset or after the onset of symptoms associated with a coronavirus infection. The skilled artisan will appreciate that any method can use one or more (e.g., combinations) of the ACE2-Fc fusion polypeptides described herein, such as those in the tables, figures, examples, and otherwise described in the specification.

Another aspect of the present invention pertains to monitoring the influence of agents (i.e., the ACE2-Fc fusion polypeptide encompassed by the present invention) on the expression or activity of a biomarker described herein. These and other agents are described in further detail in the following sections.

The skilled artisan will also appreciate that, in certain embodiments, the methods of the present invention implement a computer program and computer system. For example, a computer program can be used to perform the algorithms described herein. A computer system can also store and manipulate data generated by the methods encompassed by the present invention, which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-infected tissue of a subject and/or profiles generated from population-based distributions of informative loci of interest in relevant populations of the same species can be stored and later compared to that of a sample derived from the infected tissue of the subject or tissue suspected of being infected of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is infected with a coronavirus that is likely to respond to treatment with one or more of the ACE2-Fc fusion polypeptides encompassed by the present invention. In some embodiments, the present invention is useful for classifying a sample (e.g., from a subject) as associated with or at risk of a coronavirus infection that will or will not respond to treatment with an ACE2-Fc fusion polypeptide encompassed by the present invention using a statistical algorithm and/or empirical data (e.g., the amount or activity of a biomarker described herein, such as in the tables, figures, examples, and otherwise described in the specification).

An exemplary method for detecting the amount or activity of a coronavirus described herein, and thus useful for classifying whether a sample is likely or unlikely to respond to treatment with an ACE2-Fc fusion polypeptide encompassed by the present invention involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely immunotherapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of an ACE2-Fc fusion polypeptide based upon the diagnosis.

In one embodiment, the methods further involve obtaining a control biological sample (e.g., biological sample from a subject who does not have a coronavirus infection or whose infection is susceptible to viral sequestration by an ACE2-Fc fusion polypeptide, a biological sample from the subject during before treatment, or a biological sample from the subject during or after treatment.

d. Prognostic Assays

The diagnostic methods described herein can furthermore be utilized to identify subjects having or at risk of a coronavirus infection that is likely or unlikely to be responsive to ACE2-Fc fusion polypeptides. The assays described herein, such as the preceding diagnostic assays or the following assays, can be utilized to identify a subject having or at risk of being infected with a coronavirus. Alternatively, the prognostic assays can be utilized to identify a subject having or at risk for developing a disorder associated with a coronavirus infection, such as pneumonia. Furthermore, the prognostic assays described herein can be used to determine whether a subject can be administered an ACE2-Fc fusion polypeptide to treat a disease or disorder associated with an ACE2-Fc fusion polypeptide encompassed by the present invention.

e. Treatment Methods

The present invention provides for both prophylactic and therapeutic methods of treating a subject at risk of (or susceptible to) a coronavirus infection, including SARS-CoV-2 infection, which is the causative agent of COVID-19. In some embodiments, an ACE2-Fc fusion protein encompassed by the present invention is administered to a subject infected or at risk of becoming infected with a coronavirus. The administered ACE2-Fc fusion protein is a pharmacologically optimized soluble form of ACE2, the cell-surface receptor for the SARS-CoV-2 coronavirus, and functions as a competitive inhibitor of viral infection by binding to the receptor binding domain (RBD) of the SARS-CoV-2 spike protein, preventing its attachment to cell-surface ACE2. The ACE2-Fc fusion protein comprises an Fc fragment that interrupts antibody dependent enhancement (ADE), which may cause or worsen the severity of acute respiratory distress syndrome (ARDS) in COVID-19 patients.

A drawback of the therapeutic use of enzymatically active ACE2 for SARS-CoV-2, whether or not it is in an Fc fusion format, is the potential for hemodynamic side effects and electrolyte abnormalities. Wild type ACE2 is an ectoenzyme that degrades angiotensin II, a component of the Renin-Angiotensin-Aldosterone System (RAAS) pathway and is, therefore, a natural RAAS inhibitor which regulates blood pressure, fluid, and electrolyte balance (primarily sodium) in the body. Therapeutics that utilize wild type ACE2 would be expected to cause side effects that mirror the physiologic effects of RAAS inhibition, particularly at higher doses or in patients who are either acutely ill, in a state of relative volume depletion, or have chronic conditions that result in RAAS dependency such as renovascular stenosis or heart failure. Side effects from RAAS inhibition can include hypotension, hypovolemia, kidney injury, decrease blood pressure and potentially result in hyponatremia or electrolyte changes. Thus, ACE2 treatment of COVID-19 patients, a population which can be critically ill, may result in dose-limiting side effects, which raises concerns for the use of active ACE2 in both therapeutic and prophylactic settings.

Enzymatically active recombinant ACE2 extracellular domain (not in an Fc fusion format) has been developed for the treatment of hypertension due to pathologic RAAS activation. In a phase-1 randomized dose escalation study of healthy volunteers, it did not have an effect on blood pressure or heart rate and was well tolerated at the doses studied (Haschke et al. (2013) Clin Pharmacokinet., 52(9):783-92). But its physiologic effects in COVID-19 patients, many of whom are critically ill, may not mirror its effects in healthy volunteers. Moreover, the doses required for competitive inhibition of virus would likely be higher than those studied in the aforementioned phase-1 trial for RAAS-mediated hypertension, thus raising the concern that even non-critically ill COVID-19 patients may experience adverse hemodynamic effects and hyponatremia.

The side effects of an enzymatically active ACE2 drug would be most apparent when the RAAS system is active. This can occur in two settings: 1) states of intravascular volume depletion, such as dehydration or sepsis; or 2) chronic states of RAAS activation such as renal artery stenosis, heart failure, or cirrhosis (liver failure) (Atlas, S. A., (2007) J. Manag. Care Pharm., 13(8 Suppl B):9-20; Hricik et al. (1983) N. Engl. J. Med., 308(7):373-6; Brown et al. (1998) Circulation, 97(14):1411-20; Arroyo et al. (2011) Nat Rev Nephrol., 7(9):517-261). These chronic conditions are common in the older population, including veterans, who are more likely to develop severe COVID-19 (Krishnamurthi et al. (2018) PLoS One, 13(3):e0193996; Eibner et al. (2016) Rand Health Q., 5(4):13. Since these comorbid conditions are often clinically silent, it may not be apparent that an individual has one of these predisposing conditions before being exposed to active ACE2, and the drug may precipitate low blood pressure or dangerous electrolyte abnormalities such as hyperkalemia. These concerns are therefore relevant to the use of active ACE2 in both the therapeutic and prophylactic settings.

Indeed, enzymatically active recombinant ACE2 extracellular domain (not in an Fc fusion format) has been developed as a treatment for hypertension due to pathologic over-activation of RAAS (Haschke et al. (2013) Clin Pharmacokinet., 52(9):783-92. But as previously noted, the physiologic effects of RAAS inhibition would be most apparent when the RAAS system is active, not in healthy volunteers in a controlled setting. Moreover, the doses required for competitive inhibition of virus would likely be higher than those studied in this trial. Therefore, this trial does not assuage concerns about the safety of enzymatically active ACE2 for COVID-19 treatment or prophylaxis.

The prolonged half-life imparted by the IgG Fc domain allows for subcutaneous dosing. Several hundred milligrams can be administered with each injection, sufficient dosing for SARS-CoV-2 inhibition. The time to peak concentration is longer for biologics that are dosed subcutaneously when compared with intravenous dosing, but this is commonly accounted for by administering a larger initial loading dose followed by smaller maintenance doses. If repeat dosing is necessary, it is typically performed at the half-life, so injection would be required no more frequently than every three days. With respect to product deployment, FDA-approved Fc fusion proteins for subcutaneous injection are often available in pre-filled syringes ready for injection. Pre-filled syringes for subcutaneous injection of protein drugs are typically stable for 12-24 months with refrigeration and do not require refrigeration for transportation.

The present invention comprises an enzymatically inactive ACE2 extracellular domain. The inclusion of two point mutations in ACE2 (H374N & H378N) is known to markedly reduce its enzymatic activity (Imai et al. (2005) Nature, 436(7047):112-6). Crucially, these point mutations do not affect its binding to the SARS-CoV or SARS-CoV-2 spike proteins. ACE2-NN-Fc fusion proteins that include these point mutations still potently inhibit infection of target cells by SARS-CoV and SARS-CoV-2 pseudoviruses (Imai et al., Lei et al. (2020) Nat. Commun. 11(1):2070). The IC50 for iACE2-Fc against SARS-CoV-2 was less than 0.1 μg/mL, or less than 0.5 nanomolar.

There are several advantages associated with treating or preventing a coronavirus infection by administering an ACE2-Fc fusion encompassed by the present invention. The ACE2-Fc fusion polypeptide encompassed by the present invention does not elicit these side effects because it employs an enzymatically inactive ACE2 domain, which is not a RAAS inhibitor. Therefore, the ACE2-Fc fusion polypeptide is safe to administer to both acutely ill patients in the hospital and to individuals with chronic conditions in an unmonitored community setting. This enables its use as a prophylactic agent in addition to a therapeutic agent. Because the risk of side effects is low, use of enzymatically inactive ACE2 allows higher peak serum concentrations to be tolerable. Higher doses of the ACE2-Fc fusion polypeptide improve competitive inhibition of coronavirus (e.g., SARS-CoV-2) infection and competitive inhibition of ACE2 while also allowing for less frequent dosing (i.e., increasing the interval between dosing), thus improving the practicality of deployment in non-hospital settings.

Additionally, of the seven coronaviruses known to infect humans 0C43, HKU1, 229E, NL63, MERS-CoV, SARS-CoV, and SARS-CoV-2), three are known to utilize ACE2 (SARS-CoV, SARS-CoV-2, and NL63), including two viruses of global importance: SARS-CoV, the virus responsible for the 2004 Severe Acute Respiratory Syndrome outbreak, and SARS-CoV-2, the virus responsible for the COVID-19 pandemic (Li et al. (2003) Nature, 426(6965):450-54; Hoffman et al. (2020) Cell, 181(2):271-80 e8. Thus, because ACE2 the primary surface receptor through which SARS-CoV-2 infects cells, the ACE2-Fc fusion protein encompassed by the present invention will bind any coronavirus that utilizes ACE2 as a receptor, including future novel coronaviruses. In contrast, neutralizing monoclonal antibodies as well as vaccines against SARS-CoV-2 may not be effective against future novel coronaviruses or mutant variants of SARS-CoV-2 that arise in the population, especially if widespread treatment and vaccination exerts evolutionary pressure on the virus. This is because therapeutic neutralizing antibodies and antibodies induced by vaccination bind a defined epitope on the spike protein receptor binding domain (RBD), which may mutate to evade antibody binding while preserving ACE2 binding. Since the ACE2-Fc fusion protein to be administered to a subject in need thereof is a pharmacologically optimized form of ACE2 itself, a virus with a mutated RBD that exhibits diminished binding to the ACE2-Fc fusion protein would also exhibit diminished binding to target cells and reduced infectivity.

It is important to emphasize that the inert ACE2 extracellular domain of the ACE2-Fc fusion protein does not interrupt or affect the activity of endogenous ACE2 within the lung. Soluble ACE2-Fc fusion proteins do not affect the severity of ARDS, which ACE2 may protect against (Imai et al. (2005)).

The fusion proteins encompassed by the present invention avoid complications associated with treatments comprising administering neutralizing antibodies to a subject in need thereof. For example, antibody-dependent enhancement (ADE) is a phenomenon whereby neutralizing antibodies paradoxically worsen infection and inflammation in a viral syndrome. ADE is best characterized in dengue fever, where endogenous neutralizing antibodies raised in a first infection worsen the viral syndrome in a second infection, typically when the second infection is with a different serotype of the virus against which the antibodies are partially neutralizing (Tirado et al. (2003) Viral Immunol., 16(1):69-86; Takada et al. (2003) Rev Med Virol., 13(6):387-98; Khandia et al. (2018) Front Immunol., 9:597. This phenomenon is Fcγ receptor-dependent. Antibodies first bind the virus and then bind Fcγ receptors on immune cells, mediating viral entry and activating the immune cells (Wan et al. (2020) 1 Virol., 94(5). This results in both viral replication in the immune cell and release of inflammatory cytokines such as TNF-α and IL-6 (Boonnak et al. (2008) J. Virol., 82(8):3939-51. These cytokines are also associated with tissue damage, shock, lung pathology, and ARDS.

There is evidence for ADE in HIV and Ebola virus, as well as in coronaviruses (Wan et al. (2020); Beck et al. (2008) Vaccine, 26(24):3078-85; Takada et al. (2001) J. Virol., 75(5):2324-30; Takada et al. (2003) J. Virol., 77(13):7539-44; Corapi et al. (1992) J. Virol., 66(11):6695-705; Hohdatsu et al. (1998) J. Vet. Med. Sci., 60(1):49-55; Vennema et al. (1990) J. Virol., 64(3):1407-9; Wang et al. (2014) Biochem Biophys Res Commun., 451(2):208-14; Kam et al. (2007) Vaccine, 25(4):729-40; Jaume et al. (2011) J. Virol., 85(20):10582-97. Coronavirus neutralizing antibodies that bind the spike protein RBD can mediate ADE via Fcγ receptor binding and trigger a conformational change in the spike protein that promotes membrane fusion and infection (Corapi et al. (1992); Hohdatsu et al. (1998); Vennema et al. (1990); (Wang et al. (2014); Kam et al. (2007); Jaume et al. (2011); (Wan et al., (2020) J. Virol., 94(5): e02015-19); (Walls et al. (2019) Cell, 176(5):1026-39 e15).

Correlative clinical data supports a role for ADE worsening both Severe Acute Respiratory Syndrome (SARS) and COVID-19 (Jaume et al. (2011); Cao (2020) Nat. Rev. Immunol., 20(5):269-70; Tetro (2020) Microbes Infect., 22(2):72-73; Yuan et al. (2005) Tissue Antigens, 66(4):291-96; Bicheng Zhang et al. (2020) medRxiv; Zhao et al. (2020) Clin. Infect. Dis., doi: 10.1093/cid/ciaa344);Yuan et al. (2005). The FcγRIIa allele that binds IgG2 equivalently (H131), and therefore might be partially blocked by endogenous IgG2, was protective. IgG1 is the predominant IgG isotype triggered by viral infection. Stated another way, patients homozygous for the R131 FcγRIIa allele, who were therefore more likely to bind antiviral IgG antibodies, had the worst outcomes. This, and the finding that FcγRIIa receptor but not FcγRI or FcγRIIIa, is sufficient for ADE of SARS-CoV in vitro indicate that IgG1 antibodies worsened the course of SARS in susceptible patients via ADE (Juame et al. (2011).

More recently, a study of over 200 COVID-19 patients at Wuhan University found that patients with higher SARS-CoV-2-reactive IgG levels were more likely to experience severe disease than those with lower IgG levels (Bicheng Zhang et al. (2020). Conversely, patients in this study who had higher SARS-CoV-2-reactive IgM levels were more likely to have non-severe disease than those with lower IgM levels. IgG can bind Fcγ receptors but IgM cannot. The study also found that higher neutrophil to lymphocyte ratios correlated with severe disease, particularly in patients with high IgG levels. Neutrophils are known to express high levels of FcγRIIa. The association between higher SARS-CoV-2 IgG levels and more severe disease was confirmed by a second study including more than 170 patients (Zhao et al. (2020)). To summarize these findings, having higher levels of SARS-CoV-2 antibodies that are capable of activating Fcγ receptors is correlated with more severe COVD-19 disease and, conversely, having higher levels of SARS-CoV-2 antibodies that are not capable of activating Fcγ receptors is correlated with less severe COVID-19. A higher proportion of neutrophils, which express FcγRIIa, is also correlated with worse COVID-19, particularly in those patients who had both high levels of SARS-CoV-2 antibodies and a higher proportion of neutrophils. Together, these clinical correlations suggest that ADE may be contributing to the severity of COVID-19, which is invariably characterized by pulmonary inflammation and often ARDS.

The ACE2-Fc fusion polypeptides utilize an Fc domain with markedly attenuated Fcγ receptor binding in order to mitigate the risk of ADE. In some embodiments, two engineered Fc domains that are known to exhibit the least amount of Fcγ receptor binding comprise IgG1 L234A L235A substitutions (IgG1-LALA) and IgG4 S228P L235E substitutions (IgG4-SPLE). IgG4-SPLE, for example, exhibits almost no binding to FcγRIIa, whereas IgG1-LALA does exhibit minimal FcγRIIa binding at high concentrations (Schlothauer et al. (2016) Protein Eng. Des. Sel., 29(10):457-66). In some embodiments, the Fc domain fragment comprises the IgG4-SPLE substitutions to minimize FcγRIIa binding because, as noted above, this is the Fcγ receptor that is most associated with ADE. In some embodiments, an engineered IgG Fc domain in which FcγR binding is markedly attenuated is used. For example, a human IgG4 Fc domain bearing the S228P and L235E point substitutions can be used, wherein the L235E substitution attenuates FcγR binding. FcγRI binding is reduced by several orders of magnitude and there is negligible binding to FcγRIIa, FcγRIIb, and FcγRIII. Binding to the Fc neonatal receptor is preserved, which is for favorable pharmacokinetics. Therefore, the ACE2-Fc fusion polypeptide has low affinity for Fcγ receptors, reducing the risk of ADE.

In some embodiments, the use of IgG4-SPLE will not only mitigate the risk of ADE from the ACE2-Fc fusion polypeptide itself but will also allow the ACE2-Fc fusion polypeptide to interrupt ongoing ADE mediated by endogenous immunopathologic IgG antibodies. In some embodiments, an IgG1 Fc domain may be selected from an IgG1 Fc domain fragment with L234A, L235A, and P329G substitutions; an IgG1 Fc domain fragment with a P329G substitution; an IgG1 Fc domain fragment with L234A and L235A substitutions; an IgG1 Fc domain fragment with a N297D substitution; an IgG1 Fc domain fragment with a N297A substitution; and an IgG1 Fc domain fragment with a N297Q substitution. IgG1 Fc domains may be used to mitigate the risk of ADE and allow the ACE2-Fc fusion polypeptide to interrupt ongoing ADE mediated by endogenous immunopathologic IgG antibodies. At sufficient peripheral tissue concentrations, the ACE2-Fc fusion polypeptide successfully competes with and blocks these endogenous antibodies that bind the SARS-CoV-2 spike protein RBD. This decreases the number of virions bound to an antibody that can promote ADE by binding Fcγ receptors.

Through interrupting the pathophysiologic mechanism of ADE and decreasing the number of virions in the lung by direct competitive inhibition of viral infection, the ACE2-Fc fusion polypeptides encompassed by the present invention prevent, decrease the severity of, and/or treat ARDS in COVID-19 patients.

The ACE2-Fc fusion polypeptides encompassed by the present invention can be conjugated with heterologous agents using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features an ACE2-Fc fusion polypeptide conjugated to a therapeutic moiety, such as an antiviral drug. Conjugated ACE2-Fc fusion polypeptides, in addition to therapeutic utility, can be useful for diagnostically or prognostically to monitor polypeptide levels in samples as part of a clinical testing procedure, e.g., to determine the efficacy of a given treatment regimen. Detection can be facilitated by coupling (i e., physically linking) the ACE2-Fc fusion polypeptide to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include a flag tag, a myc tag, an hemagglutinin (HA) tag, streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the ACE2-Fc fusion polypeptide, is intended to encompass direct labeling of the ACE2-Fc fusion polypeptide by coupling (i.e., physically linking) a detectable substance, such as a radioactive agent or a fluorophore (e.g. fluorescein isothiocyanate (FITC) or phycoerythrin (PE) or Indocyanine (Cy5)) to the ACE2-Fc fusion polypeptide, as well as indirect labeling of the ACE2-Fc fusion polypeptide by reactivity with a detectable substance.

The ACE2-Fc fusion polypeptide conjugates of the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

1. Prophylactic Methods

In one aspect, the invention provides a method for preventing in a subject, a coronavirus infection, by administering to the subject an ACE2-Fc fusion protein that binds to coronaviruses, thereby inhibiting the virus from binding to ACE2 and entry to the cell. Administration of a prophylactic agent can occur prior to the manifestation of symptoms characteristic of a coronavirus infection, such that the infection is prevented or, alternatively, the symptoms of infection are decreased or eliminated. Due to the extended half-life of fusion proteins comprising an Fc fragment, the ACE2-Fc fusion polypeptide can be administered by subcutaneous injection in ambulatory settings. This enables treatment of military service members, veterans, or civilians with COVID-19 in field or community settings, decreases the rate of infectious spread, reduces hospitalization, and allows for prophylaxis of exposed or at-risk individuals.

Additionally, administration of an ACE2-Fc fusion polypeptide can reduce or eliminate antibody dependent enhancement (ADE) if the subject receiving the prophylactic is exposed to and develops an adaptive immune response against the coronavirus.

2. Therapeutic Methods

Another aspect of the invention pertains to methods of treating a subject infected with a coronavirus by administering an ACE2-Fc fusion polypeptide encompassed by the present invention. The fusion polypeptide competitively inhibits the binding of a coronavirus to endogenous ACE2, thereby prohibiting further infection of cells by the virus.

As used herein, the term “agent” and “therapeutic agent” is defined broadly as any compound or composition comprising an ACE2-Fc fusion polypeptide that can be administered to a subject or applied to a cell culture to inhibit binding of a coronavirus to an endogenous ACE2 polypeptide and/or reduce or eliminate antibody dependent enhancement of a coronavirus infection.

Therapeutic methods of the invention involve contacting a coronavirus (e.g., SARS-CoV-2) with an ACE2-Fc fusion polypeptide of the invention, including comprising an ACE2 extracellular domain having an amino acid sequence shown in Table 1, a hinge polypeptide having an amino acid sequence in Tables 2, 3, or 4, or a cysteine-proline, or a proline amino acid hinge, and a Fc domain fragment having an amino acid shown in Table 5.

ACE2-Fc fusion polypeptide of the invention competitively inhibits binding of a coronavirus to endogenous ACE2, and by virtue of the fact that ACE2-Fc fusion polypeptide modulates the amount of viral entry into a cell, it also modulates the total amount of coronavirus activity in a cell (i.e., viral replication). Additionally, using the ACE2-Fc fusion polypeptide at therapeutic concentrations will outcompete endogenously expressed anti-coronavirus antibodies for binding to the virus, thereby reducing or eliminating ADE in the subject.

In one embodiment, the agent inhibits or enhances the interaction of the coronavirus with endogenous ACE2 or other binding partner. In another embodiment, the agent sequesters coronavirus, thereby preventing endogenous antibodies from binding the virus. By reducing or eliminating endogenous antibodies from binding to the coronavirus, antibody dependent enhancement of the infection is reduced or eliminated.

In some embodiments, DF-COV compounds are used as a treatment of coronavirus variants that are resistant to antibodies and/or vaccines. Such variants are now emerging. In publicly available data, the “South African variant” demonstrates some resistance to vaccines, as well as marked resistance to several clinically-relevant monoclonal antibodies, including complete resistance to Eli Lilly's bamlanivimab. Additional data suggests that this is due to the E484K substitution in the receptor binding domain of the viral S-protein, which is also present in the “Brazilian variant.”

These methods can be performed in vitro (e.g., by contacting a cell culture with the agent, wherein the cell culture is infected with a coronavirus at the time of contacting or will be exposed to a coronavirus) or, alternatively, by contacting a bodily fluid with an agent in vivo (e.g., by administering the agent to a subject). As such, the present invention provides methods of treating an individual infected with a coronavirus or at risk of becoming infected with a coronavirus that would benefit from an agent that inhibits viral entry into a cell. In one embodiment, the method involves administering an ACE2-Fc fusion polypeptide that inhibits or reduces antibody dependent enhancement of a coronavirus infection.

The effectiveness of any particular therapeutic agent to treat a coronavirus infection can be monitored by comparing two or more samples obtained from a subject undergoing treatment. In general, a first sample is obtained from the subject prior to beginning therapy and one or more samples during treatment. In such a use, a baseline of expression of cells from subjects having a coronavirus infection (e.g., COVID-19) prior to therapy is determined and then changes in the baseline state of expression of cells from subjects with the coronavirus infection is monitored during the course of therapy. Alternatively, two or more successive samples obtained during treatment can be used without the need of a pre-treatment baseline sample. In such a use, the first sample obtained from the subject is used as a baseline for determining whether the viral load is increasing or decreasing in the subject.

In addition, the ACE2-Fc fusion polypeptides can also be administered in combination other therapies with, e.g., antiviral agents, antimalarials, anti-inflammatories, pain relievers (e.g., acetaminophen, ibuprofen, etc.), and small molecule therapeutics including, but not limited to, remdesivir.

### V. Kits

The present invention also encompasses kits for treating a coronavirus infection. A kit encompassed by the present invention may also include instructional materials disclosing or describing the use of the kit or an ACE2-Fc fusion polypeptide of the disclosed invention in a method of the disclosed invention as provided herein. A kit may also include additional components to facilitate the particular application for which the kit is designed. For example, a kit may additionally contain means of administering the ACE2-Fc fusion polypeptide (e.g., a syringe, sterilized vials, and/or sterilization reageants). A kit may additionally include buffers and other reagents recognized for use in a method of the disclosed invention. Non-limiting examples include agents to reduce non-specific binding, such as a carrier protein or a detergent.

### EXAMPLES

### Example 1: Materials and Methods

### Design and Production of DF-COV Constructs

DF-COV-01 and DF-COV-02 were designed as follows. The amino acid sequence of human angiotensin converting enzyme 2 (ACE2) was obtained from the Universal Protein database (UniProt). For DF-COV-01, the full ACE2 extracellular domain, amino acids 18-730, was used. For DF-COV-02, a subset of the ACE2 extracellular domain, amino acids 18-615, was used. For both DF-COV-01 and DF-COV-02, catalytic histidines 374 and 378 were mutated (H374N and H378N) to render the ACE2 extracellular domain enzymatically inactive. The sequences for these ACE2 extracellular domains were appended to the amino terminus of an amino acid sequence of the human IgG4 hinge region and Fc domain, obtained from UniProt. In both DF-COV-01 and DF-COV-02, the IgG4 hinge region and Fc domain included point substitutions known to decrease FAB-arm exchange, Fcγ receptor binding, and C1q binding: S228P and L234E (hereafter referred to as IgG4-SPLE). The exact hinge region and Fc domain sequences used for DF-COV-01 and DF-COV-02 are listed separately below. To construct DF-COV-01, the appropriate ACE2 extracellular domain listed below was directly appended to the amino terminus of the appropriate hinge region listed below. To construct DF-COV-02, the appropriate ACE2 extracellular domain listed below was appended to the amino terminus of the appropriate hinge region listed below and a single glycine residue was inserted between the ACE2 extracellular domain and the hinge region. The nucleotide sequences encoding DF-COV-01 and DF-COV-02 were obtained by computational reverse-transcription and codon optimization. A nucleotide sequence encoding the signal peptide MEWSWVFLFFLSVTTGVHS was appended immediately 5′ to both the heavy and light chain nucleotide sequences.

The nucleotide sequences were synthesized and cloned into the pLEV123 expression vector (LakePharma, Inc.) and the sequence was confirmed by PCR amplification followed by Sanger sequencing. The vector was then amplified by PCR and the amplified DNA was run on an agarose gel for quality assessment and its sequence confirmed again by Sanger sequencing before proceeding to transfection. Protein production and purification was performed by LakePharma's standard operating procedure. In brief, Chinese hamster ovary (CHO) cells were seeded in suspension in a shake flask and were expanded using serum-free chemically defined medium. On the day of transfection, the expanded cells were seeded into a new flask with fresh medium. The expression vector was transiently transfected into the CHO cells. The cells were maintained as a batch-fed culture for fourteen days. The conditioned media from the transient production run was harvested and clarified by centrifugation and filtration. The supernatant was loaded over a Protein A column pre-equilibrated with binding buffer. Washing buffer was passed through the column until the OD280 value (NanoDrop, Thermo Scientific) was measured to be zero. The target protein was eluted with a low pH buffer, fractions were collected, and the OD280 value of each fraction was recorded. Fractions containing the target protein were pooled and filtered through a 0.2 μm membrane filter. The protein concentration was calculated from the OD280 value and the calculated extinction coefficient. Capillary electrophoresis (CE-SDS) analysis of the target protein was performed using LabChip GXII (Perkin Elmer). To confirm a low endotoxin level, duplicate samples of the purified product were quantified using the chromogenic Limulus Amebocyte Lysate method (Endosafe-MCS, Charles River Laboratories).

### SARS-CoV-02 Spike Protein Binding Assay

Binding of DF-COV-01 and DF-COV-02 to the SARS-CoV-2 spike protein (S-protein) was assessed by enzyme-linked immunosorbent assay (ELISA). Corning Costar 96-well ELISA plates were coated overnight at 4° C. with 5 μg/mL of recombinant trimerization-stabilized, prefusion-stabilized, SARS-CoV-2 S-protein (LakePharma, Ref #46328) in carbonate buffer (15 mM Na₂CO₃ and 35 mM NaHCO₃) at pH 9.5. The plate was washed three times in PBS-tween (Phosphate-Buffered Saline pH 7.4 purchased from Gibco and supplemented with 0.05% Tween 20 purchased from Sigma) using a BioTek 405TS Microplate Washer. The plate was then blocked by incubation in 5% weight/volume milk in PBS. The plate was then washed three times in PBS-tween using a BioTek 405TS Microplate Washer. Serial two-fold dilutions of DF-COV-01 or DF-COV-02 in PBS-BSA (PBS with 0.5% bovine serum albumin) were added to appropriate wells of the ELISA plate in triplicate and incubated at room temperature for 30 minutes. The plate was then washed three times in PBS-tween using a BioTek 405TS Microplate Washer. Horseradish peroxidase-conjugated anti-human IgG secondary antibody (Southern Biotech, 2014-05) diluted 1:5000 in PBS-BSA was added to all wells and incubated at room temperature for 15 minutes. The plate was then washed three times in PBS-tween using a BioTek 405TS Microplate Washer. A peroxidase substrate was added (TMB Microwell Peroxidase Substrate System, KPL Inc.), followed by a 50% volume of stop solution of 1 M phosphoric acid. Optical density at 450 nm, representing the degree of DF-COV binding to the SARS-CoV-2 S-protein, was determined for each well using a Molecular Devices SpectraMax M3 plate reader.

### FcγRIIa Binding Assay

Binding of DF-COV-01 and DF-COV-02 to recombinant human FcγRIIa was assessed by ELISA. Corning Costar 96-well ELISA plates were coated overnight at 4° C. with 1 μg/mL of recombinant human Fcγ RIIA/CD32a (H167) protein (R&D Systems, 9595-CD-050) in pH 9.5 carbonate buffer. The plate was washed three times in PBS-tween using a BioTek 405TS Microplate Washer. The plate was then blocked by incubation in 5% weight/volume milk in PBS. The plate was then washed three times in PBS-tween using a BioTek 405TS Microplate Washer. Serial two-fold dilutions of DF-COV-01, DF-COV-02, or human ACE2-IgG1 Fc (LakePharma, Ref #46672 & #46673) in PBS-BSA were added to appropriate wells of the ELISA plate in triplicate and incubated at room temperature for 30 minutes. The plate was then washed three times in PBS-tween using a BioTek 405TS Microplate Washer. Horseradish peroxidase-conjugated anti-human IgG F(ab′)2 secondary antibody (Southern Biotech, 2042-05) diluted 1:5000 in PBS-BSA was added to all wells and incubated at room temperature for 15 minutes. The plate was then washed three times in PBS-tween using a BioTek 405TS Microplate Washer. A peroxidase substrate was added (TMB Microwell Peroxidase Substrate System, KPL Inc.), followed by a 50% volume of stop solution of 1 M phosphoric acid. Optical density at 450 nm, representing the degree of construct binding to FcγRIIa was determined for each well using a Molecular Devices SpectraMax M3 plate reader.

### ACE2 Enzymatic Activity Assay

Assessment of ACE2 enzymatic activity was performed by a fluorometric enzyme activity assay. Two-fold serial dilutions of DF-COV-01, DF-COV-02, or human ACE2-IgG1 Fc (LakePharma, Ref #46672 & #46673) were diluted in 100 μL of reaction buffer containing 0.1% BSA, 1 M NaCl, 75 mM Tris, 0.5 mM ZnCl2, pH 7.5 in 96-well Corning Costar plates. Mca-YVADAPK(Dnp)-OH Fluorogenic Peptide Substrate (R&D Systems, ES007) was added at a concentration of 50 μM. Reaction progress was monitored by fluorescence (excitation 322 nm, emission 381 nm) using a Molecular Devices SpectraMax M3 plate reader maintained at 37 degrees Celsius. The change in fluorescence was used to calculate reaction velocity. Reaction velocities were used to compare the ACE2 enzymatic activity of DF-COV-01 and DF-COV-02 with that of wild type ACE2-IgG1 Fc.

### Pseudotyped Virus Neutralization Studies

Pseudotyped SARS-CoV and SARS-CoV-2 were generated using a replication-deficient HIV-1 backbone that contained a mutation to prevent HIV envelope glycoprotein expression and a luciferase gene to direct luciferase expression in target cells. HEK293-T cells were co-transfected with two plasmids: one an expression vector for either the SARS-CoV or the SARS-CoV-2 spike protein and the other bearing the Env-defective HIV-1 genome. Supernatant containing virus particles was harvested 48 hours after transfection, concentrated using Centricon 70 concentrators, and stored frozen at −80° C. Target cells were generated by transiently transfecting 293T cells with a human ACE2 expression vector (pcDNA-hACE2). ACE2-transfected 293T cells were cells were used as target cells 24 hours after transfection. A titration of pseudovirus was performed on 293T cells transiently transfected with human ACE2 receptor to determine the volume of virus need to generate 50,000 counts per second (cps) in the infection assay. The appropriate volume of pseudovirus was pre-incubated with serial dilutions of DF-COV-01, DF-COV-02, or an irrelevant Fc fusion for 1 hour at room temperature before adding to 293T cells expressing human ACE2. Media containing pseudovirus was replaced by the fresh complete media 24 hours later. After additional 24 hours, the infection was quantified by luciferase detection with BrightGlo luciferase assay (Promega) and read in a Victor3 plate reader (Perkin Elmer).

### Pharmacokinetic Studies

Syrian hamsters were weighed and then injected subcutaneously with 8 mg/kg of DF-COV-01 or DF-COV-02. Between 50 μL and 100 μL of venous blood was subsequently obtained from the saphenous vein at 4 hours, 8 hours, 24 hours, 48 hours, and 72 hours following injection. Serum from these saphenous blood samples was frozen for later analysis. The concentration of DF-COV-01 and DF-COV-02 in the serum samples was determined by ELISA. Corning Costar 96-well ELISA plates were coated overnight at 4° C. with 5 μg/mL of recombinant trimerization-stabilized, prefusion-stabilized, SARS-CoV-2 S-protein (LakePharma, Ref #46328) in carbonate buffer at pH 9.5. The plate was washed three times in PBS-tween using a BioTek 405TS Microplate Washer. The plate was then blocked by incubation in 5% weight/volume milk in PBS. The plate was then washed three times in PBS-tween using a BioTek 405TS Microplate Washer. Serial two-fold dilutions of serum sample in PBS-BSA (PBS with 0.5% bovine serum albumin) were added to appropriate wells of the ELISA plate in triplicate and incubated at room temperature for 30 minutes. This was performed in parallel with samples of DF-COV-01 or DF-COV-02 at a known concentration to define a standard curve. The plate was then washed three times in PBS-tween using a BioTek 405TS Microplate Washer. Horseradish peroxidase-conjugated anti-human IgG secondary antibody (Southern Biotech, 2014-05) diluted 1:5000 in PBS-BSA was added to all wells and incubated at room temperature for 15 minutes. The plate was then washed three times in PBS-tween using a BioTek 405TS Microplate Washer. A peroxidase substrate was added (TMB Microwell Peroxidase Substrate System, KPL Inc.), followed by a 50% volume of stop solution of 1 M phosphoric acid. Optical density at 450 nm, representing the degree of DF-COV binding to the SARS-CoV-2 S-protein was determined for each well using a Molecular Devices SpectraMax M3 plate reader. OD 405 of serum dilutions that fell within the linear range of the standard curve were utilized to calculate the serum concentration of DF-COV-01 or DF-COV-02 at each time point, after accounting for the appropriate dilution factor.

### Antibody-Dependent Enhancement Assay

The ability of reagents to promote infection of target cells that express FcγRIIa but do not express ACE2 can be used as a model of their ability to promote antibody-dependent enhancement (ADE). Pseudotyped SARS-CoV-2 virus was produced and purified as described above, except that the replication-deficient HIV-1 vector contained the gene for green fluorescent protein (GFP) rather than luciferase as a reporter. To assess the ability of reagents to promote ADE, pseudotyped virus is incubated with serial dilutions of the appropriate reagent: a SARS-CoV-02 receptor-binding domain (RBD) specific neutralizing antibody (Sino Biological, #40592-MM57 or Genscript, A02038), an irrelevant isotype control antibody, DF-COV-01, DF-COV-02, or an irrelevant Fc fusion for 1 hour at room temperature. This is then added to cultures of Jurkat cells transformed to express human FcγRIIa and that have been demonstrated by flow-cytometry to not express ACE2 (Promega). After a 24-hour incubation at 37° C. in an atmosphere of 5% CO₂, the media containing pseudovirus is replaced with fresh complete media. After an additional 24 hours of incubation under the same conditions, infection is quantified by determining GFP expression using quantitative immunofluorescence microscopy using a Celigo Imaging Cytometer. Infection, as measured by GFP expression, is indicative of antibody-dependent enhancement (viral entry in to Fc-receptor expressing target cells that is mediated by an anti-viral antibody).

To assess the ability of DF-COV-01 and DF-COV-02 to inhibit ADE promoted by a SARS-CoV-2 RBD-specific antibody, pseudotyped virus is produced and purified as described above and was incubated with varying molar ratios of the SARS-CoV-02 RBD-specific antibody and either DF-COV-01, DF-COV-02, or an irrelevant Fc fusion for 1 hour at room temperature. As above, this is added to cultures of Jurkat cells transformed to express human FcγRIIa and that have been demonstrated by flow-cytometry to not express ACE2. After a 24-hour incubation at 37° C. in an atmosphere of 5% CO₂, the media containing pseudovirus is replaced with fresh complete media. After an additional 24 hours of incubation under the same conditions, infection is quantified by determining GFP expression using quantitative immunofluorescence microscopy using a Celigo Imaging Cytometer. Infection, as measured by GFP expression, is indicative of ADE, and the degree of reduction of infection in conditions that included DF-COV-01 or DF-COV-02 when compared with conditions that included the irrelevant Fc fusion serves as a metric of ADE inhibition.

In addition to assessing viral entry as a metric of ADE, FcγRIIa activation by immune complexes of pseudotyped SARS-CoV-2 opsonized with either SARS-CoV-02 RBD-specific antibody, isotype control antibody, DF-COV-01, DF-COV-02, or an irrelevant Fc fusion protein can be assessed. DF-COV-01 and DF-COV-02 can also be assessed for their ability to block SARS-CoV-02 RBD-specific antibody opsonization of pseudotyped virus and mediate FcγRIIa activation. SARS-CoV-2 pseudovirus expressing a GFP reporter is produced as described above and incubated with serial dilutions of SARS-CoV-02 RBD-specific antibody, isotype control antibody, DF-COV-01, DF-COV-02, or an irrelevant Fc fusion protein for 1 hour at room temperature. For experiments assessing the ability of DF-COV-01 or DF-COV-02 to block viral opsonization and resultant FcγRIIa activation, pseudovirus is incubated with varying molar ratios of the SARS-CoV-02 RBD-specific antibody and either DF-COV-01, DF-COV-02, or an irrelevant Fc fusion for 1 hour at room temperature. FcγRIIa activation in each of these experimental conditions is assessed using the Promega FcγRIIa-H ADCP Bioassay (G9901), which is performed according to the kit's recommended protocol. Luminescence, which serves as a metric of FcγRIIa activation, is determined for each well using a Molecular Devices SpectraMax M3 plate reader.

### In Vivo Efficacy Studies

Male and female hamsters between 6 and 8 weeks of age were infected with between 103 and 105 plaque forming units per milliliter (pfu/mL) of SARS-CoV-2 in 100 μL of conditioned media (Vero passage 3 of USA-WA01-2020 strain, BEI Resources) by intranasal instillation under ketamine-xylazine anesthesia. On the same day hamsters were weighed and injected subcutaneously with 8 mg/kg of DF-COV-01, DF-COV-02, or an equivalent volume of PBS as a vehicle control. Oropharyngeal swabs were collected into viral transport medium once daily beginning immediately prior to challenge and extending for 3 days. Euthanasia and necropsy were performed on half of the animals at day 3 and half on day 7 post-challenge, the days with the highest viral titer and most severe lung pathology, respectively. Multiple tissues, including lung, are collected for virus titration and histopathology at these time points. Viral titers were determined by a Vero cell monolayer plaque assay. Vero cell monolayers were grown to 80% confluency in 1 mL Eagle's MEM media supplemented with 10% fetal bovine serum in six-well Costar tissue culture treated plates. A series of 10-fold dilutions of sample in viral transport medium were prepared in 96-well plates and 100 μL of each sample was added to separate appropriate wells containing Vero cell monolayers. After rocking each plate to evenly distribute the inoculum, these cultures were then incubated for 60-90 minutes at 37° C. in an atmosphere of 5% CO₂. Medium containing the inoculum was then removed and the monolayers were washed with PBS. An agarose overlay containing equal volumes of Eagle's MEM mediate supplemented with 10% fetal bovine serum and boiled 1% agarose solution pre-cooled to 42° C. was added over each washed Vero monolayer, cooled for 15 minutes, and then incubated for 4 days at 37° C. in an atmosphere of 5% CO₂. Plates were then stained with a 1:10,000 dilution of neutral red in a complete MEM media/agarose overlay prepared similarly. After overnight incubation at 37° C. in an atmosphere of 5% CO₂, plaques were counted. Dilution of the inoculum was used to transform the plaque count in to the number of plaque forming units per milliliter (pfu/mL) of the initial inoculum sample.

### Example 2: Assessment of Binding of ACE2-Fc Fusion Polypeptides to SARS-CoV-2 in Vitro

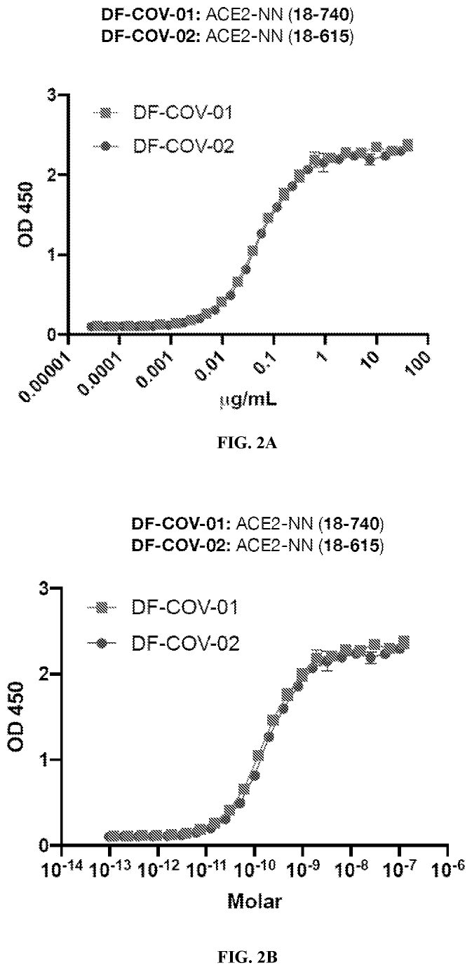

The ability of an ACE2-Fc fusion polypeptide to bind a recombinant SARS-CoV-2 spike protein (produced by LakePharma) was assessed in vitro. Briefly, binding of DF-COV-01 and DF-COV-02 to a recombinant SARS-CoV-2 spike protein was detected by enzyme-linked immunosorbent assay (ELISA). The SARS-CoV-2 spike protein was immobilized on a 96-well plate. The plate was then blocked with milk protein, washed, and serial two-fold dilutions of DF-COV-01 or DF-COV-02 were added to appropriate wells. The plate was again washed and binding of DF-COV-01 or DF-COV-02 was detected by a peroxidase-labeled anti-human IgG secondary antibody. After addition of a colorimetric peroxidase substrate, binding was measured using an optical density plate reader. Binding (optical density) was plotted as a function of concentration of DF-COV-01 or DF-COV-02 to generate a binding curve (FIGS. 2A and 2B). Binding can be compared with that of a previously produced wild type ACE2-Fc fusion. A non-specific Fc fusion (human PD-L1-Fc) can serve as a negative control.

### Example 3: Assessment the Enzymatic Activity of ACE2-Fc Fusion Polypeptides In Vitro

Assessment of the ACE2 enzymatic activity of DF-COV-01 and DF-COV-02 is performed by a fluorometric enzyme activity assay utilizing a commercially available fluorogenic ACE2 substrate (Mca-YVADAPK(Dnp)-OH). The assay is performed as previously described (Imai et al. (2005) Nature, 436(7047):112-6). Briefly, the reaction are performed in a pH 7.5 buffer conducive to ACE2 enzymatic activity containing 0.1% BSA, 1 M NaCl, 75 mM Tris, 0.5 mM ZnCl₂ at 37° C. The change in fluorescence indicates enzymatic activity and is monitored using a fluorescence plate reader. ACE2 enzymatic activity of DF-COV-01 and DF-COV-02 is compared with that of a previously produced wild type enzymatically active ACE2-Fc fusion. A non-specific Fc fusion serves as a negative control.

For enzymatic activity, reaction velocity (derivative of fluorescence change) is compared for varying concentrations of DF-COV-01 or DF-COV-02 or controls.

### Example 4: DF-COV-01 and DF-COV-02 Inhibit Infection of Human Alveolar Type 2 Cells Derived from iPSCs (iAT2 Cells) by Native SARS-CoV-2 Virus and SARS-CoV-2 Pseudovirus In Vitro

A model of human type 2 alveolar cell infection with native SARS-CoV-2 virus model utilizes iAT2 cells, which are alveolar type 2 cells (AT2) differentiated from induced pluripotent stem cells (iPSC). These cells and air-liquid interface 2D cultures are prepared as previously described (Kristine et al. (2020) bioRxiv, biorxiv.org/content/10.1101/2020.06.03.132639v1).

### Example 5: In Vitro Prophylactic and Therapeutic Treatment of SARS-CoV-2 Infected iAT2 Cells

Two studies are performed: (1) a prophylactic study, where DF-COV-01 or DF-COV-02 is administered at the same time as SARS-CoV-2 infection and also maintained in the iAT2 culture thereafter and (2) a therapeutic study, where DF-COV-01 or DF-COV-02 is administered to the iAT2 culture one day after the SARS-CoV-2 infection and maintained in the iAT2 culture thereafter. For both experiments, viral titer is quantified by quantitative RT-PCR at day 4 following infection. The viral titer measurably increases from day 1 to day 4, without treatment. Treatments in each of these two studies include two-fold serial dilutions of either DF-COV-01 or DF-COV-02 or a non-specific Fc-fusion utilized as a negative control.

Viral titer in culture is measured by quantitative RT-PCR and infection of iAT2 pneumocytes is assessed by quantitative immunofluorescence microscopy for viral N protein. Two separate neutralization curves are generated based on the viral titer data and N protein expression is quantified by immunofluorescence. Key data that are calculated from each of these curves are: (1) the IC50 for DF-COV viral neutralization in this system and (2) the concentration at which DF-COV completely, or nearly completely, neutralizes virus.

### Example 6: DF-COV-Mediated Inhibition of SARS-CoV-2 Pseudoviral Infection of ACE2-Transduced 293 Cells

Standard pseudovirus neutralization studies were performed as detailed below to compare viral neutralization between ACE2-Fc fusion polypeptides comprising an inactive ACE2 domain and an Fc domain fragment that does not bind to an Fcγ receptor (e.g., a FcγIIa receptor) and RBD antibodies and PDL-1 Fc polypeptide.

Pseudovirus expressing the full-length spike protein of SARS-CoV-2 is used in order to assess neutralization by DF-COV-01 or DF-COV-02 under BioSafety Level (BSL)-2 conditions. The pseudovirus was generated using a replication deficient HIV-1 backbone that expresses firefly luciferase. Virus lacking the SARS-CoV-2 spike protein was generated as a control. The pseudovirus was used to infect HEK293A cells transfected with human ACE2 (293A/ACE2). Two-fold serial dilutions of the pseudovirus were incubated with varying concentrations of DF-COV-01 or DF-COV-02 or a non-specific Fc-fusion as a control. Following infection, cells were incubated and luciferase assays are performed on lysates of harvested cells to quantify viral infection.

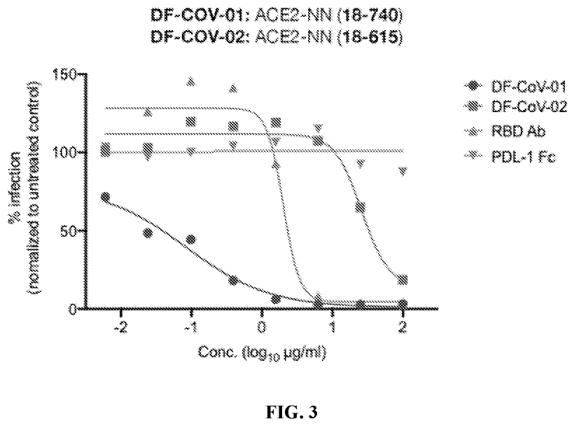

Luminescence, as a surrogate of pseudoviral infection, was plotted as a function of concentration of DF-COV-01 or DF-COV-02 or relevant controls to generate a viral neutralization curve (FIG. 3). The IC50 for DF-COV-01 or DF-COV-02 viral neutralization in this system and the concentration at which DF-COV-01 or DF-COV-02 completely, or nearly completely, neutralized virus were calculated using this curve.

### Example 7: Comparison of DF-COV Binding to FcγRIIa and ACE2-Fc Binding to an IgG1 Fc

The FcγRIIa binding of an ACE2-Fc fusion polypeptide is compared to that of a previously made ACE2-Fc with a human IgG1 Fc domain using surface plasmon resonance as previously described (Schlothauer et al. (2016) Protein Eng. Des. Sel., 29(10):457-66). In brief, a commercially available recombinant His-labeled FcγRIIa extracellular domain (R&D Systems) is immobilized on a CM5 Biacore chip that is pre-coated with an anti-His capturing antibody using an established kit protocol. During the Biacore analysis His-labeled FcγRIIa at a concentration of 100 nM is applied in HBS-P+ running buffer. Subsequently, DF-COV-01 or DF-COV-02 or ACE2-Fc with an IgG1 Fc is applied to the hip at a concentration of 100 nM. Association and dissociation phases are monitored by standard protocol and binding constants are calculated by the Biacore evaluation software and utilized to compare the relative affinities of FcγRIIa binding for DF-COV and an ACE2-Fc with an IgG Fc domain. This protocol can be modified to use ELISA analysis in place of surface plasmon resonance.

### Example 8: Comparison of DF-COV Binding to FcγRIIa and ACE2-Fc Binding to a Polyclonal Human IgG

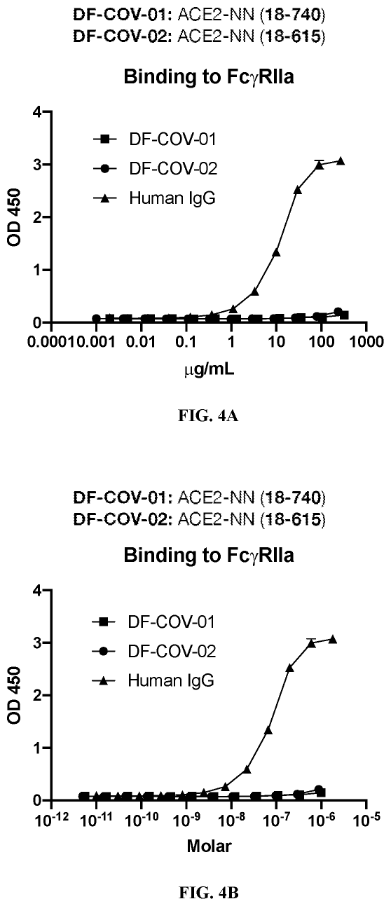

The FcγRIIa binding of an ACE2-Fc fusion polypeptide was compared to that of a polyclonal human IgG by ELISA. In brief, recombinant human FcγRIIa (R&D Systems) was immobilized on an ELISA plate. After blocking the ELSIA plate and washing 3 times in PBS-tween using a BioTek 405TS Microplate Washer, serial dilutions of DF-COV-01, DF-COV-02, or polyclonal human IgG (BioXCell) in PBS-BSA were applied to appropriate wells in triplicate and incubated at room temperature for 30 minutes. After washing the ELISA plate 3 times in PBS-tween using a BioTek 405TS Microplate Washer, all wells were incubated with horseradish peroxidase-conjugated anti-human IgG F(ab′)2 secondary antibody (Southern Biotech) diluted 1:5000 in PBS-BSA for 15 minutes at room temperature. The plate was then washed three times in PBS-tween using a BioTek 405TS Microplate Washer. A peroxidase substrate was added (TMB Microwell Peroxidase Substrate System, KPL Inc.), followed by a 50% volume of stop solution of 1 M phosphoric acid. Optical density at 450 nm, representing the degree of DF-COV or human IgG binding to FcγRIIa was determined for each well using a Molecular Devices SpectraMax M3 plate reader (FIG. 4A and FIG. 4B).

### Example 9: DF-COV-01 and DF-COV-02 Blocks ADE In Vitro

To assess the ability of DF-COV-01 and DF-COV-02 to block ADE, a model system is employed that is similar to the one previously described that demonstrated ADE induced by a MERS-CoV neutralizing antibody in vitro (Wan et al. (2020)). It is first determined whether neutralizing antibody against the SARS-CoV-2 RBD can promote ADE in vitro. To accomplish this, commercially available Jurkat cells that express FcγRIIa (Promega) are used as target cells. Jurkat do not express ACE2 based on a search of T-cell RNAseq data using the Human Protein Atlas (www.proteinatlas.org/ENSG00000130234-ACE2/tissue/T-cells), therefore these cells should not be susceptible to infection by SARS-CoV-2 pseudovirus unless infection is mediated by ADE. These cells are cultured in the presence of SARS-CoV-2 pseudovirus containing a green fluorescence protein (GFP) reporter. GFP expression in the Jurkat cell indicates viral entry. To these co-cultures varying concentrations are added of SARS-CoV-2 neutralizing antibodies that are known to bind to the RBD of the SARS-CoV-2 spike protein. There are several SARS-CoV-2 RBD-specific neutralizing antibodies that are now commercially available (www.genscript.com/antibody/A02038-SARS_CoV_2_Spike_S1_Antibody_HC2001_Human_Chimeric.html; www.sinobiological.com/antibodies/cov-spike-40592-mm57), including some with human IgG1 Fc domains. The ability of these antibodies to induce ADE through FcγRIIa-mediated viral entry is assessed by measuring GFP fluorescence in the Jurkat target cells using flow cytometry. Various commercially available antibodies can be assayed in this system. Comparison is made to a control samples with FcγRIIa Jurkat cells alone or FcγRIIa Jurkat cells incubated with pseudovirus but not antibody.

Use of these commercially available FcγRIIa Jurkat cells has an added benefit because they have been engineered to function as reporters of FcγRIIa activation. FcγRIIa clustering by immune complexes triggers expression of a luciferase reporter in these cells. Therefore, in addition to assessing viral entry by measuring GFP expression, FcγRIIa activation by immune complexes of SARS-CoV-2 pseudovirus with neutralizing antibodies can also be measured. Comparison can be made to control samples with FcγRIIa Jurkat cells alone or FcγRIIa Jurkat cells incubated with pseudovirus but not antibody. FcγRIIa-dependent viral entry, FcγRIIa activation by immune complexes, or both, can be reliably measured. This system is used to show that treatment with DF-COV-01 and DF-COV-02 can result in less ADE (measured by viral entry and/or FcγRIIa activation) than a similar premade ACE2-Fc with an IgG1 Fc domain and can block ADE induced by neutralizing antibodies specific for the SARS-CoV-2 spike protein RBD.

Pseudoviral entry is measured by GFP expression in the target cells using flow cytometry. Activation of FcγRIIa is measured by luminescence of these target cells, which are engineered as a FcγRIIa reporter, using a plate reader.

### Example 10: Determining Efficacy of DF-COV-01 and DF-COV-02 in Reducing Viral Titer and Improving Lung Pathology in an In Vivo Model of SARS-CoV-2 Infection

The ability of DF-COV-01 and DF-COV-02 to reduce SARS-CoV-2 viral infection is assessed in a hamster infection challenge model that displays many of the features of human disease. To validate this model, approximately 50 hamsters were challenged intranasally with SARS-CoV-2 in a BSL-3 animal facility. Clinical disease is mild with transient fevers and lethargy, but no serious manifestations. Oral swabs consistently contain infectious virus on day 3 post-challenge, providing a means of quantifying virus shedding. Organ burdens of virus are high in the respiratory tract on day 3, but not detected on day 7 or 21 post-challenge. Table 9 provides titers in tissues and oropharyngeal swabs from 10 hamsters on day 3 post-challenge.

TABLE 9
Titers

| PFU/gram or swab | Lung | Turbinate | Oral Swab |
|---|---|---|---|
| Mean | 4.13E+06 | 4.32E+07 | 8.56E+02 |
| Minimum | 5.70E+05 | 2.30E+07 | 3.10E+02 |
| Maximum | 6.90E+06 | 5.50E+07 | 1.50E+03 |

Histopathologic evaluation revealed a moderately severe acute bronchointerstitial pneumonia on day 3 post-challenge that was more severe on day 7 despite a lack of infectious virus. These lesions have been highly consistent and experiments are underway to better characterize the resolution of pulmonary lesion (e.g. pulmonary fibrosis).

Male and female hamsters between 6 and 8 weeks of age are infected with SARS-CoV-2 (Vero passage 3 of USA-WA01-2020 strain originally from BEI Resources) by intranasal instillation under ketamine-xylazine anesthesia of between 103 and 105 pfu of virus in 100 μl volume. Animals are closely monitored clinically beginning on the day before viral challenge with body weight recorded daily, body temperatures recorded from subcutaneously implanted Life Chips (Destron-Fearing) twice daily, and clinical assessment and scoring (4-point scale) twice daily. This monitoring and physiologic data is used to assess toxicity. SARS-CoV-2 infection is subclinical in hamsters, thus no deviation is expected from normal in the clinical assessment and scoring from infection alone.

To gather data for study outcomes, oropharyngeal swabs are collected into viral transport medium once daily beginning immediately prior to challenge and extending for 3 days. Euthanasia and necropsy are performed in separate experiments on days 3 and 7 post-challenge, the days with the highest viral titer and most severe lung pathology, respectively. Multiple tissues, including lung, are collected for virus titration and histopathology at these time points.

DF-COV-01 or DF-COV-02 is administered subcutaneously concurrently with viral challenge and in three treatment groups with escalating doses of DF-COV-01 or DF-COV-02 in each treatment group. Dosages are calculated to achieve a specific concentration of drug in the extracellular fluid of the animal, assuming a 60% extravascular and 40% intravascular distribution, in keeping with other receptor Fc-fusions and antibodies. Specific concentrations targeted for the treatment groups are (1) equivalent to the IC50 of DF-COV-01 and DF-COV-02 for pseudovirus neutralization in vitro, (2) equivalent to concentration required to achieve complete, or near complete neutralization of pseudovirus in vitro, and (3) some multiple (e.g., between 2×-10×) of the concentration required to achieve complete, or near complete neutralization of pseudovirus in vitro. Each treatment group consists of six animals. A control group of six animals treated with saline, as a vehicle control, are included in each experiment. The experiment is repeated with day 3 and day 7 endpoints. Day 3 endpoint experiments are used to assess the effect of DF-COV-01 and DF-COV-02 on viral titer. Day 7 endpoint experiments assess the effect of DF-COV-01 and DF-COV-02 on lung pathology. Nasal turbinid, oropharyngeal, and lung viral titer are compared between treatment groups and control groups at day 3. Day 3 and day 7 lung histopathology in treatment and control groups are assessed by a board-certified veterinary pathologist.

The data are subjected to a log 10 transformation to control variability. For each experiment, 6 animals are randomized to each treatment group as detailed above (four groups: low DF-COV-01 or DF-COV-02 dose, medium DF-COV-01 or DF-COV-02 dose, high DF-COV-01 or DF-COV-02 dose, and vehicle control) and assume equal variance in each group. A one-way analysis of variance (ANOVA) is conducted, testing at the 0.10 two-sided significance level and using Dunnett's approach to compare each of the active agents to vehicle control to efficiently adjust the p-values for the multiplicity of testing. This study has greater than 90% power to identify at least one treatment group that differs from control.

Twice daily clinical assessment scores can assess for toxicity. The score is transformed to a binary variable via thresholding. With 6 mice per group, there is 83% power to detect toxicity that occurs in 88% of the treated animals compared to 12% of the controls. Such a comparison is based on the Fisher exact test, conducted at the 0.10 two-sided significance level.

### Example 11: Evaluation of Toxicity of DF-COV-01 and DF-COV-02 in Non-Human Primates

A standard 14-day toxicity study design as outlined in Table 10, with blood sampling to measure circulating drug levels. Both subcutaneous and intravenous routes of administration are utilized, in separate groups, in order to provide safety data to preserve the option of including an intravenous administration arm in addition to a subcutaneous administration arm in clinical trials.

TABLE 10

| Treatment | Endpoint | Recovery | |
|---|---|---|---|
| Group | group | group | Treatment |
| IV placebo | 3 male + 3 | 3 male + 3 | Daily administration of |
| | female | female | indicated treatment for 14 |
| IV low dose | 3 male + 3 | 3 male + 3 | days. Blood samples for |
| | female | female | measurement of circulating |
| IV medium dose | 3 male + 3 | 3 male + 3 | drug are taken on days 1 and |
| | female | female | 14. Endpoint groups are |
| IV high dose | 3 male + 3 | 3 male + 3 | terminated on day 15 for |
| | female | female | histopathological assessment |
| Subcutaneous | 3 male + 3 | 3 male + 3 | of toxicity. Recovery groups |
| placebo | female | female | are observed for an additional |
| Subcutaneous | 3 male + 3 | 3 male + 3 | 7 days, and are terminated on |
| low dose | female | female | day 22 for histological |
| Subcutaneous | 3 male + 3 | 3 male + 3 | assessment of resolution of |
| medium dose | female | female | any observed toxicity. |
| Subcutaneous | 3 male + 3 | 3 male + 3 | |
| high dose | female | female | |

### Example 12: DF-COV-02 has Better Tissue Exposure than DF-COV-01

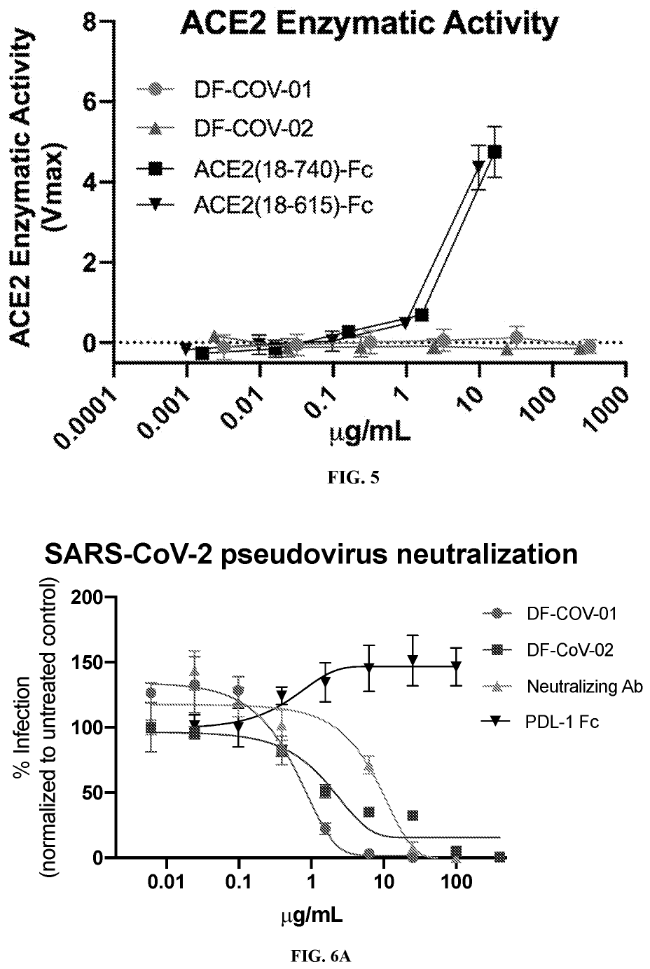

The enzymatic activities of DF-COV-01 and DF-COV-02 were compared with the enzymatic activities of corresponding ACE2-Fc fusions containing wild-type ACE2 extracellular domains. The results indicated that while the matched wild-type ACE2-Fc fusions exhibit enzymatic activity, DF-COV-01 and DF-COV-02 exhibit no ACE2 enzymatic activity (FIG. 5).

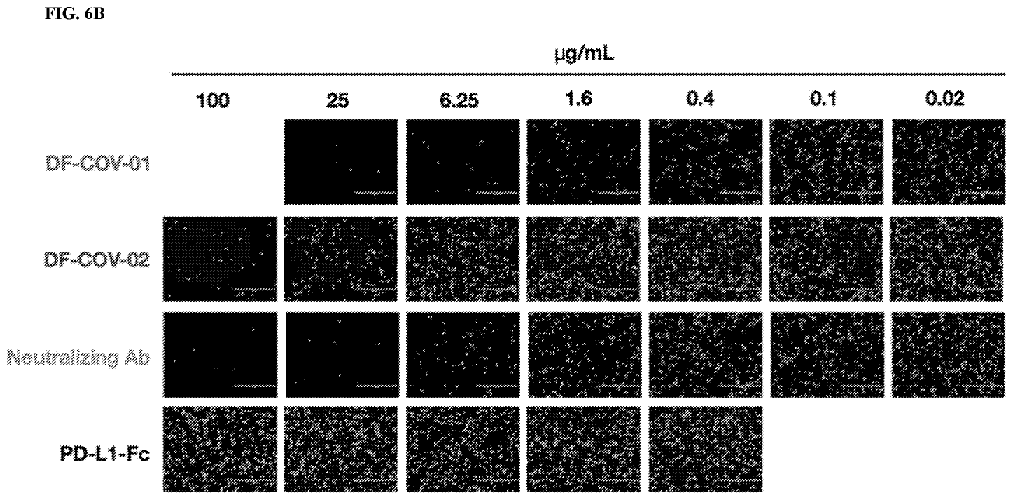

The ability of DF-COV-01 and DF-COV-02 to neutralize pseudotyped SARS-CoV-2 viral particles was characterized using viral particles containing both luciferase and GFP reporter genes and using ACE2-expressing 293T cells as targets. Both the luciferase expression (FIG. 6A) and the GFP expression (FIG. 6B) results indicated that both DF-COV-01 and DF-COV-02 inhibit viral entry in to ACE2-expressing 293T cells, with DF-COV-01 neutralizing more potently than DF-COV-02.

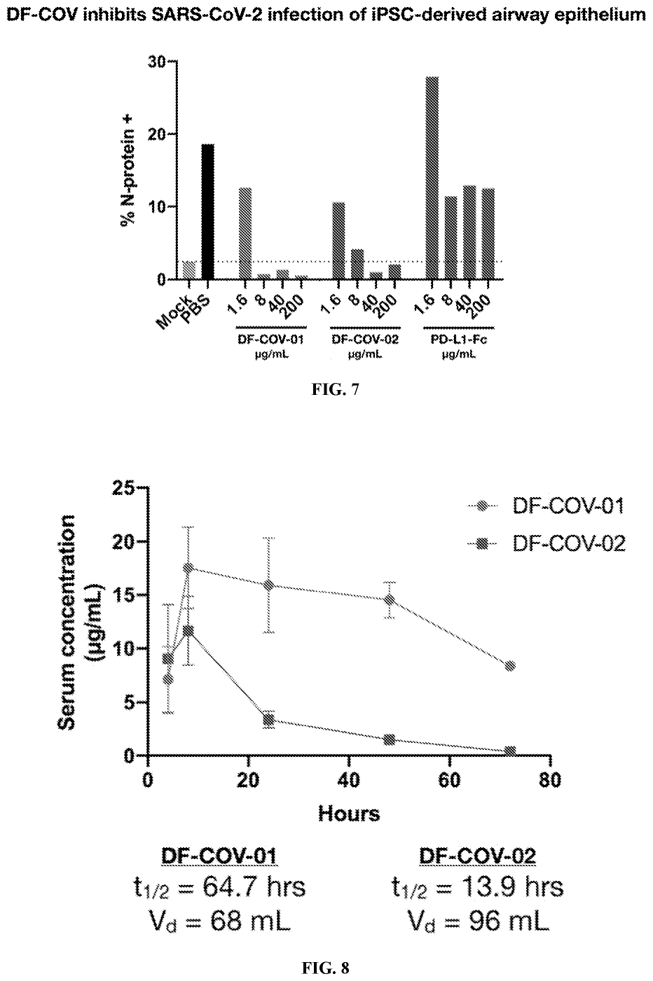

Even though DF-COV-01 has better in vitro viral neutralization activity than DF-COV-02, as described above, surprisingly DF-COV-02 has better activity against SARS-CoV-2 in vivo than DF-COV-01 in certain experiments (FIG. 7). This is believed to be due to the better peripheral tissue penetration of DF-COV-02, as evidenced by the larger volume of distribution calculated from the pharmacokinetic curves in FIG. 8. Thus, large ACE2-Fc fusions that utilize the full ACE2 extracellular domain, such as DF-COV-01, are inferior in some experiments to smaller ACE2-Fc fusions that have better peripheral tissue penetration and thus better drug exposure in tissues where viral neutralization is needed. Such tissue penetration may be useful in some situations.

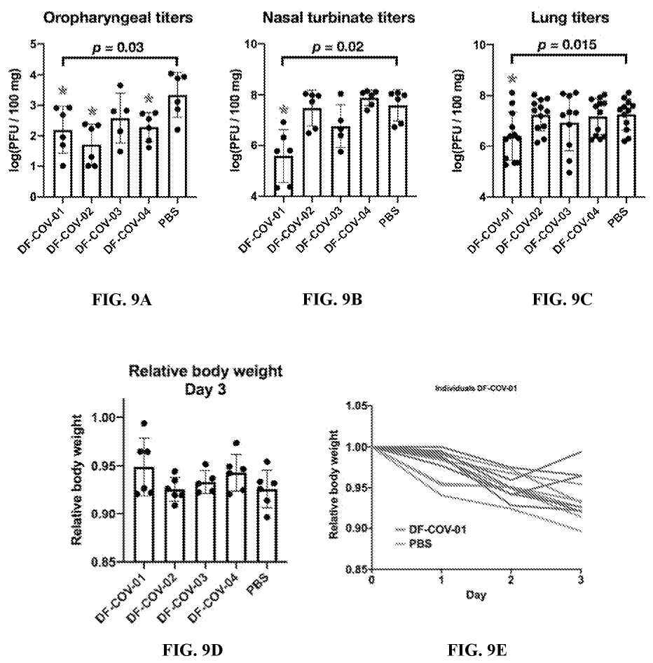

The activity of DF-COV-01, DF-COV-02, DF-COV-03, and DF-COV-04 against the SARS-CoV-2 virus were further characterized in vivo. In connection with that, FIG. 9A compares the oropharyngeal viral titers, FIG. 9B compares the nasal turbinate viral titers, FIG. 9C compares the lung titers, FIG. 9D compares the body weights at a particular day, and FIG. 9E compares the body weights over time. DF-COV-01 reduced oropharyngeal titer, nasal turbinate titer, and lung titer, and it also protected against weight loss, which is a manifestation of SARS-CoV-2 infection in hamsters. In line with these experimental results, in some embodiments, DF-COV-01 is a preferred construct.

### Example 13: Additional Constructs, DF-COV-03 and DF-COV-04, and their Properties

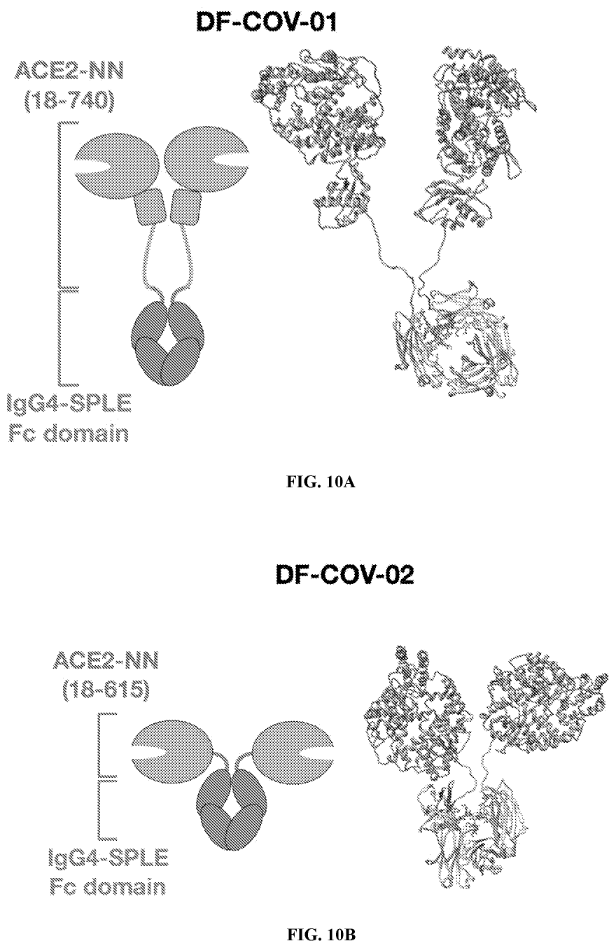

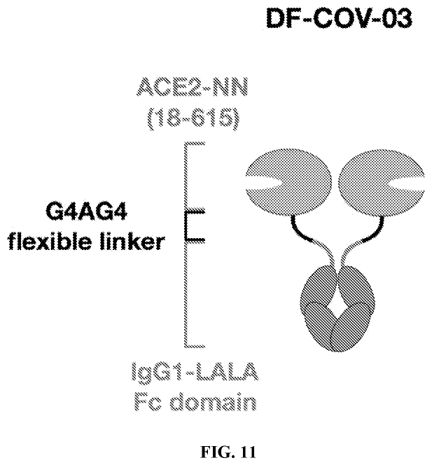

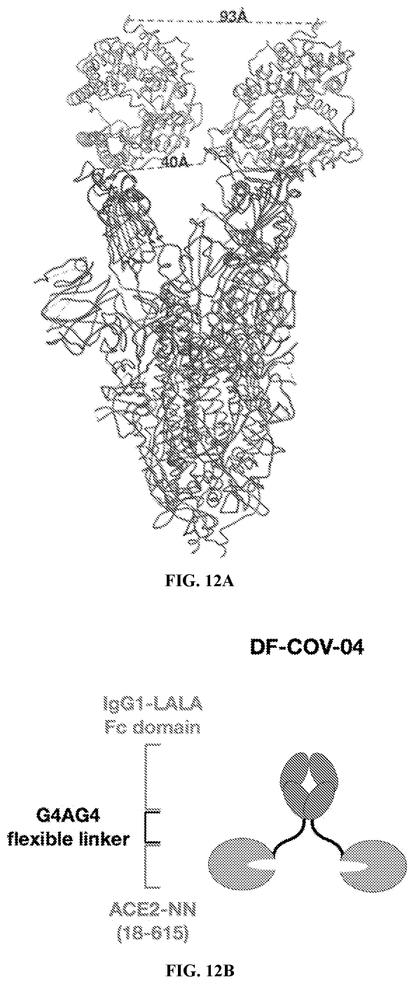

As additional constructs, DF-COV-03 and DF-COV-04 were designed and expressed. Their sequences are provided in Table 7. As exemplary depictions of various constructs, FIG. 10A to FIG. 12B are provided, in which FIG. 10A depicts DF-COV-01, FIG. 10B depicts DF-COV-02, FIG. 11 depicts DF-COV-03, FIG. 12A depicts an overlay of full-length SARS-CoV-2 S-protein structure with ACE2 metalloprotease domain binding to the SARS-CoV-2 S-protein RBD, and FIG. 12B depicts DF-COV-04.

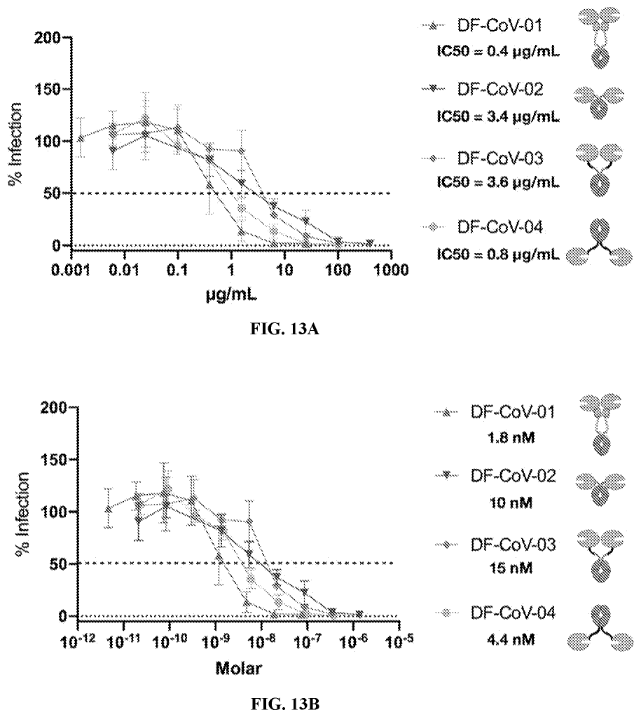

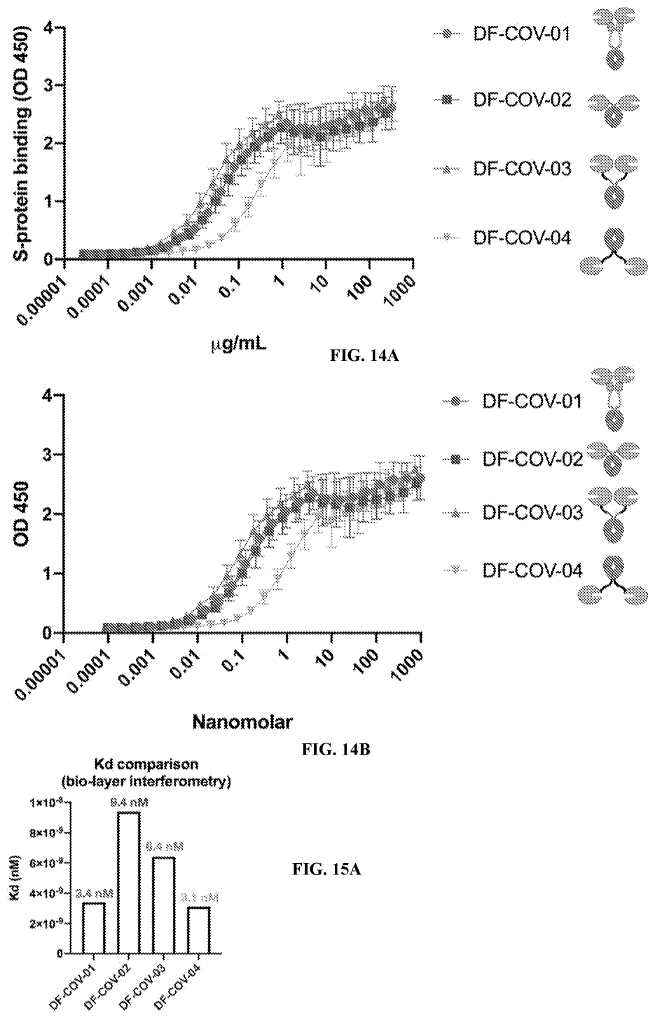

Surprisingly, the results showed that reversing the orientation of the ACE2 and Fc domain, as in DF-COV-04, improves neutralization (FIG. 13). Again surprisingly, the results showed that the relative potency of in vitro viral neutralization in FIG. 13 (and FIG. 6) does not correlate with binding avidity to the viral S-protein (FIG. 14). Both DF-COV-01 and DF-COV-02 have identical avidity for viral S-protein, but DF-COV-01 neutralizes more potently than DF-COV-02 in vitro. DF-COV-03 has a higher binding avidity for the viral S-protein than DF-COV-01, but neutralizes less potently. Conversely, DF-COV-04 neutralizes more potently than DF-COV-02 and DF-COV-03 but has a lower binding avidity.

### Example 14: Binding Affinities, Stabilities, and Functions of the Constructs

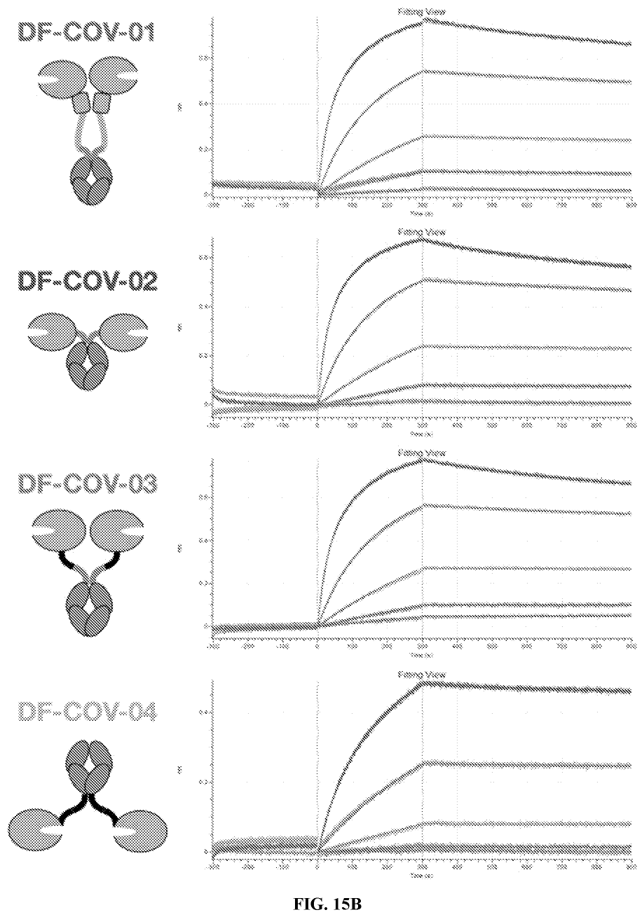

The binding affinity of DF-COV-01, DF-COV-02, DF-COV-03, and DF-COV-04 to the SARS-CoV-2 S-protein was determined by bio-layer interferometry (Octet) with DF-COV compounds immobilized and SARS-CoV-2 S-protein in solution. DF-COV-01 and DF-COV-02 had a higher affinity for the SARS-CoV-2 S-protein than DF-COV-03 and DF-COV-04. (FIG. 15A and FIG. 15B).

Based on the results, in some embodiments, DF-COV-01 is a preferred compound, at least due to the unexpected findings that it (1) has a higher avidity for the viral S-protein than compounds 2 and 3 (as measured in FIG. 15A and FIG. 15B), (2) has a lower IC50 in pseudovirus neutralization studies than the other compounds, (3) has a longer serum half-life in hamster than the other compounds, and (4) in the hamster model it lowers oropharyngeal, nasal turbinate, and lung viral titers more than other compounds and protects against weight loss more so than other compounds.

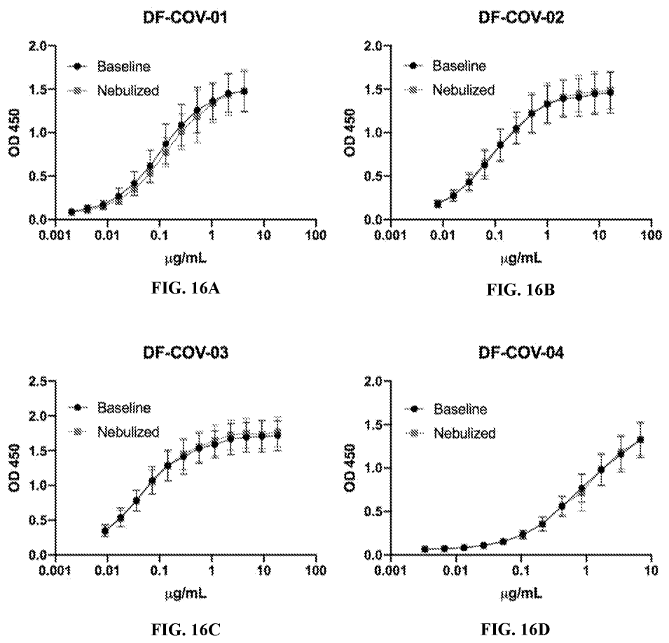

In addition, each of DF-COV-01, DF-COV-02, DF-COV-03, and DF-COV-04 was nebulized with a vibrating mesh nebulizer and then collected in a microcentrifuge tube. The binding of nebulized compound to immobilized SARS-CoV-2 S-protein was compared with that of a sample taken prior to nebulization. Binding was detected via an anti-human IgG-HRP secondary antibody. For all four DF-COV compounds there was no significant difference in S-protein binding avidity between nebulized sample and non-nebulized sample. (FIG. 16A to FIG. 16D).

Based on the results, in some embodiments, the route of administration is either IV or via inhalation (nebulization). FIG. 16A to FIG. 16D demonstrate that the compounds are stable when nebulized (no decrease in avidity for the viral S-protein after nebulization). It is also believed that the compounds are capable of neutralizing pseudovirus after they are nebulized.

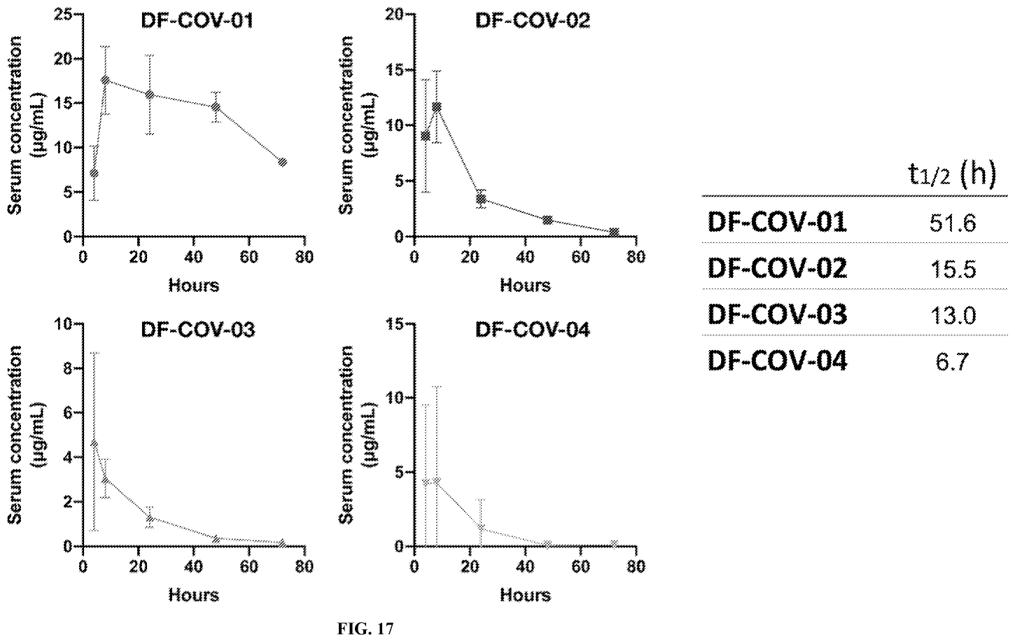

FIG. 17 demonstrates the unexpected finding that DF-COV-01, an ACE2-Fc fusion containing both the peptidase domain (PD) and the collectrin-like domain (CLD) of ACE2, has a longer half-life than DF-COV-02, an otherwise identical ACE2-Fc fusion in which the CLD is omitted. DF-COV-03, which contains an artificial flexible linker separating the ACE2 PD from the Fc domain in place of the CLD, also has a shorter half-life than the CLD-containing DF-COV-01. DF-COV-04, in which the ACE2 PD is appended to the C-terminus of the Fc domain and separated by an artificial flexible linker (and with no ACE2 CLD included), also had a short half-life.

DF-COV-01 is an ACE2-Fc fusion that includes the juxtamembrane (extracellular) portion of the ACE2 CLD (amino acids 616 through 740 of ACE2). It was unexpectedly, determined that DF-COV-01 has a much longer serum half-life than DF-COV-02, which is an otherwise identical ACE2-Fc fusion in which the CLD is omitted. DF-COV-01 also had a much longer serum half-life than DF-COV-03, an ACE2-Fc fusion in which an artificial flexible linker was substituted in place of the CLD, and DF-COV-04, an ACE2-Fc fusion in which the orientation of ACE2 and the Fc domain was reversed and in which the CLD was also omitted (see working examples below). Additionally, in a head-to-head comparison of DF-COV-01, -02, -03, and -04, DF-COV-01 was the only ACE2-Fc fusion that demonstrated in vivo efficacy against SARS-CoV-2 infection by reducing lung viral titers and ameliorating weight loss due to infection.

As indicated above, differences between the serum half-life of DF-COV compounds were unexpected. Without being bound by theory, it is believed that the presence of the CLD promotes dimerization of the two ACE2 domains in DF-COV-01 and association of the ACE2 domains with one another distances them from the Fc domain, thereby reducing steric hinderance of its binding to Fc neonatal receptor (FcRn). The FcRn extends the half-life of Fc domain containing molecules by binding and chaperoning them away from the lysosome in endothelial cells and other cells within the body, where pinocytosed proteins are hydrolyzed. This chaperoning activity is also believed to enable enables efficient transcytosis of FcRn-containing compounds across endothelial and epithelial cell layers.

A published structure of full-length ACE2 obtained by cryogenic electron microscopy (e.g., PDB structures 6M18 and 6M1D) demonstrates that full-length ACE2 exists as a dimer (Yan et al. (2020) Science 367:1444-1448). By contrast, previous structures of the ACE2 peptidase domain (PD) alone did not detect dimerization (e.g., PDB structures 1R42, 1R4L, and 2AJF, each of which lacks the CLD, of Towler et al. (2004) J. Biol. Chem. 279:17996-18007). The dimerzation of full-length ACE2 was found to be mediated by a portion of the juxtamembrane CLD that was termed the “neck” domain in the study (amino acids 616-726). In a subset of molecular species, a portion of the PD was also found to mediate direct dimerization, but the absence of dimerization in studies with purified ACE2 PD would indicate that the presence of the CLD is necessary for dimerization.

This structure of full-length ACE2 indicates that the two ACE2 domains of DF-COV-01, which can be thought of as flexible arms, are believed to associate with one another to some degree and form a “dimer” such that the association of the two ACE2 domains of DF-COV-01 with one another may limit their proximity to, and thus limit their steric hindrance of, the DF-COV-01 Fc domain. The absence of this steric hindrance is believed to facilitate FcRn binding.

Consistently, DF-COV-02, which is an otherwise identical ACE2-Fc that lacks the CLD, was found to have a much shorter serum half-life than DF-COV-01. Since the ACE2 domains of DF-COV-02 lack the CLD, they would not be expected to associate with one another and would be free to weakly associate with or occupy space in the proximity of the Fc domain of DF-COV-02, which is believed to sterically hinder FcRn binding.

Moreover, DF-COV-03 also had a much shorter serum half-life than DF-COV-01. Like DF-COV-02, the CLD is omitted in DF-COV-03. Unlike DF-COV-02, however, an artificial flexible linker is inserted in its place, between the ACE2 PD and the hinge domain of the Fc domain. A more flexible IgG1 hinge domain is also utilized in DF-COV-03. Together, this results in a long and flexible amino acid sequence separating the ACE2 PD from the Fc domain. The fact that a long and flexible sequence separating the PD from the Fc domain is insufficient to impart the longer half-life exhibited by DF-COV-01, which contains the CLD in this location, emphasizes the importance of the sequence identity of the CLD in governing the serum half-life of an ACE2-Fc, which is believed to be due to the propensity of CLD-containing ACE2 domains to associate with one another, limiting their proximity to and steric hindrance of the Fc domain. Although the ACE2 domains of DF-COV-03 are allowed flexibility to orient with respect to the Fc domain, they are still free to weakly associate with or occupy space in the proximity of the Fc domain and thus sterically hinder FcRn binding.

DF-COV-04, in which the ACE2 PD is appended to the C-terminus of the Fc domain and separated by an artificial flexible linker (and with no ACE2 CLD included), also had a short half-life.

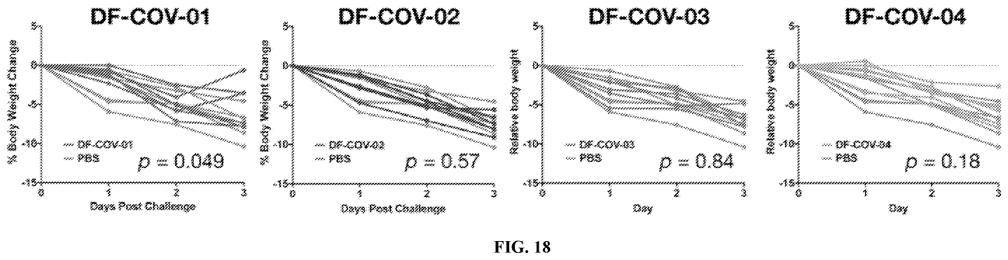

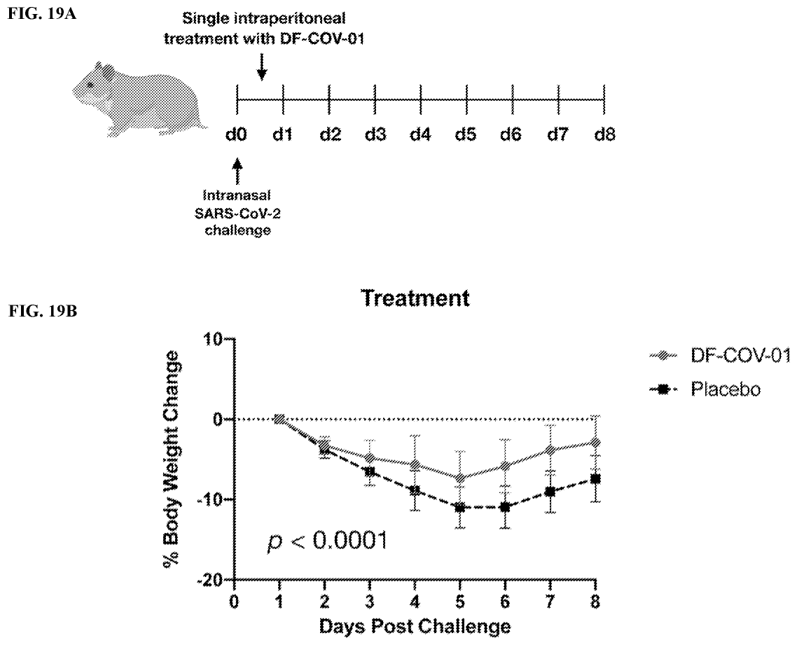

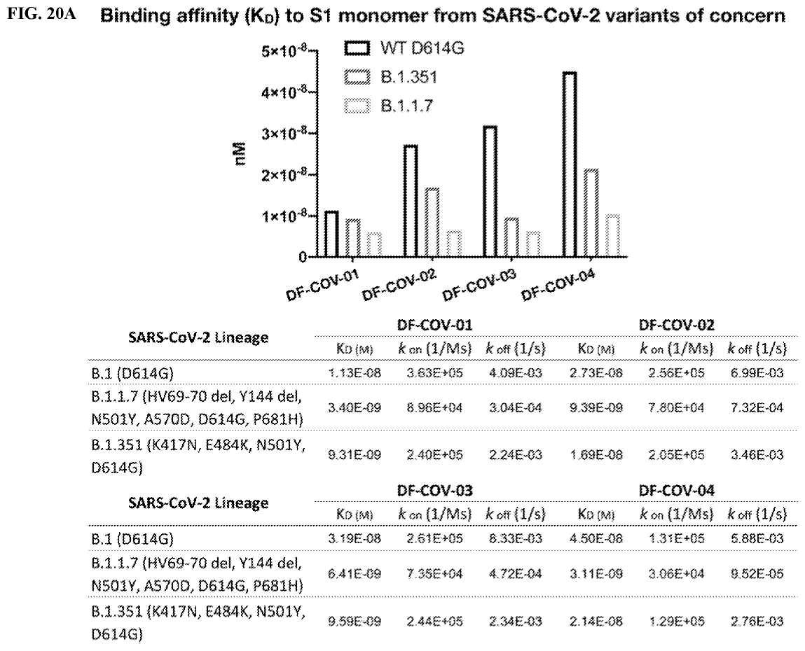

FIG. 18 demonstrates that DF-COV-01 was the only compound that limited body weight loss, a metric of disease severity, as demonstrated by a daily weight trend that diverged from the vehicle control group and demonstrated recovery of body weight. A mixed-effects model with the Geisser-Greenhouse correction and Šidák's multiple comparisons test in the Prism v9 software package was utilized. This additional analysis of data from the experiment presented in FIG. 9 and discussed above at least in example 12 further demonstrates that DF-COV-01, an ACE2-Fc fusion containing the CLD of ACE2, was the only ACE2-Fc fusion capable of ameliorating disease severity in SARS-CoV-2 treated hamsters. DF-COV-02, an otherwise identical ACE2-Fc fusion in which the CLD is omitted, was not capable of attenuating disease severity. DF-COV-03, which contains an artificial flexible linker separating the ACE2 peptidase domain (PD) from the Fc domain in place of the CLD, was also not capable of attenuating disease severity. DF-COV-04, in which the ACE2 PD is appended to the C-terminus of the Fc domain and separated by an artificial flexible linker (and with no ACE2 CLD included), was also not capable of attenuating disease severity despite having an equivalent binding affinity for the SARS-CoV-2 S-protein trimer as DF-COV-01 (see FIG. 15A). This data suggests that DF-COV-01 has superior anti-viral activity compared with the other DF-COV compounds when administered systemically. If administered by a different route (e.g., via inhalation) or systemically at a higher dose, the other DF-COV compounds may demonstrate anti-viral activity.

FIG. 19A and FIG. 19B demonstrate the ability of DF-COV-01 to treat hamsters in a therapeutic model of SARS-CoV-2 infection, in which hamsters are treated twelve hours after being challenged with 1×10⁴ PFU of SARS-CoV-2. Half of the hamsters (20) were treated with a single dose of 150 mg/kg of DF-COV-01 administered by intraperitoneal injection and the other half (20) were treated with an intraperitoneal injection of placebo (PBS). Daily weights were recorded. Treatment with DF-COV-01 resulted in a highly statistically significant reduction in weight loss compared with placebo, indicating that it ameliorated the severity of disease. A mixed-effects model with the Geisser-Greenhouse correction and Šidák's multiple comparisons test was utilized (Prism v9 software package). Thus, DF-COV-01 is demonstrated to treat disease rather than provide prophylaxis against infection. DF-COV-01 reduced weight loss from SARS-CoV-2 infection, indicating that it ameliorated the severity of disease.

DF-COV-01 was the only compound to demonstrate in vivo activity against SARS-CoV-2 in an experiment in which separate groups of hamsters received doses of equivalent mass of DF-COV-01, DF-COV-02, DF-COV-03, or DF-COV-04 each day for three days. The fact that DF-COV-04 did not demonstrate activity in this model, despite having a similar high binding affinity to the SARS-CoV-2 S-protein as DF-COV-01 and a similar, albeit slightly higher IC50 in neutralization studies, suggests that the superiority of DF-COV-01 over the other DF-COV compounds is believed to be due to a factor other than its high binding affinity and neutralization potency alone, possibly drug exposure from its higher half-life, or some other factor.

### Example 15: Assessing Binding to Viral Variants

A pseudovirus is generated using a replication-deficient HIV-1 backbone that contains a mutation to prevent HIV envelope glycoprotein expression and a luciferase gene to direct luciferase expression in target cells. HEK293-T cells are co-transfected with two plasmids: one an expression vector for the pseudovirus spike protein (from the viral variant) and the other bearing the Env-defective HIV-1 genome. Supernatant containing virus particles are harvested 48 hours after transfection, are concentrated using Centricon 70 concentrators, and are stored frozen at −80° C. Target cells are generated by transiently transfecting 293T cells with a human ACE2 expression vector (pcDNA-hACE2). ACE2-transfected 293T cells are cells used as target cells 24 hours after transfection. A titration of pseudovirus is performed on 293T cells transiently transfected with human ACE2 receptor to determine the volume of virus need to generate 50,000 counts per second (cps) in the infection assay. The appropriate volume of pseudovirus is pre-incubated with serial dilutions of DF-COV-01, DF-COV-02, or an irrelevant Fc fusion for 1 hour at room temperature before adding to 293T cells expressing human ACE2. Media containing pseudovirus is replaced by the fresh complete media 24 hours later. After additional 24 hours, the infection is quantified by luciferase detection with BrightGlo luciferase assay (Promega) and read in a Victor3 plate reader (Perkin Elmer).

For binding experiments conducted and described herein, binding to recombinant S1 proteins from SARS-CoV-02 variants was assessed using the Octet® RED384 biolayer interferometry system. DF-COV-01, DF-COV-02, DF-COV-03, or DF-COV-04 was immobilized to anti-human IgG Fc Capture (AHC) biosensors (Sartorius) following the protocol recommended by the manufacturer. The S1 proteins (Sino Biological) were diluted using the running buffer (PBS, 0.02% Tween 20, 2 mg/ml BSA) to 1.23-100 nM and transferred to the 96-well plate. Sensors immobilized with the DF-COV ACE2-Fc compounds were dipped in the wells containing the diluted S1 proteins for 5 minutes to measure association and then dipped in running buffer for 10 minutes to measure dissociation. Signal from sensors without DF-COV compound dipped in the S1 proteins or running buffer were used as references. Kinetic analysis was performed using the Octet® Data Analysis HT Version 12.0 software and curves were fit using the 1:1 binding mode.

It has been demonstrated that escape of SARS-CoV-02 variants from neutralization by monoclonal antibodies and serum from vaccinated patients is due to a reduction or loss of binding of these antibodies to the receptor binding domain (RBD) of the virus as a result of mutations within the RBD (Planas et al. (2021) Nature doi.org/10.1038/s41586-021-03777-9). Thus, assessment of binding to recombinant S1 proteins, which include RBD mutations and other mutations within the S-protein in individual SARS-CoV-2 variants can be used to determine whether DF-COV compounds bind and would be expected to neutralize that SARS-CoV-2 variant. Using such approaches, binding was assessed for representative viral variants, including using recombinant S1 proteins from the Alpha (B.1.1.7) and Beta (B.1.351) coronaviruses. Similar binding assessments can be applicatey to a variety of other viral variants, including, without limitation, for example one that is resistant to neutralization by a monoclonal antibody capable of neutralizing other coronaviruses; is a variant of SARS-CoV-2 that is resistant to neutralization by a monoclonal antibody capable of neutralizing SARS-CoV-2; is resistant to the immunity imparted by a coronavirus vaccine; is a variant of SARS-CoV-2 that is resistant to the immunity imparted by a SARS-CoV-2 vaccine; is resistant to natural immunity imparted by prior coronavirus infection; is a variant of SARS-CoV-2 that is resistant to natural immunity imparted by prior SARS-CoV-2 infection; harbors an E484 substitution in the S-protein; harbors a N501 substitution in the S-protein; harbors a K417 substitution in the S-protein; harbors E484 and N501 substitutions in the S-protein; harbors an E484K substitution in the S-protein; harbors an E484Q substitution in the S-protein; harbors a N501Y substitution in the S-protein; coronavirus harbors a K417N substitution in the S-protein; harbors E484K and N501Y substitutions in the S-protein; harbors E484, N501, and K417 substitutions in the S-protein; harbors E484K, N501Y, and K417N substitutions in the S-protein; harbors an L452 substitution in the S-protein; harbors an L452R substitution in the S-protein; harbors a T478 substitution in the S-protein; harbors a T478K substitution in the S-protein; harbors an L452 and a T478 substitution in the S-protein; harbors an L452R and a T478K substitution in the S-protein; descends from the B.1.1.7 lineage, also known as 20I/501Y.V1, the “British variant,” or Alpha variant; is the B.1.1.7 lineage, also known as 20I/501Y.V1, or the “British variant,” or Alpha variant; descends from the B.1.351 lineage, also known as 20H/501Y.V2, the “South African COVID-19 variant,” or Beta variant (harbors E484K, N501Y, and K417N substitutions in addition to other substitutions outside of the S-protein receptor binding domain); is the B.1.351 lineage, also known as 20H/501Y.V2, the “South African COVID-19 variant,” or Beta variant (harbors E484K, N501Y, and K417N substitutions in addition to other substitutions outside of the S-protein receptor binding domain); descends from the B.1.1.248 lineage, also known as lineage P.1, the “Brazilian COVID-19 variant,” or Gamma variant (harbors E484K and N501Y substitutions in addition to other substitutions outside of the S-protein receptor binding domain); is the B.1.1.248 lineage, also known as linage P.1, the “Brazilian COVID-19 variant,” or Gamma variant (harbors E484K and N501Y substitutions in addition to other substitutions outside of the S-protein receptor binding domain); descends from the B.1.617 lineage; descends from the B.1.617.1 lineage (harbors E484Q substitution); is the B.1.617.1 lineage (harbors E484Q substitution); descends from the B.1.617.2 lineage, also known as Delta variant (harbors L452R and T478K substitutions in addition to other S-protein mutations inside and outside of the receptor binding domain); is the B.1.617.2 lineage, also known as Delta variant (harbors L452R and T478K substitutions in addition to other S-protein mutations inside and outside of the receptor binding domain); descends from the B.1.617.3 lineage (harbors E484Q substitution); and/or is the B.1.617.3 lineage (harbors E484Q substitution).

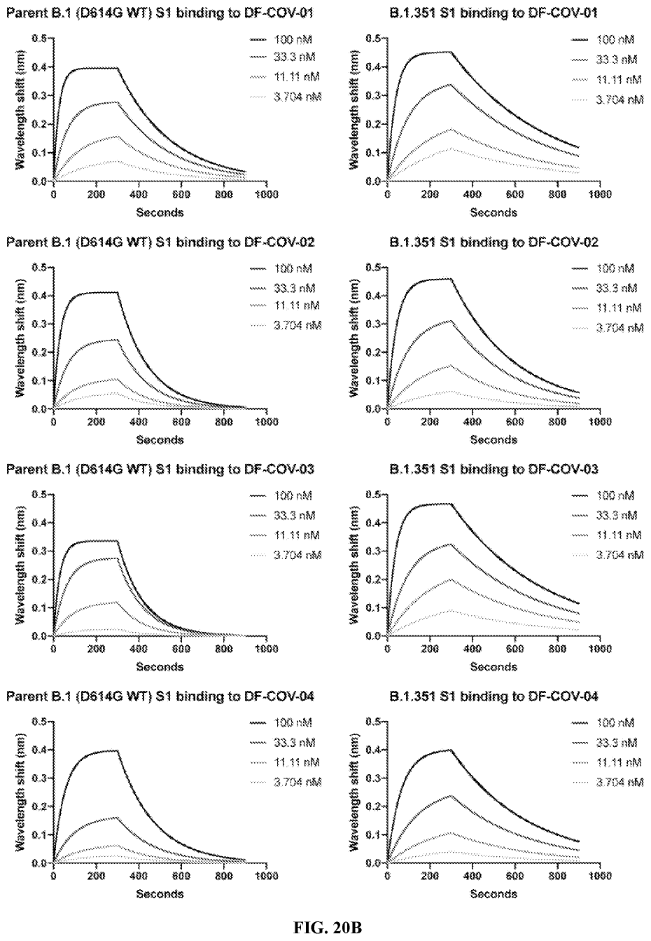

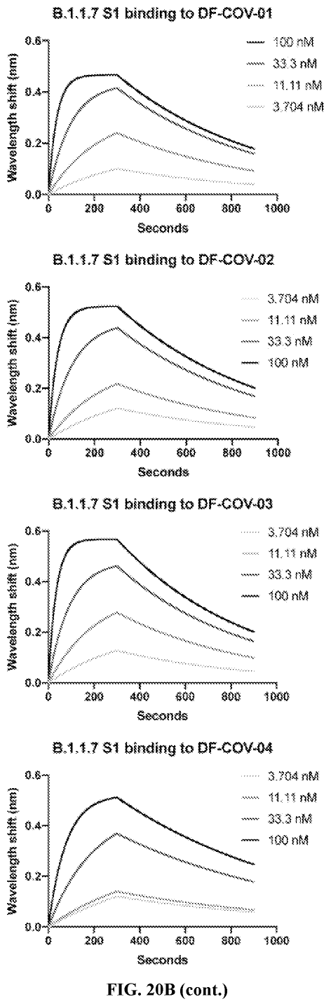

FIG. 20A and FIG. 20B demonstrate binding characteristics of DF-COV compounds to SARS-CoV-2 variants. The binding of immobilized DF-COV-01, DF-COV-02, DF-COV-03, and DF-COV-04 to soluble recombinant S1 proteins from the B.1.1.7 (alpha), B.1.351 (beta), and parent B.1 lineage (D614G) was assessed via biolayer interferometry (BLI). The B.1.351 lineage contains the E484K substitution, which is known to inhibit binding of several therapeutic monoclonal antibodies in published studies, including bamlanivimab (Ly-CoV555) which is unable to neutralize this variant in vitro. Similarly, REGN10933, one of the two antibodies in the REGN-COV2 cocktail, is known to demonstrate a marked reduction in neutralization potency against B.1.351 in published studies. The B.1.351 lineage is also known to exhibit vaccine resistance. All four DF-COV compounds bound avidly to S1 protein from both the B.1.1.7 and B.1.351 lineages. In fact, for each of the DF-COV compounds, the binding affinity for variant S1 protein was higher than for the parent B.1 S1 protein. These results are important because the Beta variant (B.1.351) is known to be resistant to several monoclonal antibodies and to have resistance to several vaccines, which is known to be driven by an amino acid substitutions at positions E484 and K417 in the receptor binding domain of the viral S-protein (Wang et al. (2021) Nature 593:130-135 describing B.1.351 monoclonal antibody resistance; Zhou et al. (2021) Cell 184:2348-2361 describing B.1.351 vaccine resistance). Other variants of concern that demonstrate resistance to monoclonal antibodies and serum from vaccinated patients, such as the P.1 variant, also have an amino acid substitutions at these positions (Wang et al. (2021) Cell Host Microbe 29:747-751).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An ACE2-Fc fusion polypeptide comprising an ACE2 extracellular domain polypeptide or fragment thereof, a hinge polypeptide, and a fragment crystallizable (Fc) domain or fragment thereof, wherein the ACE2 extracellular domain is enzymatically inactive, and wherein the Fc domain or fragment thereof has attenuated binding affinity for a Fcγ receptor.

2. The ACE2-Fc fusion polypeptide of claim 1, wherein the ACE2 extracellular domain polypeptide comprises an amino acid sequence having at least 90% identity to the amino acid sequence of any one of SEQ ID NOs: 1-4.

3. The ACE2-Fc fusion polypeptide of claim 1 or 2, wherein the ACE2 extracellular domain polypeptide comprises at least one amino acid substitution at a residue position selected from the group consisting of H374, H378, R273, H345, H345, H505, H505, R169, W271, and K481.

4. The ACE2-Fc fusion polypeptide of any one of claims 1-3, wherein the ACE2 extracellular domain polypeptide comprises at least one amino acid substitution selected from the group consisting of H374N, H378N, R273Q, H345A, H345L, H505A, H505L, R169Q, W271Q, and K481Q relative to a wild type ACE2 polypeptide.

5. The ACE2-Fc fusion polypeptide of 3 or 4, wherein the at least one amino acid substitution is a H374N or H378N substitution relative to a wild type ACE2 polypeptide.

6. The ACE2-Fc fusion polypeptide of 3 or 4, wherein the at least one amino acid substitution is a H374N and H378N substitution relative to a wild type ACE2 polypeptide.

7. The ACE2-Fc fusion polypeptide of claim 3 or 4, wherein the the at least one amino acid substitution is R273Q, H345A, H345L, H505A, H505L amino acid substitutions.

8. The ACE2-Fc fusion polypeptide of claim 3 or 4, wherein the at least one amino acid substitution is R169Q, W271Q, and K481Q amino acid substitutions.

9. The ACE2-Fc fusion polypeptide of any one of claims 1-8, wherein the ACE2 extracellular domain has affinity for a coronavirus.

10. The ACE2-Fc fusion polypeptide of claim 9, wherein the coronavirus is selected from the group consisting of SARS-CoV-1 and the SARS-CoV-2.

11. The ACE2-Fc fusion polypeptide of any one of claims 1-10, wherein the Fc domain comprises an amino acid sequence comprising at least one amino acid substitution relative to a wild-type Fc domain that decreases or eliminates binding of the Fc domain to a Fc receptor.

12. The ACE2-Fc fusion polypeptide of claim 11, wherein the Fc receptor is a FcγIIa receptor.

13. The ACE2-Fc fusion polypeptide of any one of claims 1-11, wherein the Fc domain comprises an amino acid sequence from Table 5.

14. The ACE2-Fc fusion polypeptide of claim 11, wherein the Fc domain is derived from an IgG4 antibody.

15. The ACE2-Fc fusion polypeptide of claim 11, wherein the Fc domain comprises at least one amino acid substitution at a residue position selected from the group consisting of L235, and P329.

16. The ACE2-Fc fusion polypeptide of claim 14, wherein the ACE2-Fc fusion polypeptide comprises an S228P or L235E amino acid substitution.

17. The ACE2-Fc fusion polypeptide of claim 14, wherein the ACE2-Fc fusion polypeptide comprises an S228P and L235E amino acid substitution.

18. The ACE2-Fc fusion polypeptide of claim 14, wherein the Fc domain comprises an amino acid sequence of any one of SEQ ID NOs: 33-36.

19. The The ACE2-Fc fusion polypeptide of claim 11, wherein the Fc domain is derived from an IgG1 antibody.

20. The ACE2-Fc fusion polypeptide of claim 19, wherein the Fc domain comprises at least one amino acid substitution selected from the group consisting of L234A, L235A, N297A, N297D, and P329G.

21. The ACE2-Fc fusion polypeptide of claim 19, wherein the Fc domain comprises an amino acid sequence have at least 90% sequence identity to any one of SEQ ID NOs: 37-42 or SEQ ID NO: 55.

22. The ACE2-Fc fusion polypeptide of claim 11 wherein the Fc domain is derived from an IgG2 antibody.

23. The ACE2-Fc fusion polypeptide of claim 22 wherein the Fc domain comprises the amino acid sequence of SEQ ID NO: 44.

24. The ACE2-Fc fusion polypeptide of any one of claims 1-23, wherein the hinge region comprises an amino acid sequence from Table 2, 3, or 4.

25. The ACE2-Fc fusion polypeptide of any one of claims 1-23, wherein the hinge region consists of a proline or a cysteine-proline dipeptide.

26. An ACE2-Fc fusion polypeptide comprising an amino acid sequence having at least 90% identity to SEQ ID NO: 48, 49, 56, or 57.

27. A nucleic acid molecule encoding the ACE2-Fc fusion polypeptide of any one of claims 1-26.

28. A nucleic acid comprising a nucleotide sequence having at least 90% identity to SEQ ID NO: 50, 51, 58, or 59.

29. A vector comprising the nucleic acid molecule of claim 27 or 28.

30. The vector of claim 29, wherein the vector is an expression vector.

31. A cell comprising the vector of claim 29 or 30.

32. The cell of claim 31, wherein the cell is mammalian cell.

33. A method of sequestering a coronavirus comprising contacting a fluid comprising a coronavirus with the ACE2-Fc fusion polypeptide of any one of claims 1-26, wherein the ACE2-Fc fusion polypeptide binds the coronavirus, thereby sequestering the coronavirus.

34. The method of claim 33, wherein the sequestered coronavirus is incapable of binding to a full length ACE2 polypeptide.

35. A method of inhibiting a coronavirus from binding to an endogenous ACE2 polypeptide expressed by a cell, the method comprising contacting a fluid in communication with the cell with the ACE2-Fc fusion polypeptide of any one of claims 1-26 wherein the ACE2-Fc fusion polypeptide binds the coronavirus, thereby inhibiting the coronavirus from binding endogenously expressed ACE2 polypeptides.

36. The method of any one of claims 33 to 35, wherein the fluid is an interstitial fluid, blood, plasma, serum, mucous, cerebrospinal fluid, or lymph.

37. A method for treating a subject having or suspected of having a coronavirus infection, the method comprising administering a therapeutically effective amount of a pharmaceutical composition comprising the ACE2-Fc fusion polypeptide of any one of claims 1-26 to the subject.

38. A method for preventing a coronavirus infection in a subject at risk of infection, the method comprising administering an effective amount of a pharmaceutical composition comprising the ACE2-Fc fusion polypeptide of any one of claims 1-26 to the subject.

39. A method of treating or preventing antibody dependent enhancement of a coronavirus infection in a subject, the method comprising administering to the subject a therapeutically effective amount of a pharmaceutical composition comprising the ACE2-Fc fusion polypeptide of any one of claims 1-26 to the subject.

40. The method of any one of claims 33-39, wherein the coronavirus is selected from the group consisting of SARS-CoV-1 and SARS-CoV-2.

41. The method of any one of claims 33-36 wherein the cell is a mammalian cell.

42. The method of claim 41, wherein the mammalian cell is a human cell.

43. The method of any one of claims 37-39, wherein the subject is a mammal.

44. The method of claim 43, wherein the subject is a human.

45. The method of any one of claims 37-39, wherein the administering is selected from the group consisting of subcutaneous, intravenous, parenteral, intraperitoneal, intrathecal, oral, inhalation, nebulization, and transdermal.

46. The method of any one of claims 33-39, wherein the
a. coronavirus is resistant to neutralization by a monoclonal antibody capable of neutralizing other coronaviruses;
b. coronavirus is a variant of SARS-CoV-2 that is resistant to neutralization by a monoclonal antibody capable of neutralizing SARS-CoV-2;
c. coronavirus is resistant to the immunity imparted by a coronavirus vaccine;
d. coronavirus is a variant of SARS-CoV-2 that is resistant to the immunity imparted by a SARS-CoV-2 vaccine;
e. coronavirus is resistant to natural immunity imparted by prior coronavirus infection;
f. coronavirus is a variant of SARS-CoV-2 that is resistant to natural immunity imparted by prior SARS-CoV-2 infection;
g. coronavirus harbors an E484 substitution in the S-protein;
h. coronavirus harbors a N501 substitution in the S-protein;
i. coronavirus harbors a K417 substitution in the S-protein;
j. coronavirus harbors E484 and N501 substitutions in the S-protein;
k. coronavirus harbors an E484K substitution in the S-protein;
l. coronavirus harbors an E484Q substitution in the S-protein;
m. coronavirus harbors a N501Y substitution in the S-protein;
n. coronavirus harbors a K417N substitution in the S-protein;
o. coronavirus harbors E484K and N501Y substitutions in the S-protein;
p. coronavirus harbors E484, N501, and K417 substitutions in the S-protein;
q. coronavirus harbors E484K, N501Y, and K417N substitutions in the S-protein;
r. coronavirus harbors an L452 substitution in the S-protein;
s. coronavirus harbors an L452R substitution in the S-protein;
t. coronavirus harbors a T478 substitution in the S-protein;
u. coronavirus harbors a T478K substitution in the S-protein;
v. coronavirus harbors an L452 and a T478 substitution in the S-protein;
w. coronavirus harbors an L452R and a T478K substitution in the S-protein;
x. coronavirus descends from the B.1.1.7 lineage, also known as 20I/501Y.V1, the “British variant,” or Alpha variant;
y. coronavirus is the B.1.1.7 lineage, also known as 20I/501Y.V1, the “British variant,” or Alpha variant;
z. coronavirus descends from the B.1.351 lineage, also known as 20H/501Y.V2, the “South African COVID-19 variant,” or Beta variant (harbors E484K, N501Y, and K417N substitutions in addition to other substitutions outside of the S-protein receptor binding domain);
aa. coronavirus is the B.1.351 lineage, also known as 20H/501Y.V2, the “South African COVID-19 variant,” or Beta variant (harbors E484K, N501Y, and K417N substitutions in addition to other substitutions outside of the S-protein receptor binding domain);
bb. coronavirus descends from the B.1.1.248 lineage, also known as the “Brazilian COVID-19 variant,” lineage P.1, or Gamma variant (harbors E484K and N501Y substitutions in addition to other substitutions outside of the S-protein receptor binding domain);
cc. coronavirus is the B.1.1.248 lineage, also known as the “Brazilian COVID-19 variant,” lineage P.1, or Gamma variant (harbors E484K and N501Y substitutions in addition to other substitutions outside of the S-protein receptor binding domain);
dd. coronavirus descends from the B.1.617 lineage;
ee. coronavirus descends from the B.1.617.1 lineage (harbors an E484Q substitution);
ff. coronavirus is the B.1.617.1 lineage (harbors an E484Q substitution);
gg. coronavirus descends from the B.1.617.2 lineage, also known as Delta variant (harbors L452R and T478K substitutions in addition to other S-protein mutations);
hh. coronavirus is the B.1.617.2 lineage, also known as Delta variant (harbors L452R and T478K substitutions in addition to other S-protein mutations);
ii. coronavirus descends from the B.1.617.3 lineage (harbors E484Q substitution); and/or
jj. coronavirus is the B.1.617.3 lineage (harbors E484Q substitution).


SEQUENCE LISTING (62 sequences)

SEQ ID NO: 1 - protein, 723 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 2 - protein, 691 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSR

SEQ ID NO: 3 - protein, 598 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - protein, 595 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSP

SEQ ID NO: 5 - protein, 13 aa, Artificial Sequence
  source: /note=“Description of Artificial Sequence Synthetic peptide”
VESKYGPPCPPCP

SEQ ID NO: 6 - protein, 12 aa, Artificial Sequence
  source: /note=“Description of Artificial Sequence Synthetic peptide”
ESKYGPPCPPCP

SEQ ID NO: 7 - protein, 11 aa, Artificial Sequence
  source: /note=“Description of Artificial Sequence Synthetic peptide”
SKYGPPCPPCP

SEQ ID NO: 8 - protein, 10 aa, Artificial Sequence
  source: /note=“Description of Artificial Sequence Synthetic peptide”
KYGPPCPPCP

SEQ ID NO: 9 - protein, 9 aa, Artificial Sequence
  source: /note=“Description of Artificial Sequence Synthetic peptide”
YGPPCPPCP

SEQ ID NO: 10 - protein, 8 aa, Artificial Sequence
  source: /note=“Description of Artificial Sequence Synthetic peptide”
GPPCPPCP

SEQ ID NO: 11 - protein, 7 aa, Artificial Sequence
  source: /note=“Description of Artificial Sequence Synthetic peptide”
PPCPPCP

SEQ ID NO: 12 - protein, 6 aa, Artificial Sequence
  source: /note=“Description of Artificial Sequence Synthetic peptide”
PCPPCP

SEQ ID NO: 13 - protein, 5 aa, Artificial Sequence
  source: /note=“Description of Artificial Sequence Synthetic peptide”
CPPCP

SEQ ID NO: 14 - protein, 4 aa, Artificial Sequence
  source: /note=“Description of Artificial Sequence Synthetic peptide”
PPCP

SEQ ID NO: 15 - protein, 3 aa, Artificial Sequence
  source: /note=“Description of Artificial Sequence Synthetic peptide”
PCP

SEQ ID NO: 16 - protein, 12 aa, Artificial Sequence
  source: /note=“Description of Artificial Sequence Synthetic peptide”
EPKSCDKTHTCP

SEQ ID NO: 17 - protein, 11 aa, Artificial Sequence
  source: /note=“Description of Artificial Sequence Synthetic peptide”
PKSCDKTHTCP

SEQ ID NO: 18 - protein, 10 aa, Artificial Sequence
  source: /note=“Description of Artificial Sequence Synthetic peptide”
KSCDKTHTCP

SEQ ID NO: 19 - protein, 9 aa, Artificial Sequence
  source: /note=“Description of Artificial Sequence Synthetic peptide”
SCDKTHTCP

SEQ ID NO: 20 - protein, 8 aa, Artificial Sequence
  source: /note=“Description of Artificial Sequence Synthetic peptide”
CDKTHTCP

SEQ ID NO: 21 - protein, 7 aa, Artificial Sequence
  source: /note=“Description of Artificial Sequence Synthetic peptide”
DKTHTCP

SEQ ID NO: 22 - protein, 6 aa, Artificial Sequence
  source: /note=“Description of Artificial Sequence Synthetic peptide”
KTHTCP

SEQ ID NO: 23 - protein, 5 aa, Artificial Sequence
  source: /note=“Description of Artificial Sequence Synthetic peptide”
THTCP

SEQ ID NO: 24 - protein, 4 aa, Artificial Sequence
  source: /note=“Description of Artificial Sequence Synthetic peptide”
HTCP

SEQ ID NO: 25 - protein, 3 aa, Artificial Sequence
  source: /note=“Description of Artificial Sequence Synthetic peptide”
TCP

SEQ ID NO: 26 - protein, 12 aa, Artificial Sequence
  source: /note=“Description of Artificial Sequence Synthetic peptide”
ERKCCVECPPCP

SEQ ID NO: 27 - protein, 11 aa, Artificial Sequence
  source: /note=“Description of Artificial Sequence Synthetic peptide”
RKCCVECPPCP

SEQ ID NO: 28 - protein, 10 aa, Artificial Sequence
  source: /note=“Description of Artificial Sequence Synthetic peptide”
KCCVECPPCP

SEQ ID NO: 29 - protein, 9 aa, Artificial Sequence
  source: /note=“Description of Artificial Sequence Synthetic peptide”
CCVECPPCP

SEQ ID NO: 30 - protein, 8 aa, Artificial Sequence
  source: /note=“Description of Artificial Sequence Synthetic peptide”
CVECPPCP

SEQ ID NO: 31 - protein, 7 aa, Artificial Sequence
  source: /note=“Description of Artificial Sequence Synthetic peptide”
VECPPCP

SEQ ID NO: 32 - protein, 6 aa, Artificial Sequence
  source: /note=“Description of Artificial Sequence Synthetic peptide”
ECPPCP

SEQ ID NO: 33 - protein, 217 aa, Artificial Sequence
  source: /note=“Description of Artificial Sequence Synthetic polypeptide”
APEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 217 aa, Artificial Sequence
  source: /note=“Description of Artificial Sequence Synthetic polypeptide”
APEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 217 aa, Artificial Sequence
  source: /note=“Description of Artificial Sequence Synthetic polypeptide”
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 217 aa, Artificial Sequence
  source: /note=“Description of Artificial Sequence Synthetic polypeptide”
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLASSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 220 aa, Artificial Sequence
  source: /note=“Description of Artificial Sequence Synthetic polypeptide”
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 220 aa, Artificial Sequence
  source: /note=“Description of Artificial Sequence Synthetic polypeptide”
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 220 aa, Artificial Sequence
  source: /note=“Description of Artificial Sequence Synthetic polypeptide”
PCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 220 aa, Artificial Sequence
  source: /note=“Description of Artificial Sequence Synthetic polypeptide”
PCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 220 aa, Artificial Sequence
  source: /note=“Description of Artificial Sequence Synthetic polypeptide”
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 220 aa, Artificial Sequence
  source: /note=“Description of Artificial Sequence Synthetic polypeptide”
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 216 aa, Homo sapiens
APPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - protein, 330 aa, Homo sapiens [sequence omitted]

SEQ ID NO: 46 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 47 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 948 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 825 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPGESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - DNA, 2904 bp, Artificial Sequence
  source: /note=“Description of Artificial Sequence Synthetic polynucleotide”
atggaatggagctgggtctttctcttcttcctgtcagtaacgactggtgtccactcccagtcaacaatcgaggagcaggcaaagaccttcctggacaagtttaaccacgaagcggaggacctgttctaccagagcagcctggcgagctggaattacaacaccaacatcaccgaggagaacgtgcagaacatgaataacgctggcgacaagtggagcgccttcttgaaggagcaatccaccctggcccagatgtacccgctgcaagagatacagaacctgactgtgaagctgcaactgcaggccctgcagcagaacggctccagcgtgctgagcgaagacaagagcaaaaggctgaataccatattgaacacgatgagcaccatctacagcaccggcaaagtatgcaaccccgacaacccccaagagtgtctgttgttggaacccggcctgaacgagattatggcgaactccctggactacaacgaacgcctgtgggcatgggagtcttggaggtcagaggttggcaagcaactgaggccgttgtacgaagagtacgtggtgctgaagaacgaaatggcacgggccaatcattatgaggactacggtgattattggaggggcgactacgaggtgaacggcgtggacggctacgactatagcaggggccaacttatagaggacgtagagcacacttttgaggaaatcaaacccctgtacgagcacttgcatgcctacgtacgcgccaaactgatgaatgcgtaccccagctacatcagccccatcggctgcctgcccgcacacctgctcggcgacatgtggggccgattctggaccaatctgtatagcctcaccgtgccctttggccagaagccgaacatagatgtgacggatgctatggtagaccaggcgtgggatgcacagcgcatcttcaaggaggccgagaagttcttcgtgagcgtcggcttgcccaacatgacccagggtttctgggagaactcaatgctgactgaccccggcaacgtacagaaagccgtatgccacccaactgcttgggacctgggtaagggagacttccggatcctcatgtgtaccaaggtgaccatggatgactttctcaccgcccacaatgaaatgggcaacatccagtacgatatggcctacgcagcgcagccattccttctgaggaacggtgccaacgagggcttccatgaggcagtcggagagatcatgagcctgtcagcggcgacccctaaacatctgaagagcatcgggctgctgtctccggactttcaagaggacaatgagactgagatcaacttcctcctgaaacaggcgctgaccattgtaggcaccttgcccttcacctacatgctggagaagtggcgatggatggtgtttaagggcgagatcccgaaggaccagtggatgaaaaagtggtgggagatgaagagggagatcgtcggcgtagttgagccggtcccccacgacgaaacctactgcgatcctgccagcctgttccacgtgagcaacgattacagttttatcaggtactacaccaggaccctttaccaatttcagttccaggaggcactgtgccaggccgccaagcacgaaggccccctgcacaagtgcgacatcagcaacagcaccgaggcgggtcaaaagctgtttaacatgctgaggctgggcaagagcgagccgtggaccctggccctggaaaacgttgtgggcgcaaaaaacatgaacgtgaggcccctgctgaactacttcgagcccctgttcacctggctgaaggaccagaacaagaacagtttcgtgggctggagtaccgattggagtccctatgccgaccagagcatcaaggtgaggattagcctcaagagcgccctgggcgacaaggcctatgaatggaacgacaacgagatgtacctgttcaggtcaagcgtggcctacgccatgaggcagtacttcctgaaagtgaagaaccagatgatactgttcggcgaggaggacgtgagggtggccaacctgaagcccaggatatcattcaatttcttcgtgaccgctcccaagaacgtgagcgacatcatccccaggaccgaggtggagaaggccatcaggatgagccgcagcaggattaacgacgccttcaggctgaacgataacagcctggagttccttggcatccagccaaccctgggaccacccaaccagcctcccgttagcggacccccctgtcctccttgccctgctcctgaatttgagggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggcaagtaa

SEQ ID NO: 51 - DNA, 2535 bp, Artificial Sequence
  source: /note=“Description of Artificial Sequence Synthetic polynucleotide”
atggaatggagctgggtctttctcttcttcctgtcagtaacgactggtgtccactcccagtcaacaatcgaggagcaggcaaagaccttcctggacaagtttaaccacgaagcggaggacctgttctaccagagcagcctggcgagctggaattacaacaccaacatcaccgaggagaacgtgcagaacatgaataacgctggcgacaagtggagcgccttcttgaaggagcaatccaccctggcccagatgtacccgctgcaagagatacagaacctgactgtgaagctgcaactgcaggccctgcagcagaacggctccagcgtgctgagcgaagacaagagcaaaaggctgaataccatattgaacacgatgagcaccatctacagcaccggcaaagtatgcaaccccgacaacccccaagagtgtctgttgttggaacccggcctgaacgagattatggcgaactccctggactacaacgaacgcctgtgggcatgggagtcttggaggtcagaggttggcaagcaactgaggccgttgtacgaagagtacgtggtgctgaagaacgaaatggcacgggccaatcattatgaggactacggtgattattggaggggcgactacgaggtgaacggcgtggacggctacgactatagcaggggccaacttatagaggacgtagagcacacttttgaggaaatcaaacccctgtacgagcacttgcatgcctacgtacgcgccaaactgatgaatgcgtaccccagctacatcagccccatcggctgcctgcccgcacacctgctcggcgacatgtggggccgattctggaccaatctgtatagcctcaccgtgccctttggccagaagccgaacatagatgtgacggatgctatggtagaccaggcgtgggatgcacagcgcatcttcaaggaggccgagaagttcttcgtgagcgtcggcttgcccaacatgacccagggtttctgggagaactcaatgctgactgaccccggcaacgtacagaaagccgtatgccacccaactgcttgggacctgggtaagggagacttccggatcctcatgtgtaccaaggtgaccatggatgactttctcaccgcccacaatgaaatgggcaacatccagtacgatatggcctacgcagcgcagccattccttctgaggaacggtgccaacgagggcttccatgaggcagtcggagagatcatgagcctgtcagcggcgacccctaaacatctgaagagcatcgggctgctgtctccggactttcaagaggacaatgagactgagatcaacttcctcctgaaacaggcgctgaccattgtaggcaccttgcccttcacctacatgctggagaagtggcgatggatggtgtttaagggcgagatcccgaaggaccagtggatgaaaaagtggtgggagatgaagagggagatcgtcggcgtagttgagccggtcccccacgacgaaacctactgcgatcctgccagcctgttccacgtgagcaacgattacagttttatcaggtactacaccaggaccctttaccaatttcagttccaggaggcactgtgccaggccgccaagcacgaaggccccctgcacaagtgcgacatcagcaacagcaccgaggcgggtcaaaagctgtttaacatgctgaggctgggcaagagcgagccgtggaccctggccctggaaaacgttgtgggcgcaaaaaacatgaacgtgaggcccctgctgaactacttcgagcccctgttcacctggctgaaggaccagaacaagaacagtttcgtgggctggagtaccgattggagtcccggtgaatccaagtatggacccccctgtcctccttgccctgctcctgaatttgagggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggcaagtaa

SEQ ID NO: 52 - protein, 13 aa, Artificial Sequence
  source: /note=“Description of Artificial Sequence Synthetic peptide”
GESKYGPPCPPCP

SEQ ID NO: 53 - protein, 9 aa, Artificial Sequence
  source: /note=“Description of Artificial Sequence Synthetic peptide”
GGGGAGGGG

SEQ ID NO: 54 - protein, 14 aa, Artificial Sequence
  source: /note=“Description of Artificial Sequence Synthetic peptide”
GGGGAGGGGAGGGG

SEQ ID NO: 55 - protein, 220 aa, Artificial Sequence
  source: /note=“Description of Artificial Sequence Synthetic polypeptide”
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 834 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGAGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 836 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGAGGGGAGGGGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSP

SEQ ID NO: 58 - DNA, 2562 bp, Artificial Sequence
  source: /note=“Description of Artificial Sequence Synthetic polynucleotide”
atggaatggagctgggtctttctcttcttcctgtcagtaacgactggtgtccactcccagtcaacaatcgaggagcaggcaaagaccttcctggacaagtttaaccacgaagcggaggacctgttctaccagagcagcctggcgagctggaattacaacaccaacatcaccgaggagaacgtgcagaacatgaataacgctggcgacaagtggagcgccttcttgaaggagcaatccaccctggcccagatgtacccgctgcaagagatacagaacctgactgtgaagctgcaactgcaggccctgcagcagaacggctccagcgtgctgagcgaagacaagagcaaaaggctgaataccatattgaacacgatgagcaccatctacagcaccggcaaagtatgcaaccccgacaacccccaagagtgtctgttgttggaacccggcctgaacgagattatggcgaactccctggactacaacgaacgcctgtgggcatgggagtcttggaggtcagaggttggcaagcaactgaggccgttgtacgaagagtacgtggtgctgaagaacgaaatggcacgggccaatcattatgaggactacggtgattattggaggggcgactacgaggtgaacggcgtggacggctacgactatagcaggggccaacttatagaggacgtagagcacacttttgaggaaatcaaacccctgtacgagcacttgcatgcctacgtacgcgccaaactgatgaatgcgtaccccagctacatcagccccatcggctgcctgcccgcacacctgctcggcgacatgtggggccgattctggaccaatctgtatagcctcaccgtgccctttggccagaagccgaacatagatgtgacggatgctatggtagaccaggcgtgggatgcacagcgcatcttcaaggaggccgagaagttcttcgtgagcgtcggcttgcccaacatgacccagggtttctgggagaactcaatgctgactgaccccggcaacgtacagaaagccgtatgccacccaactgcttgggacctgggtaagggagacttccggatcctcatgtgtaccaaggtgaccatggatgactttctcaccgcccacaatgaaatgggcaacatccagtacgatatggcctacgcagcgcagccattccttctgaggaacggtgccaacgagggcttccatgaggcagtcggagagatcatgagcctgtcagcggcgacccctaaacatctgaagagcatcgggctgctgtctccggactttcaagaggacaatgagactgagatcaacttcctcctgaaacaggcgctgaccattgtaggcaccttgcccttcacctacatgctggagaagtggcgatggatggtgtttaagggcgagatcccgaaggaccagtggatgaaaaagtggtgggagatgaagagggagatcgtcggcgtagttgagccggtcccccacgacgaaacctactgcgatcctgccagcctgttccacgtgagcaacgattacagttttatcaggtactacaccaggaccctttaccaatttcagttccaggaggcactgtgccaggccgccaagcacgaaggccccctgcacaagtgcgacatcagcaacagcaccgaggcgggtcaaaagctgtttaacatgctgaggctgggcaagagcgagccgtggaccctggccctggaaaacgttgtgggcgcaaaaaacatgaacgtgaggcccctgctgaactacttcgagcccctgttcacctggctgaaggaccagaacaagaacagtttcgtgggctggagtaccgattggagtccctatgccgacggcggaggtggtgcaggaggcggtggagacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 59 - DNA, 2568 bp, Artificial Sequence
  source: /note=“Description of Artificial Sequence Synthetic polynucleotide”
atggaatggagctgggtctttctcttcttcctgtcagtaacgactggtgtccactccgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggaggtggtgcaggaggcggtggagccggtggcggaggacagtcaacaatcgaggagcaggcaaagaccttcctggacaagtttaaccacgaagcggaggacctgttctaccagagcagcctggcgagctggaattacaacaccaacatcaccgaggagaacgtgcagaacatgaataacgctggcgacaagtggagcgccttcttgaaggagcaatccaccctggcccagatgtacccgctgcaagagatacagaacctgactgtgaagctgcaactgcaggccctgcagcagaacggctccagcgtgctgagcgaagacaagagcaaaaggctgaataccatattgaacacgatgagcaccatctacagcaccggcaaagtatgcaaccccgacaacccccaagagtgtctgttgttggaacccggcctgaacgagattatggcgaactccctggactacaacgaacgcctgtgggcatgggagtcttggaggtcagaggttggcaagcaactgaggccgttgtacgaagagtacgtggtgctgaagaacgaaatggcacgggccaatcattatgaggactacggtgattattggaggggcgactacgaggtgaacggcgtggacggctacgactatagcaggggccaacttatagaggacgtagagcacacttttgaggaaatcaaacccctgtacgagcacttgcatgcctacgtacgcgccaaactgatgaatgcgtaccccagctacatcagccccatcggctgcctgcccgcacacctgctcggcgacatgtggggccgattctggaccaatctgtatagcctcaccgtgccctttggccagaagccgaacatagatgtgacggatgctatggtagaccaggcgtgggatgcacagcgcatcttcaaggaggccgagaagttcttcgtgagcgtcggcttgcccaacatgacccagggtttctgggagaactcaatgctgactgaccccggcaacgtacagaaagccgtatgccacccaactgcttgggacctgggtaagggagacttccggatcctcatgtgtaccaaggtgaccatggatgactttctcaccgcccacaatgaaatgggcaacatccagtacgatatggcctacgcagcgcagccattccttctgaggaacggtgccaacgagggcttccatgaggcagtcggagagatcatgagcctgtcagcggcgacccctaaacatctgaagagcatcgggctgctgtctccggactttcaagaggacaatgagactgagatcaacttcctcctgaaacaggcgctgaccattgtaggcaccttgcccttcacctacatgctggagaagtggcgatggatggtgtttaagggcgagatcccgaaggaccagtggatgaaaaagtggtgggagatgaagagggagatcgtcggcgtagttgagccggtcccccacgacgaaacctactgcgatcctgccagcctgttccacgtgagcaacgattacagttttatcaggtactacaccaggaccctttaccaatttcagttccaggaggcactgtgccaggccgccaagcacgaaggccccctgcacaagtgcgacatcagcaacagcaccgaggcgggtcaaaagctgtttaacatgctgaggctgggcaagagcgagccgtggaccctggccctggaaaacgttgtgggcgcaaaaaacatgaacgtgaggcccctgctgaactacttcgagcccctgttcacctggctgaaggaccagaacaagaacagtttcgtgggctggagtaccgattggagtccctga

SEQ ID NO: 60 - protein, 805 aa, Homo sapiens [sequence omitted]

SEQ ID NO: 61 - protein, 19 aa, Artificial Sequence
  source: /note=“Description of Artificial Sequence Synthetic peptide”
MEWSWVFLFFLSVTTGVHS

SEQ ID NO: 62 - protein, 7 aa, Artificial Sequence
  source: /note=“Description of Artificial Sequence Synthetic peptide”
  MOD_RES (7)..(7): K(Dnp)
YVADAPX